SEQUENCE LISTING (20 sequences)

SEQ ID NO: 1 - DNA, 3255 bp, Homo sapiens
  misc_feature: NELL1 isoform 1
  CDS (154)..(2586)
atatgcgagcgcagcacccggcgctgccgagccacctcccccgccgcccgctagcaagtttggcggctccaagccaggcgcgcctcaggatccaggctcatttgcttccacctagcttcggtgccccctgctaggcggggaccctcgagagcgatgccgatggatttgattttagttgtgtggttctgtgtgtgcactgccaggacagtggtgggctttgggatggaccctgaccttcagatggatatcgtcaccgagcttgaccttgtgaacaccacccttggagttgctcaggtgtctggaatgcacaatgccagcaaagcatttttatttcaagacatagaaagagagatccatgcagctcctcatgtgagtgagaaattaattcagctgttccggaacaagagtgaattcaccattttggccactgtacagcagaagccatccacttcaggagtgatactgtccattcgagaactggagcacagctattttgaactggagagcagtggcctgagggatgagattcggtatcactacatacacaatgggaagccaaggacagaggcacttccttaccgcatggcagatggacaatggcacaaggttgcactgtcagttagcgcctctcatctcctgctccatgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcacacttgcccaacctgcagtgatttcttaagcctggtgcaaggaataatggatttacaagagcttttggccaagatgactgcaaaactaaattatgcagagacaagacttagtcaattggaaaactgtcattgtgagaagacttgtcaagtgagtggactgctctatcgagatcaagactcttgggtagatggtgaccattgcaggaactgcacttgcaaaagtggtgccgtggaatgccgaaggatgtcctgtccccctctcaattgctccccagactccctcccagtgcacattgctggccagtgctgtaaggtctgccgaccaaaatgtatctatggaggaaaagttcttgcagaaggccagcggattttaaccaagagctgtcgggaatgccgaggtggagttttagtaaaaattacagaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgatgagaatgccatctgcaccaacactgtccagggacacagctgcacctgcaaaccgggctacgtggggaacgggaccatctgcagagctttctgtgaagagggctgcagatacggtggaacgtgtgtggctcccaacaaatgtgtctgtccatctggattcacaggaagccactgcgagaaagatattgatgaatgttcagagggaatcattgagtgccacaaccattcccgctgcgttaacctgccagggtggtaccactgtgagtgcagaagcggtttccatgacgatgggacctattcactgtccggggagtcctgtattgacattgatgaatgtgccttaagaactcacacctgttggaacgattctgcctgcatcaacctggcagggggctttgactgtctctgcccctctgggccctcctgctctggtgactgtcctcatgaaggggggctgaagcacaatggccaggtgtggaccttgaaagaagacaggtgttctgtctgctcctgcaaggatggcaagatattctgccgacggacagcttgtgattgccagaatccaagtgctgacctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcacctatgacatcagaaaaacttgcctggacagctatggtgtttcacggcttagtggctcagtgtggacgatggctggatctccctgcacaacctgtaaatgcaagaatggaagagtctgttgttctgtggattttgagtgtcttcaaaataattgaagtatttacagtggactcaacgcagaagaatggacgaaatgaccatccaacgtgattaaggataggaatcggtagtttggtttttttgtttgttttgtttttttaaccacagataattgccaaagtttccacctgaggacggtgtttggaggttgccttttggacctaccactttgctcattcttgctaacctagtctaggtgacctacagtgccgtgcatttaagtcaatggttgttaaaagaagtttcccgtgttgtaaatcatgtttcccttatcagatcatttgcaaatacatttaaatgatctcatggtaaatgttgatgtattttttggtttattttgtgtactaacataatagagagagactcagctccttttatttattttgttgatttatggatcaaattctaaaataaagttgcctgttgtgacttttgtcccatctactgcatacttagtgctgagatccctgtaaaatgttttgatgaaaatatgtatgtagagtccagtcgcattatacatacatttcatagtgctgaaccttcttaaatgcctactcattcagcttaaacaggctgaagccaagtatgacaaagaggggaagggccaaaaacataatcaaagaataattttaaagagaattcttgtctctcttgcaaaaaaaaaa

SEQ ID NO: 2 - protein, 810 aa, Homo sapiens
  misc_feature: NELL1 isoform 1
MPMDLILVVWFCVCTARTVVGFGMDPDLQMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNITYDIRKTCLDSYGVSRLSGSVWTMAGSPCTTCKCKNGRVCCSVDFECLQNN

SEQ ID NO: 3 - DNA, 3114 bp, Homo sapiens
  misc_feature: NELL1 isoform 2
  CDS (154)..(2445)
atatgcgagcgcagcacccggcgctgccgagccacctcccccgccgcccgctagcaagtttggcggctccaagccaggcgcgcctcaggatccaggctcatttgcttccacctagcttcggtgccccctgctaggcggggaccctcgagagcgatgccgatggatttgattttagttgtgtggttctgtgtgtgcactgccaggacagtggtgggctttgggatggaccctgaccttcagatggatatcgtcaccgagcttgaccttgtgaacaccacccttggagttgctcaggtgtctggaatgcacaatgccagcaaagcatttttatttcaagacatagaaagagagatccatgcagctcctcatgtgagtgagaaattaattcagctgttccggaacaagagtgaattcaccattttggccactgtacagcagaagccatccacttcaggagtgatactgtccattcgagaactggagcacagctattttgaactggagagcagtggcctgagggatgagattcggtatcactacatacacaatgggaagccaaggacagaggcacttccttaccgcatggcagatggacaatggcacaaggttgcactgtcagttagcgcctctcatctcctgctccatgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcacacttgcccaacctgcagtgatttcttaagcctggtgcaaggaataatggatttacaagagcttttggccaagatgactgcaaaactaaattatgcagagacaagacttagtcaattggaaaactgtcattgtgagaagacttgtcaagtgagtggactgctctatcgagatcaagactcttgggtagatggtgaccattgcaggaactgcacttgcaaaagtggtgccgtggaatgccgaaggatgtcctgtccccctctcaattgctccccagactccctcccagtgcacattgctggccagtgctgtaaggtctgccgaccaaaatgtatctatggaggaaaagttcttgcagaaggccagcggattttaaccaagagctgtcgggaatgccgaggtggagttttagtaaaaattacagaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgatgagaatgccatctgcaccaacactgtccagggacacagctgcacctgcaaaccgggctacgtggggaacgggaccatctgcagagctttctgtgaagagggctgcagatacggtggaacgtgtgtggctcccaacaaatgtgtctgtccatctggattcacaggaagccactgcgagaaagacattgatgaatgtgccttaagaactcacacctgttggaacgattctgcctgcatcaacctggcagggggctttgactgtctctgcccctctgggccctcctgctctggtgactgtcctcatgaaggggggctgaagcacaatggccaggtgtggaccttgaaagaagacaggtgttctgtctgctcctgcaaggatggcaagatattctgccgacggacagcttgtgattgccagaatccaagtgctgacctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcacctatgacatcagaaaaacttgcctggacagctatggtgtttcacggcttagtggctcagtgtggacgatggctggatctccctgcacaacctgtaaatgcaagaatggaagagtctgttgttctgtggattttgagtgtcttcaaaataattgaagtatttacagtggactcaacgcagaagaatggacgaaatgaccatccaacgtgattaaggataggaatcggtagtttggtttttttgtttgttttgtttttttaaccacagataattgccaaagtttccacctgaggacggtgtttggaggttgccttttggacctaccactttgctcattcttgctaacctagtctaggtgacctacagtgccgtgcatttaagtcaatggttgttaaaagaagtttcccgtgttgtaaatcatgtttcccttatcagatcatttgcaaatacatttaaatgatctcatggtaaatgttgatgtattttttggtttattttgtgtactaacataatagagagagactcagctccttttatttattttgttgatttatggatcaaattctaaaataaagttgcctgttgtgacttttgtcccatctactgcatacttagtgctgagatccctgtaaaatgttttgatgaaaatatgtatgtagagtccagtcgcattatacatacatttcatagtgctgaaccttcttaaatgcctactcattcagcttaaacaggctgaagccaagtatgacaaagaggggaagggccaaaaacataatcaaagaataattttaaagagaattcttgtctctcttgcaaaaaaaaaa

SEQ ID NO: 4 - protein, 763 aa, Homo sapiens
  misc_feature: NELL1 isoform 2
MPMDLILVVWFCVCTARTVVGFGMDPDLQMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNITYDIRKTCLDSYGVSRLSGSVWTMAGSPCTTCKCKNGRVCCSVDFECLQNN

SEQ ID NO: 5 - DNA, 2791 bp, Equus caballus
  misc_feature: NELL1 isoform 1
  CDS (1)..(2376)
atgggctttgggatggaccccgaccttcaaatggatattatcaccgagctcgacctcgtgaacaccacccttggagtcactcaggtgtccggactgcacaatgccagcaaagcatttttatttcaagatgtagagagagagatccatgcagccccacacgtgagtgagaaattaattcagctgttccggaataagagtgaattcacctttttggccactgtgcagcagaagccgtcaacttcaggagtgatactgtccattcgagaactggaaaacagttattttgaactggagagcagtggcctgagagatgagattcgatatcactacacacacaaggggaagcccaggacagaggcacttccctaccggatggcggacggacggtggcacaaggtggcgctgtcagttagcgcctctcatctcctgctccacatcgactgcaacaggatttatgaacgtgtgatagacactcctgagaccaacctccccccaggaagcaatttgtggctgggtcagcgaaaccaaaagcacggcttattcaaaggaatcatccaagatggaaaaatcatcttcatgccgaatggatacataacacagtgtccgaacctgaatcgcacttgcccaacgtgcagtgatttcttaagcctggtgcaaggaatcatggatttacaagagcttctggccaagatgactgcgaaactaaattatgcagagacacgacttagtcaattggaaaactgccactgcgagaagacctgtcaagtgagtggactgctctatagagaccaggactcctgggttgatggcgatcactgtaggaactgcacgtgcaaaagcggcgctgtggaatgtcggaggatgtcttgtccccctctcaattgctccccagactccctccctgtgcacgttgccggccagtgctgtaaggtctgccgaccaaaatgtatctacggagggaaagtccttgcagaaggccagcggattttaaccaagagctgtcgggaatgccgaggtggagttttagtgaaaattacagaagcgtgccctcctttgaactgctcagacaaggatcacattctcccagagaatcagtgctgcagcgtctgcagaggtcataacttttgtgcggaaggacctaaatgtggtgaaaattcagagtgcaaaaactggaatacaaaagctacttgcgagtgcaagaatggttatatctctgtccagggggactccgcctactgtgaagatatcgatgagtgtgctgctaagatgcattactgtcgtgccaatactgtgtgtgtcaacctgcctgggttatatcggtgtgactgtgtcccgggatacattcgcgtggatgatttctcttgtacagaacatgacgaatgtggcagcgggcagcacaactgtgatgagaatgccatctgcaccaacactgtccagggacacagctgcacctgcaaaccgggctacgtggggaatgggaccagctgcagagcgttctgcgaagagggctgcagatatggcgggacatgcgtggctcctaacaaatgtgtctgtccttctggattcacaggaagccactgtgagaaagatattgatgaatgtacagagggaatcattgagtgccacaaccattcccgctgcgttaacctgccagggtggtaccactgtgagtgcagaagcggtttccatgacgatgggacctattcactgtccggggagtcctgtattgacattgatgaatgtgccttaagaactcacacctgttggaatgattctgcctgcatcaacttggcagggggcttcgactgcctgtgtccctcagggccatcctgctctggtgactgcccccacgaaggaggactgaagcgcaacgggcaggtgtggaccctgaaagaagacaggtgttctgtgtgttcctgcaaggatgggaagatattctgccgacggacagcttgtgattgccagaatccaagcgttgaccttttctgttgcccagagtgtgacaccagggtcacaagtcaatgtttagaccaaaatggacacaagctctatcgaagtggagacaattggactcacagctgtcagcagtgccggtgtctggaaggagaggtagattgctggccactcacttgccccagattgagctgtgagtacacagccatcttggaaggggagtgttgtccacgctgtgtcagcgacccctgcctggcggataacatcgtctatgacatcagagaaacttgcctggacagctatggagtttcaaggcttagtggctcagtgtggacattggctggatctccctgcacgacctgcaaatgcaagaatggaagtgtctgctgttctgtggatttggagtgtcttcataataattgaaggatttaaaatggactcatgatcgccagagaaaaatggacaaatgaccatccatgatgatgaaagaacaggagttggtgttttttttaccacagacaattaccaaagtctccgtctgaggaaggtgtttgcaggttgccttttggacctcccactctgctcattcttgctaacctagtctaggtgacctacagtgcatttcagtctatggttgttaaaagaagttttccgtgttgtaaatcacgtttcccttaccaggtcattgcaaatacatttaaatgatttcatggtaaatgttgatgtattttttgggtttattttgtgtactaacataatagagattcagctgcttttatttatttttttcttgacttttggatcaaattcaacaaataaagttgcctgttgtgatttt

SEQ ID NO: 6 - protein, 791 aa, Equus caballus
  misc_feature: NELL1 isoform 1
MGFGMDPDLQMDIITELDLVNTTLGVTQVSGLHNASKAFLFQDVEREIHAAPHVSEKLIQLFRNKSEFTFLATVQQKPSTSGVILSIRELENSYFELESSGLRDEIRYHYTHKGKPRTEALPYRMADGRWHKVALSVSASHLLLHIDCNRIYERVIDTPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHVAGQCCKVCRPKCIYGGKVLAEGQRILTKSCRECRGGVLVKITEACPPLNCSDKDHILPENQCCSVCRGHNFCAEGPKCGENSECKNWNTKATCECKNGYISVQGDSAYCEDIDECAAKMHYCRANTVCVNLPGLYRCDCVPGYIRVDDFSCTEHDECGSGQHNCDENAICTNTVQGHSCTCKPGYVGNGTSCRAFCEEGCRYGGTCVAPNKCVCPSGFTGSHCEKDIDECTEGIIECHNHSRCVNLPGWYHCECRSGFHDDGTYSLSGESCIDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGHKLYRSGDNWTHSCQQCRCLEGEVDCWPLTCPRLSCEYTAILEGECCPRCVSDPCLADNIVYDIRETCLDSYGVSRLSGSVWTLAGSPCTTCKCKNGSVCCSVDLECLHNN

SEQ ID NO: 7 - DNA, 2285 bp, Equus caballus
  misc_feature: NELL1 isoform 2
  CDS (1)..(2235)
atgggctttgggatggaccccgaccttcaaatggatattatcaccgagctcgacctcgtgaacaccacccttggagtcactcaggtgtccggactgcacaatgccagcaaagcatttttatttcaagatgtagagagagagatccatgcagccccacacgtgagtgagaaattaattcagctgttccggaataagagtgaattcacctttttggccactgtgcagcagaagccgtcaacttcaggagtgatactgtccattcgagaactggaaaacagttattttgaactggagagcagtggcctgagagatgagattcgatatcactacacacacaaggggaagcccaggacagaggcacttccctaccggatggcggacggacggtggcacaaggtggcgctgtcagttagcgcctctcatctcctgctccacatcgactgcaacaggatttatgaacgtgtgatagacactcctgagaccaacctccccccaggaagcaatttgtggctgggtcagcgaaaccaaaagcacggcttattcaaaggaatcatccaagatggaaaaatcatcttcatgccgaatggatacataacacagtgtccgaacctgaatcgcacttgcccaacgtgcagtgatttcttaagcctggtgcaaggaatcatggatttacaagagcttctggccaagatgactgcgaaactaaattatgcagagacacgacttagtcaattggaaaactgccactgcgagaagacctgtcaagtgagtggactgctctatagagaccaggactcctgggttgatggcgatcactgtaggaactgcacgtgcaaaagcggcgctgtggaatgtcggaggatgtcttgtccccctctcaattgctccccagactccctccctgtgcacgttgccggccagtgctgtaaggtctgccgaccaaaatgtatctacggagggaaagtccttgcagaaggccagcggattttaaccaagagctgtcgggaatgccgaggtggagttttagtgaaaattacagaagcgtgccctcctttgaactgctcagacaaggatcacattctcccagagaatcagtgctgcagcgtctgcagaggtcataacttttgtgcggaaggacctaaatgtggtgaaaattcagagtgcaaaaactggaatacaaaagctacttgcgagtgcaagaatggttatatctctgtccagggggactccgcctactgtgaagatatcgatgagtgtgctgctaagatgcattactgtcgtgccaatactgtgtgtgtcaacctgcctgggttatatcggtgtgactgtgtcccgggatacattcgcgtggatgatttctcttgtacagaacatgacgaatgtggcagcgggcagcacaactgtgatgagaatgccatctgcaccaacactgtccagggacacagctgcacctgcaaaccgggctacgtggggaatgggaccagctgcagagcgttctgcgaagagggctgcagatatggcgggacatgcgtggctcctaacaaatgtgtctgtccttctggattcacaggaagccactgtgagaaagacattgatgaatgtgccttaagaactcacacctgttggaatgattctgcctgcatcaacttggcagggggcttcgactgcctgtgtccctcagggccatcctgctctggtgactgcccccacgaaggaggactgaagcgcaacgggcaggtgtggaccctgaaagaagacaggtgttctgtgtgttcctgcaaggatgggaagatattctgccgacggacagcttgtgattgccagaatccaagcgttgaccttttctgttgcccagagtgtgacaccagggtcacaagtcaatgtttagaccaaaatggacacaagctctatcgaagtggagacaattggactcacagctgtcagcagtgccggtgtctggaaggagaggtagattgctggccactcacttgccccagattgagctgtgagtacacagccatcttggaaggggagtgttgtccacgctgtgtcagcgacccctgcctggcggataacatcgtctatgacatcagagaaacttgcctggacagctatggagtttcaaggcttagtggctcagtgtggacattggctggatctccctgcacgacctgcaaatgcaagaatggaagtgtctgctgttctgtggatttggagtgtcttcataataattgaaggatttaaaatggactcatgatcgccagagaaaaatggacaaatgacca

SEQ ID NO: 8 - protein, 744 aa, Equus caballus
  misc_feature: NELL1 isoform 2
MGFGMDPDLQMDIITELDLVNTTLGVTQVSGLHNASKAFLFQDVEREIHAAPHVSEKLIQLFRNKSEFTFLATVQQKPSTSGVILSIRELENSYFELESSGLRDEIRYHYTHKGKPRTEALPYRMADGRWHKVALSVSASHLLLHIDCNRIYERVIDTPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHVAGQCCKVCRPKCIYGGKVLAEGQRILTKSCRECRGGVLVKITEACPPLNCSDKDHILPENQCCSVCRGHNFCAEGPKCGENSECKNWNTKATCECKNGYISVQGDSAYCEDIDECAAKMHYCRANTVCVNLPGLYRCDCVPGYIRVDDFSCTEHDECGSGQHNCDENAICTNTVQGHSCTCKPGYVGNGTSCRAFCEEGCRYGGTCVAPNKCVCPSGFTGSHCEKDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGHKLYRSGDNWTHSCQQCRCLEGEVDCWPLTCPRLSCEYTAILEGECCPRCVSDPCLADNIVYDIRETCLDSYGVSRLSGSVWTLAGSPCTTCKCKNGSVCCSVDLECLHNN

SEQ ID NO: 9 - DNA, 2812 bp, Mus musculus
  misc_feature: NELL1
  CDS (40)..(2472)
gcgttggtgcgccctgcttggcggggggcctccggagcgatgccgatggatgtgattttagttttgtggttctgtgtgtgcaccgccaggacagtgctgggctttgggatggaccctgaccttcagatggacatcatcactgaacttgaccttgtgaacaccaccctgggcgtcactcaggtggctggactacacaatgccagtaaggcatttctgtttcaagatgtacagagagagatccactcagcccctcatgtgagtgagaagctgatccagctattccggaataagagtgagtttacctttttggctacagtgcagcagaagccgtccacctcaggggtgatactgtcgatccgggagctggaacacagctattttgaactggagagcagtggcccaagagaagagatacgctatcattacatccatggcggcaagcccaggactgaggcccttccctaccgcatggccgatggacagtggcacaaggtcgcgctgtctgtgagcgcctctcacctcctactccatgtcgactgcaataggatttatgagcgtgtgatagatcctccggagaccaaccttcctccaggaagcaatctatggcttgggcaacgtaatcaaaagcatggctttttcaaaggaatcatccaagatggcaagatcatcttcatgccgaacggcttcatcacacagtgccccaacctaaatcgcacttgcccaacatgcagtgatttcctgagcctggttcaaggaataatggatttgcaagagcttttggccaagatgactgcaaaactgaattatgcagagacgagacttggtcaactggaaaattgccactgtgagaagacctgccaagtgagtgggctgctctacagggaccaagactcctgggtagatggtgacaactgcaggaactgcacatgcaaaagtggtgctgtggagtgccgaaggatgtcctgtcccccactcaactgttccccagactcacttcctgtgcatatttctggccaatgttgtaaagtttgcagaccaaaatgtatctatggaggaaaagttcttgctgagggccagcggattttaaccaagacctgccgggaatgtcgaggtggagtcttggtaaaaatcacagaagcttgccctcctttgaactgctcagagaaggatcatattcttccggagaaccagtgctgcagggtctgccgaggtcataacttctgtgcagaagcacctaagtgtggagaaaactcggaatgcaaaaattggaatacaaaagcgacttgtgagtgcaagaatggatacatctctgtccagggcaactctgcatactgtgaagatatcgatgagtgtgcagcaaagatgcactactgtcatgccaacacggtgtgtgtcaacttgccggggttatatcgctgtgactgcatcccaggatacatccgtgtggatgacttctcttgtacggagcatgatgattgtggcagcggacaacacaactgtgacaaaaatgccatctgtaccaacacagtccagggacacagctgtacctgccagccaggctacgtgggaaatggtactgtctgcaaagcattctgtgaagagggttgcagatacggaggtacctgtgtggcccctaacaaatgtgtctgtccttctggattcacaggaagccactgtgagaaagatattgatgaatgtgcagagggattcgttgagtgccacaaccactcccgctgcgttaaccttccagggtggtaccactgtgagtgcagaagcggtttccatgacgatgggacctattcactgtccggggagtcctgcattgatattgatgaatgtgccttaagaactcacacttgttggaatgactctgcctgcatcaacttagcaggaggatttgactgcctgtgtccctctgggccctcctgctctggtgactgtccccacgaaggggggctgaagcataatgggcaggtgtggattctgagagaagacaggtgttcagtctgttcctgtaaggatgggaagatattctgccggcggacagcttgtgattgccagaatccaaatgttgaccttttctgctgcccagagtgtgacaccagggtcactagccaatgtttagatcaaagcggacagaagctctatcgaagtggagacaactggacccacagctgccagcagtgccgatgtctggaaggagaggcagactgctggcctctagcttgccctagtttgagctgtgaatacacagccatctttgaaggagagtgttgtccccgctgtgtcagtgacccctgcctggctgataatattgcctatgacatcagaaaaacttgcctggacagctctggtatttcgaggctgagcggcgcagtgtggacaatggctggatctccctgtacaacctgtcaatgcaagaatgggagagtctgctgctctgtggatctggtgtgtcttgagaataactgaagattttaaatggactcatcacatgagaaaatggacaaaatgaccatccaacctgaggaagaggaggggctgatttctttttctttttaaccacagtcaattaccaaagtctccatcagaggaaggcgtttgggttgcctttaccactttgctcatccttgctgacctagtctagatgcctgcagtaccgtgtatttcggtcgatggttgttgagtctccgtgctgtaaatcacatttcccttgtcagatcatttacagatacatttaaaggattccatgataaatgttaaagtaccttttgtttattttgtgtaccaacataatagagacttggcacca

SEQ ID NO: 10 - protein, 810 aa, Mus musculus
  misc_feature: NELL1
MPMDVILVLWFCVCTARTVLGFGMDPDLQMDIITELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHVDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCRNCTCKSGAVECRRMSCPPLNCSPDSLPVHISGQCCKVCRPKCIYGGKVLAEGQRILTKTCRECRGGVLVKITEACPPLNCSEKDHILPENQCCRVCRGHNFCAEAPKCGENSECKNWNTKATCECKNGYISVQGNSAYCEDIDECAAKMHYCHANTVCVNLPGLYRCDCIPGYIRVDDFSCTEHDDCGSGQHNCDKNAICTNTVQGHSCTCQPGYVGNGTVCKAFCEEGCRYGGTCVAPNKCVCPSGFTGSHCEKDIDECAEGFVECHNHSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLSCEYTAIFEGECCPRCVSDPCLADNIAYDIRKTCLDSSGISRLSGAVWTMAGSPCTTCQCKNGRVCCSVDLVCLENN

SEQ ID NO: 11 - DNA, 2915 bp, Rattus norvegicus
  misc_feature: NELL1
  CDS (59)..(2491)
aagcactggtttcttgttagcgttggtgcgccctgcttggcgggggttctccggagcgatgccgatggatgtgattttagttttgtggttctgtgtatgcaccgccaggacagtgttgggctttgggatggaccctgaccttcagctggacatcatctcagagctcgacctggtgaacaccaccctgggagtcacgcaggtggctggactgcacaacgccagtaaagcatttctatttcaagatgtacagagagagatccattcggcccctcacgtgagtgagaagctgatccagctattccggaataagagcgagttcacctttttggctacagtgcagcagaaaccatccacctcaggggtgatactgtccatccgggagctggagcacagctattttgaactggagagcagtggcccaagagaagagatacgctaccattacatacatggtggaaagcccaggactgaggcccttccctaccgcatggcagacggacaatggcacaaggtcgcgctgtcagtgagcgcctctcacctcctgctccacatcgactgcaataggatttacgagcgtgtgatagaccctccggagaccaaccttcctccaggaagcaatctgtggcttgggcaacgtaaccaaaagcatggctttttcaaaggaatcatccaagatggtaagatcatcttcatgccgaatggtttcatcacacagtgtcccaacctcaatcgcacttgcccaacatgcagtgacttcctgagcctggttcaaggaataatggatttgcaagagcttttggccaagatgactgcaaaactgaattatgcagagacgagacttggtcaactggaaaattgccactgtgagaagacctgccaagtgagtgggctgctctacagggaccaagactcctgggtggatggtgacaactgtgggaactgcacgtgcaaaagtggtgccgtggagtgccgcaggatgtcctgtcccccgctcaactgttccccggactcacttcctgtgcacatttccggccagtgttgtaaagtttgcagaccaaaatgtatctatggaggaaaagttcttgctgagggccagcggattttaaccaagacctgccgggaatgtcgaggtggagtcttggtaaaaatcacagaagcttgccctcctttgaactgctcagcaaaggatcatattcttccagagaatcagtgctgcagggtctgcccaggtcataacttctgtgcagaagcacctaagtgcggagaaaactcggaatgcaaaaattggaatacaaaagcaacctgtgagtgcaagaatggatacatctctgtccagggcaactctgcatactgtgaagatattgatgagtgtgcagctaaaatgcactattgtcatgccaacaccgtgtgtgtcaacttgccggggttgtatcgctgtgactgcgtcccagggtacatccgtgtggatgacttctcttgtacggagcatgatgattgtggcagcggacaacacaactgcgacaaaaatgccatctgtaccaacacagtccagggacacagctgcacctgccagccgggttacgtgggaaatggcaccatctgcaaagcattctgtgaagagggttgcagatacggaggtacctgtgtggctcctaacaagtgtgtctgtccttctggattcacgggaagccactgtgagaaagatattgatgaatgcgcagagggattcgttgaatgccacaactactcccgctgtgttaacctgccagggtggtaccactgtgagtgcagaagcggtttccatgacgatgggacctactcactgtccggggagtcctgcattgatatcgatgaatgtgccttaagaactcacacttgttggaatgactctgcctgcatcaacttagcaggaggatttgactgcctgtgtccctctgggccctcctgctctggtgactgtccccacgaaggagggctgaagcataatgggcaggtgtggattctgagagaagacaggtgttcagtctgttcctgcaaggatgggaagatattctgccggcggacagcttgtgattgccagaatccaaatgttgaccttttttgctgcccagagtgcgataccagggtcaccagccaatgtttagatcaaagtggacagaagctctatcgaagtggagacaactggacccacagctgccagcagtgccgatgtctggaaggagaggcagactgctggcctctggcttgccctagtttgggctgtgaatacacagccatgtttgaaggggagtgttgtccccgatgtgtcagtgacccctgcctggctggtaatattgcctatgacatcagaaaaacttgcctggacagctttggtgtttcgaggctgagcggagccgtgtggacaatggctggatctccttgtacaacctgcaaatgcaagaatgggagagtctgctgctctgtggatctggagtgtattgagaataactgaagattttaaatggactcgtcacgtgagaaaatgggcaaaatgatcatcccacctgaggaagaagaggggctgatttctttttctttttaaccacagtcaattaccaaagtctccatctgaggaaggcgtttggattgcctttgccactttgctcatccttgctgacctagtctagatgcctgcagtaccgtgcatttcggtcgatggttgttgagtctcagtgttgtaaatcgcatttccctcgtcagatcatttacagatacatttaaaggggttccatgataaatgttaatgtaacttttgtttattttgtgtactgacataatagagacttggcaccatttatttatttttcttgatttttggatcaaattctaaaaataaagttgcctgttgcgaaaaaaaaaaaaaaaaaaaaaaaaaa

SEQ ID NO: 12 - protein, 810 aa, Rattus norvegicus
  misc_feature: NELL1
MPMDVILVLWFCVCTARTVLGFGMDPDLQLDIISELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHIDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCGNCTCKSGAVECRRMSCPPLNCSPDSLPVHISGQCCKVCRPKCIYGGKVLAEGQRILTKTCRECRGGVLVKITEACPPLNCSAKDHILPENQCCRVCPGHNFCAEAPKCGENSECKNWNTKATCECKNGYISVQGNSAYCEDIDECAAKMHYCHANTVCVNLPGLYRCDCVPGYIRVDDFSCTEHDDCGSGQHNCDKNAICTNTVQGHSCTCQPGYVGNGTICKAFCEEGCRYGGTCVAPNKCVCPSGFTGSHCEKDIDECAEGFVECHNYSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLGCEYTAMFEGECCPRCVSDPCLAGNIAYDIRKTCLDSFGVSRLSGAVWTMAGSPCTTCKCKNGRVCCSVDLECIENN

SEQ ID NO: 13 - protein, 810 aa, Felis catus
  MISC_FEATURE: NELL1 isoform 1
MPRDVILVVWFCVCTARTVVGFGTDPDLQVDIIAELDLVNTTAGVTQVSGLHNASKAYLFQETEREIHAAPHVSEKLIQLFRNKSEFSFLATVQQKPSTSGVILSIRELEHSYFELESSGLRDEIRYHYIHNGKPRTEALPYRMADGQWHKVALSISASHLLLHVDCNRIYERVIDPPETNLPPGSNVWLGQRNQKHGLFKGIIQDGKIIFMPNGYITQCPNLNRTCPTCSDFLSLVQGIMDLQELLAKMTAKLNYAETRLNQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITDACPPLNCSEKDHILPENQCCSVCRGHNFCAEGPTCGENSECKNWNTKATCECKNGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCQEGCRYGGTCVSPNKCVCPSGFTGSHCEKDIDECTEGIIECHNHSRCVNLPGWYHCECRSGFHDDGTYSLSGESCIDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGHKLYRSGDNWTHSCQQCRCLEGEVDCWPLTCPNLSCEYTAMLEGECCPRCVSDPCLADNIAYDIRKTCLDSYGISRLSGAVWTMAGSPCTTCKCKNGSVCCSVDLECLHNN

SEQ ID NO: 14 - protein, 763 aa, Felis catus
  MISC_FEATURE: NELL1 isoform 2
MPRDVILVVWFCVCTARTVVGFGTDPDLQVDIIAELDLVNTTAGVTQVSGLHNASKAYLFQETEREIHAAPHVSEKLIQLFRNKSEFSFLATVQQKPSTSGVILSIRELEHSYFELESSGLRDEIRYHYIHNGKPRTEALPYRMADGQWHKVALSISASHLLLHVDCNRIYERVIDPPETNLPPGSNVWLGQRNQKHGLFKGIIQDGKIIFMPNGYITQCPNLNRTCPTCSDFLSLVQGIMDLQELLAKMTAKLNYAETRLNQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITDACPPLNCSEKDHILPENQCCSVCRGHNFCAEGPTCGENSECKNWNTKATCECKNGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCQEGCRYGGTCVSPNKCVCPSGFTGSHCEKDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGHKLYRSGDNWTHSCQQCRCLEGEVDCWPLTCPNLSCEYTAMLEGECCPRCVSDPCLADNIAYDIRKTCLDSYGISRLSGAVWTMAGSPCTTCKCKNGSVCCSVDLECLHNN

SEQ ID NO: 15 - protein, 901 aa, Canis lupis familiaris
  MISC_FEATURE: NELL1
MTSTSFLLWLGCVHNTKFPFPLVLVTRAIVVVVVEVVGVGSPGVRIRSTGCDILLLYEVLEHLLGIRFLCVDQGENSCHHGQCACRLQVIVPKALMSVFEAKTAVCFFPVVGFGTDPDLQMDIITELDLVNISLGVTQVSGLHNASKAYVFQDTAREIHAAPHVSEKLIQLFRNKSDFTFLATVQQKPSTSGVILSIRELEHSYFELESSGLRDEIRYHYMHNGKPRTEALPYRLADGQWHKVALSVSASHLLLHIDCNRIYERVIDPPETNLPPGSNLWLGQRNQKHGFFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKGGAVECRRMSCPPLNCSPDSLPVHIAGQCCKVCRPKCIYGGRVLAEGQRILTKSCRECRGGVLVKITDACPPLNCSEKDHILPENQCCSVCRGHNFCAEGPKCGENSECKNWNTKATCECKNGYISVQGDSAYCEDIDECAAKMHYCHANTVCVNLPGLYRCDCVPGYIRVDDFSCTEHDECGSGQHNCDENAICTNTVRGHSCTCKPGYVGNGTICRAFCQEGCRYGGSCVSPNKCVCPSGFTGSHCEKDIDECTEGIIECHNHSRCVNLPGWYHCECRSGFHDDGTYSLSGESCIDIDECALRTHTCWNDSACINLAGGFDCLCPSGPSCSGDCPHEGGLKRNGQVWTLKEDRCSVCSCKDGKILCRRTACDCQNPSVDLFCCPECDTRVTSQCLDQNGHKLYRSGDNWTHSCQQCRCLEGEVDCWPLTCPNLSCEYTAILEGECCPRCVSDPCLADNIAYDIRKTCLDSYGISRLSGSVWTMAGSPCTTCKCKNGSVCCSVDLECLHNN

SEQ ID NO: 16 - protein, 810 aa, Ovis aries
  MISC_FEATURE: NELL1
MPRGVILVVCFCVCAARTVVGFGMDPDLQLDIITELDLVNTTLGVTQVSGLHNTSKAFLFQDAEREIHAAPHVSEKLIQLFRNKSEFTFLATVQQKPSTSGVILSIRELEHSYFELESSGLRDEIRYHYMHSGRPRTEALPYRLADGQWHRVALSVSASHLLLHIDCNRIYERVIDPPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILSKNCQECRGGVLVKITEACPLLNCSEKDHILPENQCCSVCRGHNFCAEGPKCGENSECKNWNTKATCECKNGYISVQGDSAYCEDIDECAAKMHYCHANTVCVNLPGLYRCDCVPGYIRVDDFSCTEHDDCGSGQHNCDENAICTNTVQGHSCTCKPGYVGNGTICRAFCEEGCRYGGTCMAPNKCVCPSGFTGSHCEKDIDECAEGIIECHSHSRCVNLPGWYHCECRSGFHDDGTYSLSGESCVDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGNKLYRSGDNWTHSCQQCRCLEGEVDCWPLTCPSLSCEYTTILEGECCPRCVSDPCLADNIAYDIRKTCLDSYGLSRLSGSVWTMAGSPCTTCKCKNGSVCCSVDLECLHNN

SEQ ID NO: 17 - protein, 610 aa, Homo sapiens
  MISC_FEATURE: NELL1 fragment
FGMDPDLQMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCS

SEQ ID NO: 18 - protein, 610 aa, Equus caballus
  MISC_FEATURE: NELL1 fragment
FGMDPDLQMDIITELDLVNTTLGVTQVSGLHNASKAFLFQDVEREIHAAPHVSEKLIQLFRNKSEFTFLATVQQKPSTSGVILSIRELENSYFELESSGLRDEIRYHYTHKGKPRTEALPYRMADGRWHKVALSVSASHLLLHIDCNRIYERVIDTPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHVAGQCCKVCRPKCIYGGKVLAEGQRILTKSCRECRGGVLVKITEACPPLNCSDKDHILPENQCCSVCRGHNFCAEGPKCGENSECKNWNTKATCECKNGYISVQGDSAYCEDIDECAAKMHYCRANTVCVNLPGLYRCDCVPGYIRVDDFSCTEHDECGSGQHNCDENAICTNTVQGHSCTCKPGYVGNGTSCRAFCEEGCRYGGTCVAPNKCVCPSGFTGSHCEKDIDECTEGIIECHNHSRCVNLPGWYHCECRSGFHDDGTYSLSGESCIDIDECALRTHTCWNDSACINLAGGFDCLCPSGPSCS

SEQ ID NO: 19 - protein, 599 aa, Bos taurus
  MISC_FEATURE: NELL1
MALCSFSVVGFGLDPDLQLDIITELDLVNTTLGVTQVSGLHNTSKAFLFQDAEREIHAAPHVSEKLIQLFRNKSEFTFLATVQQKPSTSGVILSIRELEHSYFELESSGLRDEIRYHYVHSGRPRTEALPYRLADGQWHRVALSVSASHLLLHIDCNRIYERVIDPPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGECCKVCRPKCIYGGKVLAEGQRILSKSCQECRGGVLVKITEACPLLNCSEKDHILPENQCCSVCRGHNFCAEGPKCGENSECKNWNTKATCECKNGYISVQGDSAYCEDIDECAAKMHYCHANTVCVNLPGLYRCDCVPGYIRVDDFSCTEHDDCGSGQHNCDENAICTNTVQGHSCTCKPGYVGNGTICRGMPEVGPPRALLNSLDLGFLSFSKEALAVGMITLEGNIVAKSFTDDETLVERGREKVIALLFSWLHKEKLSLENLRDIYCKANSLVGLDHLPQR

SEQ ID NO: 20 - protein, 269 aa, Homo sapiens
  MISC_FEATURE: interleukin-1 beta proprotein
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

(19) United States
(12) Patent Application Publication
(10) Pub. No.: US 2023/0121387 A1
(43) Pub. Date: Apr. 20, 2023

(54) METHODS AND COMPOSITIONS FOR TREATING MUSCLE ATROPHY

(71) Applicant: NELLONE THERAPEUTICS, INC., OAK RIDGE, TN (US)

(72) Inventors: CYMBELINE T. CULIAT, OAK RIDGE, TN (US); SHANNON STEWART EAKER, KNOXVILLE, TN (US)

(73) Assignee: NELLONE THERAPEUTICS, INC., OAK RIDGE, TN (US)

(21) Appl. No.: 17/908,075

(22) PCT Filed: Mar. 5, 2021
(86) PCT No.: PCT/US2021/021191

§ 371 Date: Aug. 30, 2022

Related U.S. Application Data
(60) Provisional application No. 62/986,327, filed on Mar. 6, 2020.

(51) Int. Cl. A61K 38/18; A61P 21/00

(52) U.S. Cl. CPC A61K 38/18; A61P 21/00

(57) ABSTRACT

Provided are methods of treating muscle atrophy by administering a NELL1 polypeptide, or a nucleic acid molecule encoding a NELL1 polypeptide, to a subject in need thereof. Such subjects include pediatric subjects, cancer patients, patients with relatively high circulating levels of interleukin-1 beta (IL-1β), and patients suffering from chronic systemic inflammation, particularly inflammation associated with interleukin-1 beta (IL-1β).

### CROSS REFERENCED APPLICATIONS

This application claims the benefit of U.S. Provisional Application No. 62/986,327, filed Mar. 6, 2020, which is incorporated herein by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named N88509_1070WO_SL_ST25.txt, created on Mar. 5, 2021, and having a size of 161,872 bytes. The sequence listing contained in this ASCII formatted document is part of the specification and is herein incorporated by reference in its entirety.

### FIELD OF THE INVENTION

This application generally relates to the healing of muscle atrophy with a NELL1 protein or a nucleic acid encoding the same.

### BACKGROUND OF THE INVENTION

Muscle wasting or atrophy (MA) is a debilitating condition affecting at least 35 million Americans, a leading cause of death in cancer and cardiac patients, and incurs billions of dollars of annual healthcare costs. MA is caused by many factors, but different types follow a common pathway reflected by a similar program of gene expression in biological processes such as: inflammation, energy production and consumption, protein degradation and synthesis, and muscle growth and differentiation (Lecker S H et al. 2017 The FASEB Journal 18(1):39-51). Muscle wasting syndrome has a marked detrimental effect on the quality of life and survival of patients afflicted with cancer, chronic obstructive pulmonary disease (COPD), chronic heart failure, AIDS, chronic kidney disease and other conditions (Seelander M et al. 2015 Mediators of Inflammation Vol 2015, Article ID 536954). Disruptions in muscle metabolic processes drive the rapid loss of muscle in MA and are believed to be the molecular basis of cancer-induced MA or cachexia found in 80% of cancers and the direct cause for at least 20% of cancer deaths (Porporato P E 2016 Oncogenesis 5:e200, doi:10.1038/oncsis.2016.3). These metabolic disruptions are typically initiated and aggravated by chronic systemic inflammation manifested in the majority of MA patients (Seelander M et al. 2015; Argiles J M et al. 2006 Cachexia and Wasting: A Modern Approach Springer-Verlag, Chapter 9.2, pp. 467-475).

Despite many efforts (at least 19 drug candidates) to develop a therapeutic for this life-threatening condition, an effective solution has remained elusive—and the failures of clinical trials for initially promising candidates have been discouraging (Dutts V et al. 2015 Pharmacological Research 99:86-100; Morley J E et al. 2014 J Cachexia Sarcopenia Muscle 5:83-87; Lok C 2015 Nature 528:182-183; Mueller T C et al. 2016 BMC Cancer 16:75, doi:10.1186/s12885-016-2121-8). Many of these ineffective drug candidates were well characterized genes or proteins involved in muscle biology (e.g., proteins or factors that promote muscle formation or inhibit protein degradation), underscoring that developing a therapeutic for treating MA is neither obvious nor straightforward.

### SUMMARY OF THE INVENTION

Methods for treating muscle atrophy are provided. The methods comprise administering to a subject in need thereof an effective amount of a NELL1 polypeptide or a nucleic acid encoding the same. Specific methods that are provided include methods of treating muscle atrophy in a pediatric subject in need thereof by administering an effective amount of a NELL1 polypeptide of a nucleic acid encoding the same. In some of these embodiments, the pediatric subject has chronic systemic inflammation. In certain embodiments, the chronic systemic inflammation is due to a viral infection. In some of these embodiments, the viral infection is by a coronavirus. In certain embodiments, the coronavirus is SARS-CoV-2. The pediatric subject can have increased circulating levels of IL-1β when compared to a control subject. In some of these embodiments, the pediatric subject has a genetic predisposition for increased circulating levels of IL-1β when compared to a control subject. In certain embodiments, the pediatric subject has increased circulating levels of interleukin-8 (IL-8), nuclear factor kappa-light chain-enhancer of activated B cells (NF-κB), and matrix metalloproteinase 1 (MMP1) when compared to a control subject. The muscle atrophy treated with NELL1 can be cardiac muscle atrophy or skeletal muscle atrophy. In certain embodiments, the NELL1 polypeptide or nucleic acid molecule encoding the same is administered locally to the atrophied muscle. In other embodiments, the NELL1 polypeptide or nucleic acid molecule encoding the same is administered systemically and can be administered via an intravenous, subcutaneous, intramuscular, intra-arterial, or intraperitoneal route. In still other embodiments, the NELL1 polypeptide or nucleic acid molecule encoding the same is incorporated into a drug eluting device, scaffold, matrix, or sutures. The pediatric subject administered a NELL1 peptide or nucleic acid encoding the same can be a mammal, such as a human, cat, dog, or horse. In certain embodiments, the NELL1 polypeptide has an amino acid sequence having at least 80% sequence identity to the amino acid sequence set forth as SEQ ID NO: 2, 4, 6, 10, or 12. In some of these embodiments, the NELL1 polypeptide is the polypeptide of SEQ ID NO: 2, 4, 6, 10, 12, 17, or 18. In some of those embodiments wherein the method comprises administering a nucleic acid molecule encoding a NELL1 polypeptide, the nucleic acid molecule is comprised within an expression vector and operably linked to a promoter.

In another aspect, methods for treating muscle atrophy in a subject with chronic systemic inflammation are provided, wherein the method comprises administering an effective amount of a NELL1 polypeptide, or a nucleic acid molecule encoding the same. In some embodiments, the subject is a pediatric subject. In certain embodiments, the subject has a cancer, which can be a stage III or IV cancer. The cancer can also be a pancreatic or gastric cancer. In some embodiments, the subject has increased circulating levels of IL-1β when compared to a control subject. In some of these embodiments, the subject has a genetic predisposition for increased circulating levels of IL-1β when compared to a control subject. In certain embodiments, the subject has increased circulating levels of IL-8, NF-κB, and MMP1 when compared to a control subject. In some embodiments, the chronic systemic inflammation is due to a viral infection. In some of these embodiments, the viral infection is by a coronavirus. In some embodiments, the coronavirus is SARS-CoV-2. The muscle atrophy treated with NELL1 can be cardiac muscle atrophy or skeletal muscle atrophy. In certain embodiments, the NELL1 polypeptide or nucleic acid molecule encoding the same is administered locally to the atrophied muscle. In other embodiments, the NELL1 polypeptide or nucleic acid molecule encoding the same is administered systemically and can be administered via an intravenous, subcutaneous, intramuscular, intra-arterial, or intraperitoneal route. In still other embodiments, the NELL1 polypeptide or nucleic acid molecule encoding the same is incorporated into a drug eluting device, scaffold, matrix, or sutures. The subject administered a NELL1 peptide or nucleic acid encoding the same can be a mammal, such as a human, cat, dog, or horse. In certain embodiments, the NELL1 polypeptide has an amino acid sequence having at least 80% sequence identity to the amino acid sequence set forth as SEQ ID NO: 2, 4, 6, 10, or 12. In some of these embodiments, the NELL1 polypeptide is the polypeptide of SEQ ID NO: 2, 4, 6, 10, 12, 17, or 18. In some of those embodiments wherein the method comprises administering a nucleic acid molecule encoding a NELL1 polypeptide, the nucleic acid molecule is comprised within an expression vector and operably linked to a promoter.

In yet another aspect, methods for treating muscle atrophy in a subject with increased circulating levels of IL-1β when compared to a control subject are provided, wherein the method comprises administering an effective amount of a NELL1 polypeptide or a nucleic acid molecule encoding the same. In some embodiments, the subject is a pediatric subject. In certain embodiments, the subject has a cancer, which can be a stage III or IV cancer. The cancer can also be a pancreatic or gastric cancer. In some embodiments, the subject has chronic systemic inflammation. In some embodiments, the chronic systemic inflammation is due to a viral infection. In some of these embodiments, the viral infection is by a coronavirus. In some embodiments, the coronavirus is SARS-CoV-2. In certain embodiments, the subject has a genetic predisposition for increased circulating levels of IL-1B when compared to a control subject. In some embodiments, the subject has increased circulating levels of IL-8, NF-κB, and MMP1 when compared to a control subject. The muscle atrophy treated with NELL1 can be cardiac muscle atrophy or skeletal muscle atrophy. In certain embodiments, the NELL1 polypeptide or nucleic acid molecule encoding the same is administered locally to the atrophied muscle. In other embodiments, the NELL1 polypeptide or nucleic acid molecule encoding the same is administered systemically and can be administered via an intravenous, subcutaneous, intramuscular, intra-arterial, or intraperitoneal route. In still other embodiments, the NELL1 polypeptide or nucleic acid molecule encoding the same is incorporated into a drug eluting device, scaffold, matrix, or sutures. The subject administered a NELL1 peptide or nucleic acid encoding the same can be a mammal, such as a human, cat, dog, or horse. In certain embodiments, the NELL1 polypeptide has an amino acid sequence having at least 80% sequence identity to the amino acid sequence set forth as SEQ ID NO: 2, 4, 6, 10, or 12. In some of these embodiments, the NELL1 polypeptide is the polypeptide of SEQ ID NO: 2, 4, 6, 10, 12, 17, or 18. In some of those embodiments wherein the method comprises administering a nucleic acid molecule encoding a NELL1 polypeptide, the nucleic acid molecule is comprised within an expression vector and operably linked to a promoter.

In still another aspect, methods for treating muscle atrophy in a subject with a cancer are provided, wherein the method comprises administering an effective amount of a NELL1 polypeptide, or a nucleic acid molecule encoding the same. The cancer can be a stage III or IV cancer. In some embodiments, the cancer is a pancreatic or gastric cancer. In some embodiments, the subject has increased circulating levels of IL-1β when compared to a control subject. In some of these embodiments, the subject has a genetic predisposition for increased circulating levels of IL-1β when compared to a control subject. In certain embodiments, the subject has increased circulating levels of IL-8, NF-κB, and MMP1 when compared to a control subject. The muscle atrophy treated with NELL1 can be cardiac muscle atrophy or skeletal muscle atrophy. The subject administered a NELL1 peptide or nucleic acid encoding the same can be a mammal, such as a human, cat, dog, or horse. In certain embodiments, the NELL1 polypeptide has an amino acid sequence having at least 80% sequence identity to the amino acid sequence set forth as SEQ ID NO: 2, 4, 6, 10, or 12. In some of these embodiments, the NELL1 polypeptide is the polypeptide of SEQ ID NO: 2, 4, 6, 10, 12, 17, or 18. In some of those embodiments wherein the method comprises administering a nucleic acid molecule encoding a NELL1 polypeptide, the nucleic acid molecule is comprised within an expression vector and operably linked to a promoter.

In another aspect, methods for treating muscle atrophy or cachexia in a subject having systemic inflammation due to a viral infection are provided. In certain embodiments, the viral infection is by a coronavirus. In some of these embodiments, the coronavirus is SARS-CoV-2. The muscle atrophy treated with NELL1 can be cardiac muscle atrophy or skeletal muscle atrophy. The subject administered a NELL1 peptide or nucleic acid encoding the same can be a mammal, such as a human, cat, dog, or horse. In certain embodiments, the NELL1 polypeptide has an amino acid sequence having at least 80% sequence identity to the amino acid sequence set forth as SEQ ID NO: 2, 4, 6, 10, or 12. In some of these embodiments, the NELL1 polypeptide is the polypeptide of SEQ ID NO: 2, 4, 6, 10, 12, 17, or 18. In some of those embodiments wherein the method comprises administering a nucleic acid molecule encoding a NELL1 polypeptide, the nucleic acid molecule is comprised within an expression vector and operably linked to a promoter. In another aspect, methods for treating muscle atrophy in a subject in need thereof are provided, wherein the method comprises administering an effective amount of a NELL1 polypeptide, or a nucleic acid molecule encoding the same, wherein said NELL1 polypeptide has an amino acid sequence having at least 80% sequence identity to the amino acid sequence set forth as SEQ ID NO: 17 or 18. In some of these embodiments, the NELL1 polypeptide is the polypeptide of SEQ ID NO: 17 or 18. In some embodiments, the NELL1 polypeptide has one of the following properties: enhanced efficacy in tissue regeneration, enhanced prevention of tissue loss, promotion of wound healing, easier purification, higher yield, and less aggregate formation, when compared to the polypeptide's respective full-length NELL1 protein. In some of these embodiments, the NELL1 polypeptide lacks the carboxy-terminal 179 amino acid residues of the NELL1 polypeptide's respective full-length NELL1 protein. In certain embodiments, the subject is a pediatric subject. In some embodiments, the subject has chronic systemic inflammation. In some embodiments, the chronic systemic inflammation is due to a viral infection. In some of these embodiments, the viral infection is by a coronavirus. In some embodiments, the coronavirus is SARS-CoV-2. In certain embodiments, the subject has increased circulating levels of IL-1β when compared to a control subject. In some of these embodiments, the subject has a genetic predisposition for increased circulating levels of IL-1β when compared to a control subject. In certain embodiments, the subject has increased circulating levels of IL-8, NF-κB, and MMP1 when compared to a control subject. In some embodiments, the subject has a cancer, which can be a stage III or IV cancer. In some of these embodiments, the cancer is a pancreatic or gastric cancer. The muscle atrophy can be skeletal muscle atrophy and/or cardiac muscle atrophy. In certain embodiments, the NELL1 polypeptide or nucleic acid molecule encoding the same is administered locally to the atrophied muscle. In other embodiments, the NELL1 polypeptide or nucleic acid molecule encoding the same is administered systemically and can be administered via an intravenous, subcutaneous, intramuscular, intra-arterial, or intraperitoneal route. In still other embodiments, the NELL1 polypeptide or nucleic acid molecule encoding the same is incorporated into a drug eluting device, scaffold, matrix, or sutures. The subject administered a NELL1 peptide or nucleic acid encoding the same can be a mammal, such as a human, cat, dog, or horse. In some of those embodiments wherein the method comprises administering a nucleic acid molecule encoding a NELL1 polypeptide, the nucleic acid molecule is comprised within an expression vector and operably linked to a promoter.

The foregoing is a summary and thus contains, by necessity, simplifications, generalizations, and omissions of detail; consequently, those skilled in the art will appreciate that the summary is illustrative and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE FIGURES

FIG. 1 demonstrates functional myotube atrophy rescue using CYTOO's MYOSCREEN™ assay. Pediatric (AA179) myotubes were treated with (100 ng/ml) insulin-like growth factor-1 (IGF-1) and/or 20 ng/ml interleukin-1-beta (IL-1β) for four days, after which the cells were prepared for immunostaining and the nuclei counted (FIG. 1A), the mean area of the myotubes measured (FIG. 1C), and the fusion index calculated (FIG. 1B). Control and IGF1 treatment alone samples were performed in triplicate and the IL-1β, and IL-1β and IGF-1 treatments were performed in quadruplicate. The IL-1β/IGF-1 treatments were compared to IL-1β alone with a T-test. *p<0.05; **p<0.01; ***p<0.001; ****p<0.0001.

FIG. 2 depicts the rescue of IL-1β-induced muscle atrophy by the NELL1 NV1 fragment in the atrophy rescue assay. Pediatric (AA179) myotubes were treated with 20 ng/ml IL-1β, and five concentrations of the NELL1 NV1 fragment for four days, after which the cells were prepared for immunostaining and the nuclei counted (FIG. 2A), the mean area of the myotubes measured (FIG. 2C), and the fusion index calculated (FIG. 2B). All treatments were performed in quadruplicate, with the exception of the 1 μg/ml concentration of NV1 which was tested in triplicate. A one-way ANOVA test was used to compare the IL-1β condition vs. IL-1β and NV1 conditions. *p<0.05; **p<0.01; ***p<0.001.

### DETAILED DESCRIPTION OF THE INVENTION

### I. Introduction

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes and are not to be construed as limiting the subject matter described. Finally, for the purposes of the instant disclosure all identifying sequence Accession numbers may be found in the NCBI Reference Sequence (RefSeq) database and/or the NCBI GenBank archival sequence database unless otherwise noted.

Described herein is a direct test of the capabilities of a fragment of the NELL1 signaling protein (set forth as SEQ ID NO: 17 and described in U.S. Patent Application Publication No. 2018/0057550, which is herein incorporated by reference in its entirety, and specifically the NELL1 proteins and variants described therein) to rescue muscle atrophy in a human in vitro model (Young et al. 2018 SLAS Discov 23(8):790-806, doi:10.1177/2472555218761102). MA was induced by the major pro-inflammatory cytokine IL-1β and 5 concentrations of NV1 were tested (0.2, 0.4, 0.6, 0.8 and 1 μg/ml). Full rescue of the pediatric muscle tissue (statistically significant recovery of the fusion index and myotube size) in IL-1β-induced MA was observed at several concentrations tested (0.2, 0.4 and 1 μg/ml of NV1), manifesting a surprising and unexpected U-Shaped dose curve. These data support the application of NV1 and other NELL1 polypeptides in treating MA in patients who suffer with or are highly sensitive to chronic inflammation mediated by IL-1β.

The dramatic rescue of the IL-1β-induced MA phenotype in muscle tissue by a NELL1 polypeptide as described herein also supports using NELL1 to treat cancer cachexia. IL-1β is a cytokine that is highly elevated during chronic inflammation and when this occurs within a cancer environment, it promotes muscle wasting (i.e., cancer cachexia), tumor development and metastases. (Argiles J M et al. 2006; Graziano F et al. 2005 J Clin Oncol 23(10):2339-45; Melstrom L G et al. 2007 Histol Histopath 22(7):805-14; Bent et al. 2018 Int. J. Mol. Sci. 19, 2155, doi:10.3390/ijms19082155). The impact of IL-1β pro-inflammatory pathways in muscle wasting is particularly pronounced in advanced pancreatic and gastric cancer, the two cancers most prone to cachexia (Melstrom L G et al. 2007; Graziano F et al. 2005; Zhang D et al. 2007 BMC Cancer 7:45). Cancer cachexia results in severe muscle wasting in both skeletal and cardiac muscles in lung, pancreatic and GI cancers (Barkhudaryan A et al. 2017 ESC Heart Failure 4:458-567). The data disclosed herein demonstrating that NELL1 can rescue IL-1β-induced MA and the involvement of IL-1β in muscle wasting associated with cancer cachexia supports treating these patients with NELL1 therapy.

### II. NELL1

The neural epidermal growth-factor-like (nel) gene was first detected in neural tissue from an embryonic chicken cDNA library, and its human ortholog neural epidermal growth-factor-like 1 (NEL-like 1, NELL1) was discovered later in B-cells. Studies have reported the presence of NELL1 in various fetal and adult organs, including, but not limited to, skeletal and cardiac muscle, skin, the brain, kidneys, colon, thymus, lung, and small intestine.

The human NELL1 gene encodes an 810-amino acid polypeptide. Generally, the arrangement of the functional domains of the NELL1 protein bears resemblance to thrombospondin-1 (THBS1) and consists of a thrombospondin N-terminal domain (TSPN) and several von Willebrand factor, type C (VWC), and epidermal growth-factor (EGF) domains. A domain is a region of a protein with a characteristic primary structure and function.

Additional studies have shown that there are at least two human NELL1 transcript variants encoding different isoforms. In humans, the nel-like 1 isoform 1 precursor transcript variant (set forth in SEQ ID NO: 1) represents the longer transcript (set forth in GenBank Acc. No. NM_006157) and encodes the longer isoform 1 (set forth in SEQ ID NO: 2).

The conserved domains of NELL1 reside in seven regions of the isoform 1 peptide and include: (1) a TSPN domain/Laminin G superfamily domain; (2) a VWC domain; (3) four EGF-like domains; and (4) a VWC domain. NELL1 also comprises a secretion signal peptide domain (amino acid residues 1-16 of SEQ ID NO: 2) that is generally involved in transport of the protein to cell organelles where it is processed for secretion outside the cell.

The first conserved domain region comprises amino acids (amino acids 29 to 213 of SEQ ID NO: 2) that are most similar to a thrombospondin N-terminal-like domain. Thrombospondins are a family of related, adhesive glycoproteins, which are synthesized, secreted and incorporated into the (ECM) of a variety of cells, including alpha granules of platelets following thrombin activation and endothelial cells. They interact with a number of blood coagulation factors and anticoagulant factors, and are involved in cell adhesion, platelet aggregation, cell proliferation, angiogenesis, tumor metastasis, vascular smooth muscle growth and tissue repair. The first conserved domain also comprises amino acids (amino acids 82 to 206; amino acids 98 to 209 of SEQ ID NO: 2) that are similar to a Laminin G-like domain. Laminin G-like (LamG) domains usually are Ca²⁺ mediated receptors that can have binding sites for steroids, β1-integrins, heparin, sulfatides, fibulin-1, and α-dystroglycans. Proteins that contain LamG domains serve a variety of purposes, including signal transduction via cell-surface steroid receptors, adhesion, migration and differentiation through mediation of cell adhesion molecules.

Studies show that NELL1 signaling involves an integrin-related molecule and tyrosine kinases that are triggered by NELL1 binding to a NELL1 specific receptor and a subsequent formation of an extracellular complex. As thus far understood, in human NELL1 (hNELL1), the laminin G domain comprises about 128 amino acid residues that show a high degree of similarity to the laminin G domain of extracellular matrix (ECM) proteins; such as human laminin α3 chain (hLAMA3), mouse laminin α3 chain (mLAMA3), human collagen 11 α3 chain (hCOLA1), and human thrombospondin-1 (hTSP1). This complex facilitates either activation of tyrosine kinases, inactivation of tyrosine phosphatases, or intracellular recruitment of tyrosine-phosphorylated proteins. The ligand bound integrin (cell surface receptors that interact with ECM proteins such as, for example, laminin 5, fibronectin, vitronectin, TSP1/2) transduces the signals through activation of the focal adhesion kinase (FAK) followed by indirect activation of the Ras-MAPK cascade, and then leads to osteogenic differentiation through Runx2; the laminin G domain is believed to play a role in the interaction between integrins and a 67 kDa laminin receptor (Shen et al. (2012) J Cell Biochem 113:3620-3628).

The second conserved domain (amino acids 273 to 331 of SEQ ID NO: 2) and seventh conserved domain (amino acids 701 to 749 of SEQ ID NO: 2) are similar to von Willebrand factor type C (VWC) domains, also known as chordin-like repeats. An additional VWC domain is also found from amino acid residues 634 to 686 of SEQ ID NO: 2. VWC domains occur in numerous proteins of diverse functions and have been associated with facilitating protein oligomerization.

The third conserved domain (amino acids 434 to 466 of SEQ ID NO: 2), fourth conserved domain (amino acids 478 to 512 of SEQ ID NO: 2), fifth conserved domain (amino acids 549 to 586 of SEQ ID NO: 2), and sixth conserved domain (amino acids 596 to 627 of SEQ ID NO: 2) are similar to a calcium-binding EGF-like domain. Calcium-binding EGF-like domains are present in a large number of membrane-bound and extracellular (mostly animal) proteins. Many of these proteins require calcium for their biological function. Calcium-binding sites have been found to be located at the N-terminus of particular EGF-like domains, suggesting calcium-binding may be crucial for numerous protein-protein interactions. Six conserved core cysteines form three disulfide bridges as in non-calcium-binding EGF domains whose structures are very similar. The calcium-binding EGF-like domains of NELL1 bind protein kinase C beta, which is typically involved in cell signaling pathways in growth and differentiation.

The nel-like 1 isoform 2 precursor transcript variant (set forth in GenBank Acc. No. NM_201551 and SEQ ID NO: 3) lacks an alternate in-frame exon compared to variant 1. The resulting isoform 2 (set forth in SEQ ID NO: 4), which has the same N- and C-termini as isoform 1 but is shorter compared to isoform 1, has six conserved regions including a TSPN domain/LamG superfamily domain (amino acids 29 to 213 of SEQ ID NO: 4); VWC domains (amino acids 273 to 331 of SEQ ID NO: 4; amino acids 654 to 702 of SEQ ID NO: 4); and calcium-binding EGF-like domains (amino acids 478 to 512 of SEQ ID NO: 4; amino acids 434 to 466 of SEQ ID NO: 4; amino acids 549 to 580 of SEQ ID NO: 4).

NELL1 and its orthologs are found across several species including Homo sapiens (man), Bos taurus (cow; the nucleic acid sequence of which is set forth in GenBank Acc. No. XM_002699102 and the amino acid sequence is set forth in SEQ ID NO: 19), Equus caballus (horse; the nucleic acid sequence of isoforms 1 and 2 are set forth in GenBank Acc. Nos. XM_001504986 and XM_001504987, respectively, and in SEQ ID NO: 5 and 7, respectively; the amino acid sequences are set forth in SEQ ID NO: 6 and 8, respectively), Macaca mulatta (rhesus monkey; the nucleic acid sequence of isoforms 1, 2, 3, and 4 are set forth in GenBank Acc. Nos. XM_002799606, XM_001092428, XM_001092540, and XM_001092655, respectively), Mus musculus (mouse; the nucleic acid sequence of which is set forth in GenBank Acc. No. NM_001037906 and in SEQ ID NO: 9; the amino acid sequence of which is set forth in SEQ ID NO: 10), Rattus norvegicus (rat; the nucleic acid sequence of which is set forth in GenBank Acc. No. NM_031069 and in SEQ ID NO: 11; the amino acid sequence of which is set forth in SEQ ID NO: 12), Pan troglodytes (chimpanzee; the nucleic acid sequence of which is set forth in GenBank Acc. No. XM_508331.2), Felis catus (cat; the amino acid sequences of isoform 1 and 2 are set forth in GenBank Acc. Nos. XP_003993117.1 and XP_003993118.1, and SEQ ID NOs: 13 and 14, respectively, Canis lupis familiaris (dog; the amino acid sequence is set forth in GenBank Acc. No. XP 534090 and SEQ ID NO: 15), and Ovis aries (sheep; the amino acid sequence is set forth in GenBank Acc. No. XP_004019490 and SEQ ID NO: 16).

NELL1 is an extracellular protein that is abundant during mammalian fetal development and mediates pathways encompassing many signaling and structural proteins, that are essential for promoting and balancing tissue growth and maturation (Matsuhashi S et al. 1995 Dev Dyn 203:2012-22; Ting K et al. 1999 J Bone Miner Res 14:80-9; Zhang X et al. 2002 J Clin Invest 110:861-870; Desai J et al. 2006 Hum Mol Genet 15(8):1329-1341; Li C et al. 2017 Am J Pathol 187(5):963-972, doi: 10.1016/j.ajpath.2016.12.026; Li C et al. 2018 Am J Pathol 188(2):392-403, doi:10.1016/j.apath.2017.09.020). A rapidly increasing body of published studies on in vitro and in vivo (small and large animals) models have demonstrated NELL1's ability to restore and regenerate functional tissue after acute injury in bone (Lu S S et al. 2007 Spine J 7(1):50-60; Aghaloo T et al. 2007 Mol Ther 15(10):1872-1880; Xue J et al. 2011 Bone 48(3):485-95; Aghaloo T et al. 2006 Am J of Path 169(3):903-915; Cowan C M et al. 2006 Bone 38:48-58), cartilage (Lee Metal. 2010 Tissue Eng Part A 16(5):1791-1800; Siu R K et al. 2012 Tissue Eng Part A 18(3-4):252-61, doi: 10.1089/ten.TEA.2011.0142; Pakvasa M et al. 2017 Genes and Diseases 4:127-137), skin and muscle (Mitchell D et al. 2012 Journal of the American Academy of Dermatology 66(4): Supplement 1, Page AB3; Turner N et al. 2013 Cells, Tissues and Organs 198(4):249-265). Moreover, human genetic studies and small animal models have established the role of NELL1 in maintaining the balance of cell growth vs. differentiation and tissue formation vs. breakdown, especially in organs where rapid continual breakdown and renewal are necessary to maintain function—bone, epithelial linings of the esophagus, and gastrointestinal tract (James A W et al. 2015 Nature Communications 6:7362, doi:10.1038/ncomms8362; Jin Z et al. 2007 Oncogene doi:10.1038/sj.onc.1210461; Mori Y et al. 2006 Gastroenterology 131:797-808; Nakamura R et al. 2014 J. Biol. Chem doi:10.1074/jbc.M113.507020). During early development, NELL1 regulates the production of many components of the extracellular matrix (ECM) which collectively serve as an architectural framework and communication highway to mediate new tissue formation.

Applying the NELL1 pathway to treat MA is a comprehensive approach to addressing MA and sets NELL1 apart from other biologics. Without being bound by any theory or mechanism of action, it is believed that NELL1 treats MA by addressing both muscle breakdown and formation pathways. Specifically, it is believed to reduce potent pro-inflammatory molecules that trigger protein degradation and subsequent muscle loss. NELL1 is also believed to promote muscle formation and maintenance via the production of certain extracellular matrix proteins that mediate regeneration and impart muscle function and strength and in some cases, through the promotion of muscle precursor cells to maturity.

The presently disclosed methods utilize a NELL1 polypeptide or a nucleic acid molecule encoding the same to treat muscle atrophy. As used herein and in the claims, a “NELL1 polypeptide” refers to a naturally occurring NELL1 polypeptide of any species, as well as variants and fragments of such naturally occurring polypeptides as described herein.

A peptide, polypeptide, or protein is a sequence of subunit amino acids, amino acid analogs, or peptidomimetics. A peptidomimetic is a small protein-like chain designed to mimic a peptide. A peptidomimetic typically arises from modification of an existing peptide in order to alter the molecule's properties.

A peptide, polypeptide or protein can also be amino acid polymers in which one or more amino acid residue is an artificial chemical analogue of a corresponding naturally occurring amino acid, as well as to naturally-occurring amino acid polymers. A polypeptide, peptide or protein is inclusive of modifications including, but not limited to, glycosylation, lipid attachment, sulfation, gamma-carboxylation of glutamic acid residues, hydroxylation, phosphorylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing events and events brought about by human manipulation which do not occur naturally. Circular, branched and branched circular polypeptides may be synthesized by non-translation natural processes and by entirely synthetic methods, as well.

NELL1 has regenerative properties. The regeneration of tissue refers to the process of renewal and growth of cells and extracellular matrix components within a particular tissue that results in the production of tissue that has a cellular component and architecture that allows for the normal functions of the particular tissue type. A NELL1 peptide, NELL1 polypeptide, or NELL1 protein is a naturally-occurring NELL1 protein, or a variant or fragment thereof that retains the ability to regenerate or maintain healthy muscle tissue. Thus, an active NELL1 variant or fragment retains the ability to build muscle (e.g., increase in muscle mass, increased fusion of satellite cells, increase in muscle protein synthesis), enhance muscle activity (e.g., contractility, strength), and/or prevent muscle loss (e.g., muscle protein degradation) or activity. In some embodiments, the NELL1 polypeptide exhibits any one of the activities selected from the group consisting of: stimulation of ECM production (e.g., through the upregulation of at least one of tenascins, proteoglycans, elastin, glycosaminoglycans, including epidermal hyaluronic acid, and collagens), reduction in the levels of inflammatory mediators (e.g., IL-1β and IL-8), and reduction in the levels of matrix metalloproteinases (e.g., MMP1).

In other embodiments, the NELL1 polypeptide can also exhibit at least one of the activities selected from the group consisting of: binding to PKC-beta, stimulation of differentiation of a precursor cell (e.g., skeletal satellite cell, osteoblast precursor, perivascular stem cell) to maturity, and stimulation of angiogenesis. To determine whether a polypeptide exhibits any one of these activities, any method known in the art useful for measuring these activities can be used.

Suitable assays for determining if a given polypeptide can stimulate ECM production and reduce the levels of inflammatory mediators or MMPs include assays that measure transcript levels (e.g., quantitative polymerase chain reaction) or levels of the protein (e.g., enzyme-linked immunoassay) directly or indirectly (by measuring the activity of the protein), including those that are described elsewhere herein.

Suitable assays for assessing the binding of NELL1 to PKC beta is described in e.g., Kuroda et al. (1999) Biochem Biophys Res Comm 265:752-757. For example, protein-protein interactions can be analyzed by using the yeast two-hybrid system. Briefly, a NELL1 polypeptide can be fused with GAL4 activating domain and the regulatory domain of PKC can be fused with the GAL4 DNA-binding domain.

In other embodiments, the NELL1 polypeptide stimulates the fusion of skeletal satellite cells with existing muscle fibers. The nuclei count of muscle fibers can be assessed histologically using methods known in the art, including those described elsewhere herein.

The NELL1 polypeptide may be a naturally-occurring (i.e., wild-type) NELL1 protein or an active variant or fragment thereof. Naturally refers to as found in nature; wild-type; innately or inherently. A naturally-occurring NELL1 polypeptide may be purified from a natural source or may be a polypeptide that has been recombinantly or synthetically produced that has the same amino acid sequence as a NELL1 polypeptide found in nature.

A polynucleotide can be a singular nucleic acid, as well as plural nucleic acids, and refers to a nucleic acid molecule or construct, e.g., messenger RNA (mRNA), complementary DNA (cDNA), or plasmid DNA (pDNA). A polynucleotide (e.g., nucleic acid molecule) can be single-stranded or double-stranded, linear or circular and can be comprised of DNA, RNA, or a combination thereof. A polynucleotide (e.g., nucleic acid molecule) can comprise a conventional phosphodiester bond or a non-conventional bond (e.g., an amide bond, such as found in peptide nucleic acids (PNA)). A nucleic acid can be any one or more nucleic acid segments, e.g., DNA or RNA fragments, present in a polynucleotide. The polynucleotide (e.g., nucleic acid molecule) can contain modified nucleic acids, such as phosphorothioate, phosphate, ring atom modified derivatives, and the like. The polynucleotide (e.g., nucleic acid molecule) can be a naturally occurring polynucleotide (i.e., one existing in nature without human intervention), a recombinant polynucleotide (i.e., one existing with human intervention), or a synthetically derived polynucleotide.

An isolated material can refer to a nucleic acid, peptide, polypeptide, or protein, which is: (1) substantially or essentially free from components that normally accompany or interact with it as found in its naturally occurring environment. Substantially free or essentially free refer to considerably or significantly free of, or more than about 95% free of, or more than about 99% free of. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state. For example, a naturally occurring nucleic acid becomes an isolated nucleic acid if it is altered, or if it is transcribed from DNA that has been altered, by means of human intervention performed within the cell from which it originates. See, for example, Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (for example, a promoter) becomes isolated if it is introduced by non-naturally occurring means to a locus of the genome not native to that nucleic acid.

Fragments and variants of native (i.e., naturally-occurring) NELL polypeptides can be employed in the various methods and compositions of the invention. A fragment is intended a portion of a polynucleotide or a portion of a polypeptide. Fragments of a polynucleotide may encode polypeptide fragments that retain the biological activity of the native polypeptide. A fragment of a polynucleotide that encodes a biologically active portion of a NELL1 polypeptide will encode at least 15, 25, 30, 50, 100, 150, 200, 250, 300, 350, 400, 450, 500, 550, 600, 650, 700, 750, or 800 contiguous amino acids, or up to the total number of amino acids present in a full-length NELL1 polypeptide. In certain embodiments, the NELL1 fragment is 610 amino acids in length.

A fragment of a native NELL1 polypeptide can be prepared by isolating a portion of a polynucleotide encoding the portion of the NELL1 polypeptide and expressing the encoded portion of the polypeptide (e.g., by recombinant expression in vitro). Polynucleotides that encode fragments of a NELL1 polypeptide can comprise nucleotide sequences comprising at least 15, 20, 50, 75, 100, 150, 200, 250, 300, 350, 400, 450, 500, 550, 600, 650, 700, 800, 900, 1000, 1100, 1200, 1300, 1400, 1500, 1600, 1700, 1800, 1900, 2000, 2100, 2200, 2300, or 2400 contiguous nucleotides, or up to the number of nucleotides present in a full-length NELL1 nucleotide sequence. In some embodiments, the fragment lacks the first amino acid residue, or the first 2, 3, 4, 5, 6, 7, 8, 9, 10, 20, 21, 22, 23, 24, 25, 26, 27, 28, 29, 30, 35, 40, or 45 amino acid residues from the amino terminal end of the NELL1 protein. In some embodiments, the fragment lacks the last 5, 10, 15, 20, 25, 30, 35, 40, 45, 50, 55, 60, 65, 70, 75, 80, 85, 90, 95, 100, 110, 115, 120, 125, 130, 135, 140, 145, 150, 155, 160, 165, 170, 175, 180, 185, 190, 195, 200, 205, 210, 220, 230, 240, 250, 260 or more amino acid residues. In certain embodiments, the fragment of a NELL1 protein lacks the most carboxy-terminal 179 amino acid residues from the end of the protein. In other embodiments, the NELL1 protein fragment lacks the first two amino acid residues from the amino terminal end and the last 179 amino acid residues from the carboxy terminal end of the protein. In some embodiments, the NELL1 protein fragment has 610 amino acid residues.

Removal of 179 amino acid residues from the carboxy-terminus of the Equus caballus NELL1 isoform 1 protein unexpectedly provided a higher yield and easier purification during manufacture of the protein (U.S. Patent Application Publication No. 2018/0057550). Without being bound by any theory or mechanism of action, it is believed that the removal of the carboxy-terminal domains led to decreased formation of aggregates of the protein. Although NELL1 protein naturally oligomerizes into trimers, which are functional, aggregates of NELL1 protein refer to large, higher-ordered macromolecular complexes that prevent or reduce the function of the protein or make the protein products difficult to extract and purify. The NELL1 protein lacking the C-terminal 179 amino acid residues is also unexpectedly more efficacious than full-length NELL1 protein in horse body wound healing studies and fibroblast wound scratch assays. Thus, in specific embodiments, the NELL1 protein fragment lacks the last 179 amino acid residues from the carboxy terminus. In some of these embodiments, the NELL1 protein fragment also lacks the first two amino acid residues from the amino terminus. The sequence of this horse NELL1 fragment is set forth in SEQ ID NO: 18. In other embodiments, the NELL1 protein fragment lacks the first 21 amino acid residues from the amino terminus and the last 179 amino acid residues from the carboxy terminus. The sequence of this human NELL1 fragment is set forth in SEQ ID NO: 17, also referred to herein as NV1. In certain embodiments, the NELL1 protein fragment lacks at least one of the two carboxy-terminal VWC domains (located at amino acid residues 634-686 and 701-749 of SEQ ID NO: 2). In some of these embodiments, the NELL1 protein fragment lacks both of these carboxy-terminal VWC domains.

In those embodiments wherein a NELL1 protein fragment lacks at least one C-terminal VWC domain, the NELL1 protein fragment exhibits at least one of the following characteristics: enhanced efficacy in tissue regeneration and/or promotion of wound healing, enhanced prevention of tissue loss (e.g., skeletal muscle loss), easier purification, higher yield, less aggregate formation, and enhanced efficacy in fibroblast migration and/or proliferation, when compared to its respective full-length NELL1 protein. An easier purification includes a purification process whereby a single polypeptide species is substantially separated from other polypeptide species or a natural or synthetic milieu comprising the single polypeptide species and other polypeptide species that comprises fewer steps required for substantial separation or wherein the time required for at least one of the steps in the separation is reduced. An easier purification also refers to a purification process which results in a higher yield of the substantially purified or separated polypeptide species when compared to its respective full-length protein. The terms “substantially purified” or “substantially separated” when used in reference to a single polypeptide species refers to a level of purification whereby the single polypeptide species represents at least about 70% of a total population of polypeptide species within a sample, including but not limited to at least about 75%, 80%, 85%, 86%, 87%, 88%, 89%, 90%, 91%, 92%, 93%, 94%, 95%, 96%, 97%, 98%, 99%, or greater of a total population of polypeptide species within a sample. A yield of a protein product from a purification process refers to the overall concentration of the polypeptide within a solution. The higher the concentration of the polypeptide within the solution, the more yield is obtained. If a polypeptide is present within a solution at <0.1 μg/μl, the protein is considered difficult to produce and purify. Thus, in some embodiments, a NELL1 protein fragment that lacks at least one C-terminal VWC domain exhibits the ability to be purified using conventional purification means known in the art, such as those methods described elsewhere herein, to a concentration greater than 0.1 μg/μ1. In some of these embodiments, a NELL1 protein fragment has the ability to be purified using conventional purification means known in the art to a concentration of about 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30 μg/μl, or greater. In certain embodiments, a NELL1 protein fragment lacking at least one C-terminal VWC domain exhibits both a higher yield and a greater purity as compared to its respective full-length NELL1 protein following a purification process.

Variant sequences have a high degree of sequence similarity. For polynucleotides, conservative variants include those sequences that, because of the degeneracy of the genetic code, encode the amino acid sequence of a NELL1 polypeptide. Variants such as these can be identified with the use of well-known molecular biology techniques, such as, for example, polymerase chain reaction (PCR) and hybridization techniques. Variant polynucleotides also include synthetically derived nucleotide sequences, such as those generated, for example, by using site-directed mutagenesis. In some embodiments, the variant polynucleotide still encodes a NELL1 polypeptide or a fragment thereof. Generally, variants of a particular polynucleotide will have at least about 60%, 65%, 70%, 75%, 80%, 85%, 90%, 91%, 92%, 93%, 94%, 95%, 96%, 97%, 98%, 99% or more sequence identity to that particular polynucleotide, when compared over the full length of the variant, as determined by sequence alignment programs and parameters described elsewhere herein.

Variants of a particular polynucleotide can also be evaluated by comparison of the percent sequence identity between the polypeptide encoded by a variant polynucleotide and the polypeptide encoded by the reference polynucleotide. Thus, variants include, for example, polynucleotides that encode a polypeptide with a given percent sequence identity to a native NELL1 polypeptide. Percent sequence identity between any two polypeptides can be calculated using sequence alignment programs and parameters described herein. Where any given pair of polynucleotides is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity.

A variant polypeptide is a polypeptide derived from the native polypeptide by deletion (so-called truncation) or addition of one or more amino acids to the N-terminal and/or C-terminal end of the native polypeptide; deletion or addition of one or more amino acids at one or more sites in the native polypeptide; or substitution of one or more amino acids at one or more sites in the native polypeptide. The activity of variant NELL1 polypeptides can be assessed using the methods disclosed herein to determine if the variant is biologically active. Such variants may result from, for example, genetic polymorphism or from human manipulation. Biologically active variants of a native NELL1 polypeptide will have at least about 60%, 65%, 70%, 75%, 80%, 85%, 90%, 91%, 92%, 93%, 94%, 95%, 96%, 97%, 98%, 99% or more sequence identity to the amino acid sequence for the native polypeptide, when compared over the full length of the variant, as determined by sequence alignment programs and parameters described elsewhere herein. A biologically active variant of a polypeptide may differ from that polypeptide by as few as 1-15 amino acid residues, as few as 1-10, such as 6-10, as few as 5, as few as 4, 3, 2, or even 1 amino acid residue.

Biologically active variants of the NELL1 fragments disclosed herein (i.e., those lacking at least one of the two VWC domains at the carboxy terminus of NELL1) are also contemplated herein and may have at least about 60%, 65%, 70%, 75%, 80%, 85%, 90%, 91%, 92%, 93%, 94%, 95%, 96%, 97%, 98%, 99% or more sequence identity to the amino acid sequence for the active NELL1 fragment (e.g., SEQ ID NO: 17 or 18).

Polypeptides may be altered in various ways including amino acid substitutions, deletions, truncations, and insertions. Methods for such manipulations are generally known in the art. For example, amino acid sequence variants of native NELL1 polypeptides can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the polypeptide of interest may be found in the model of Dayhoff et al. (1978) Atlas of Protein Sequence and Structure (Natl. Biomed. Res. Found., Washington, D.C.). Conservative substitutions, such as exchanging one amino acid with another having similar properties, may be preferable.

Generally, the mutations made in the polynucleotide encoding the variant NELL1 polypeptide should not place the sequence out of reading frame, and/or create complementary regions that could produce secondary mRNA structure. See, EP Patent Application Publication No. 75,444.

Variant NELL1 polynucleotides and polypeptides also encompass sequences and polypeptides derived from a mutagenic and recombinogenic procedure such as DNA shuffling. With such a procedure, one or more different NELL1 coding sequences can be manipulated to create peptides that can be evaluated to determine if it retains NELL1 activity. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Variant NELL1 polynucleotides and polypeptides also encompass sequences and polypeptides derived from gene editing systems, such as CRISPR/Cas system.

Sequence identity in the context of two polynucleotides or polypeptide sequences makes reference to the residues in the two sequences that are the same when aligned for maximum correspondence over a specified comparison window. When percentage of sequence identity is used in reference to polypeptides it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have sequence similarity or similarity. Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

Percentage of sequence identity is the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3; % identity and % similarity for an amino acid sequence using GAP Weight of 8 and Length Weight of 2, and the BLOSUM62 scoring matrix; or any equivalent program thereof. An equivalent program is any sequence comparison program that, for any two sequences in question, generates an alignment having identical nucleotide or amino acid residue matches and an identical percent sequence identity when compared to the corresponding alignment generated by GAP Version 10.

The NELL1 polypeptide may be made synthetically, i.e. from individual amino acids, or semi-synthetically, i.e. from oligopeptide units or a combination of oligopeptide units and individual amino acids. Alternatively, the protein can be synthesized in a cell-free in vitro translation system, such as a wheat germ cell-free system (see, for example, Madin et al. (2000) Proc. Natl. Acad. Sci. U.S.A. 97(2):559-564; Sawasaki et al. (2000) Nucleic Acids Symp Ser 44:9-10; Sawasaki et al. (2002) Proc. Natl. Acad. Sci. U.S.A. 99(23):14652-14657; and Endo and Sawasaki (2003) Biotechnol. Adv. 21(8):695-713). Suitable methods for synthesizing proteins are described by Stuart and Young in “Solid Phase Peptide Synthesis,” Second Edition, Pierce Chemical Company (1984), Solid Phase Peptide Synthesis, Methods Enzymol., 289, Academic Press, Inc, New York (1997).

The NELL1 polypeptide may also be prepared by methods that are well known in the art. One such method includes isolating or synthesizing DNA encoding the NELL1 polypeptide, and producing the recombinant protein by expressing the DNA, optionally in a recombinant vector, in a suitable host cell. Suitable methods for synthesizing DNA are described by Caruthers et al. (1985) Science 230:281-285; and DNA Structure, Part A: Synthesis and Physical Analysis of DNA, Lilley, D. M. J. and Dahlberg, J. E. (Eds.), Methods Enzymol., 211, Academic Press, Inc., New York (1992).

In some embodiments of the presently disclosed methods, a nucleic acid molecule encoding a NELL1 polypeptide is administered to a subject in need thereof in order to treat muscle atrophy. As used herein, the terms “encoding” or “encoded” when used in the context of a specified nucleic acid mean that the nucleic acid comprises the requisite information to direct translation of the nucleotide sequence into a specified polypeptide.

In some embodiments of the presently disclosed methods, the NELL1 nucleic acid molecule is operably linked to at least one regulatory element. A regulatory element is a nucleic acid sequence(s) capable of effecting the expression of nucleic acid(s), or the peptide or protein product thereof. Non-limiting examples of regulatory elements include promoters, enhancers, polyadenylation signals, transcription or translation termination signals, ribosome binding sites, or other segments of DNA where regulatory proteins, such as, but not limited to, transcription factors, bind preferentially to control gene expression and thus protein expression.

Regulatory elements may be operably linked to the nucleic acids, peptides, or proteins of the described invention. When two or more elements are operably linked, there exists a functional linkage between the elements. For example, when a promoter and a protein coding sequence are operably linked, the promoter sequence initiates and mediates transcription of the protein coding sequence. The regulatory elements need not be contiguous with the nucleic acids, peptides, or proteins whose expression they control as long as they function to direct the expression thereof. Thus, for example, intervening untranslated yet transcribed sequences may be present between a promoter sequence and a nucleic acid of the described invention and the promoter sequence may still be considered operably linked to the coding sequence.

In certain embodiments, the NELL1 nucleic acid molecule is a recombinant expression cassette or is part of an expression system. The term “recombinant expression cassette” refers to a nucleic acid construct, generated recombinantly or synthetically, with a series of specified nucleic acid elements which permit transcription of a particular nucleic acid (e.g., protein coding sequence) in a host cell. The recombinant expression cassette can be incorporated into a plasmid, chromosome, mitochondrial DNA, virus, or nucleic acid fragment. Typically, the recombinant expression cassette portion of an expression vector includes, among other sequences, a nucleic acid to be transcribed, a promoter, and a transcription termination signal such as a poly-A signal.

The expression cassette or cloning vector can be generated using molecular biology techniques known in the art and utilizing restriction enzymes, ligases, recombinases, and nucleic acid amplification techniques such as polymerase chain reaction that can be coupled with reverse transcription.

In some embodiments, the NELL1 polypeptide is produced using a cell-free expression system such as the wheat germ in vitro translation system.

In some embodiments, the NELL1 nucleic acid molecule is in a host cell that can be used for propagation of the nucleic acid molecule or for expression of the NELL1 polypeptide and subsequent isolation and/or purification. A host cell is any cell that contains a heterologous nucleic acid molecule. A heterologous polypeptide or nucleotide sequence is a polypeptide or a sequence that originates from a different species, or if from the same species, is substantially modified from its native form in composition and/or genomic locus by deliberate human intervention. The host cell typically supports the replication and/or expression of the vector. Host cells may be prokaryotic cells such as, but not limited to, Escherichia coli, or eukaryotic cells such as, but not limited to, yeast, insect, amphibian, plant (e.g., Nicotiana tabacum (tobacco), Oryza sativa (rice), Arabidopsis thaliana (cress)), or mammalian cells. The term as used herein means any cell which may exist in culture or in vivo as part of a unicellular organism, part of a multicellular organism, or a fused or engineered cell culture. A cloning host cell is a host cell that contains a cloning vector.

A recombinant cell or vector is one that has been modified by the introduction of a heterologous nucleic acid or the cell that is derived from a cell so modified. Recombinant cells express genes that are not found in identical form within the native (non-recombinant) form of the cell or express native genes that are otherwise abnormally expressed, under-expressed or not expressed at all as a result of deliberate human intervention. The alteration of a cell or vector by naturally occurring events (e.g., spontaneous mutation, natural transformation transduction/transposition), such as those occurring without deliberate human intervention, does not result in a recombinant cell or vector.

The NELL1 nucleic acid molecule can be introduced into a host cell for propagation of production of NELL1 using any method known in the art, including transfection, transformation, or transduction, so long as the nucleic acid molecule gains access to the interior of the cell. The insertion or introduction of a nucleic acid into a cell refers to transfection or transformation or transduction and includes the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The NELL1 nucleic acid molecule can be introduced into a host cell to allow for stable transformation or transient transformation. Stable transformation is intended to mean that the nucleotide construct introduced into a cell integrates into a genome of the cell. Transient transformation is intended to mean that a polynucleotide is introduced into the cell and does not integrate into a genome of the cell.

The NELL1 polypeptide can be administered by a cell based gene therapy. For example, autologous, allogeneic or xenogeneic donor cells are genetically modified in vitro to express and secrete the NELL1 polypeptide. The genetically modified donor cells are then subsequently implanted into the subject in need of delivery of the NELL1 polypeptide in vivo. Examples of suitable cells include, but are not limited to, skeletal satellite cells, induced pluripotent stem cells, or adult mesenchymal stem cells.

### III. Methods of Treatment

The presently disclosed methods involve the treatment of muscle atrophy in a subject in need thereof. The terms “subject”, “individual”, and “patient” are used interchangeably to refer to a member of a species that comprises skeletal muscle and cardiac muscle. In certain embodiments, the subject is a mammal, including but not limited to, mouse, rat, cat, goat, sheep, horse, hamster, ferret, pig, dog, platypus, guinea pig, rabbit and a primate, such as, for example, a monkey, ape, or human. In some of these embodiments, the subject is a human, cat, dog, or a horse, such as a racehorse.

Muscle atrophy may refer to a disease or condition characterized by the decrease in the mass of a muscle, fiber size, cross-sectional area, or other muscle characteristic in a subject and/or a progressive weakening and degeneration of muscle tissue. A decrease in the mass of the muscle is usually accompanied with a weakening of the muscles (i.e. decreasing muscle function). In some embodiments, muscle atrophy may refer to a decrease in a muscle characteristic (e.g., mass) of at least about 10%, at least about 15%, at least about 20%, at least about 25%, at least about 30%, at least about 35%, at least about 40%, at least about 45%, at least about 50%, or more relative to the same muscle tissue in a healthy/normal subject (i.e. a control subject) or population of healthy/normal individuals (e.g., relative to average, medium, or minimum threshold values) or relative to a recorded or estimated baseline value in the subject. Symptoms of muscle atrophy can include impaired muscle coordination, smaller appearance of muscles, muscle fatigue, muscle weakness, and impaired balance. These symptoms, such as muscle strength, may be measured by an appropriate test known in the art. Muscle atrophy can be physiologic, pathologic, or neurogenic. Physiologic muscle atrophy is caused by inadequate use of the muscles due to decreased activity, immobilization (for example, secondary to an injury or paralysis), or lack of gravity. Pathologic muscle atrophy has an underlying pathologic cause, such as aging, starvation, malnutrition, anorexia, and various diseases such as those that are treated with long-term corticosteroid therapy, and cancer. Neurogenic muscle atrophy results from an injury or disease of a nerve that connects to the muscle. Examples of neurogenic muscle atrophy include atrophy occurring due to disorders such as amyotrophic lateral sclerosis, carpal tunnel syndrome, Guillain-Barré syndrome, nerve damage, spinal cord injury, and polio.

Muscle atrophy may refer to atrophy of skeletal muscle tissue or cardiac muscle tissue. Cardiac muscle atrophy is often characterized by ventricular wall thinning and a decrease in cardiomyocyte cell size. Cardiac atrophy can be caused by a physiological response to chronically reduced cardiac workload or to complex inflammatory disease milieus or chronic systemic inflammation. Symptoms of cardiac muscle atrophy include an irregular heartbeat, heart failure, a heart valve problem, or other complications.

Muscle atrophy in an in vitro context may refer to muscle cell shriveling, muscle cell death, etc. Muscle atrophy may occur as the result of any number of stimuli or conditions, including, but not limited to fasting, cachexia, diabetes, immobilization, disuse, muscular dystrophy, other myopathies etc. Muscle atrophy results from an imbalance between protein synthesis and protein degradation. The term “muscle atrophy” encompasses sarcopenia (loss of muscle tissue due to aging), cachexia, and muscle wasting. Muscle atrophy may be diagnosed according to methods known in the art, which may include imaging (e.g., CT scanning, MRI) or by biomarker analysis.

Treating a subject refers to the administering of the NELL1 polypeptide or a nucleic acid molecule encoding the same to a subject for a therapeutic or prophylactic purpose. Administration may include any method of delivery of the NELL1 polypeptide or nucleic acid molecule encoding the same into the subject's system or to a particular region in or on the subject (i.e., systemic or local administration). In some embodiments, the NELL1 polypeptide or nucleic acid molecule encoding the same may be administered to a subject for the treatment of diseases, conditions, symptoms, and/or afflictions which can be cured, alleviated, prevented, delayed, managed or improved by increasing muscle mass, strength, power and/or function. Treatments may include treatments of diseases or conditions characterized by a decreased muscle mass, strength, power, or function in a subject, relative to a healthy/normal subject (i.e. a control subject) or population of healthy/normal individuals (e.g., relative to average, medium, or minimum threshold values) or relative to a recorded or estimated baseline value in the subject.

Treatment of muscle atrophy with a NELL1 polypeptide or nucleic acid molecule encoding the same can result in a partial or complete recovery of muscle tissue, function, and/or strength or a partial or complete prevention of muscle atrophy or symptoms associated therewith. Thus, treatment of muscle atrophy with a NELL1 polypeptide or nucleic acid molecule encoding the same can result in at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, or more recovery of muscle tissue (e.g., area), function, and/or strength in a subject experiencing muscle atrophy or the onset of muscle atrophy has been delayed or the symptoms lessened through prophylactic treatment with a NELL1 polypeptide or nucleic acid molecule encoding the same.

In some embodiments, the disease/condition treated with a NELL1 polypeptide or a nucleic acid molecule encoding the same may be muscle atrophy or diseases or conditions related thereto or associated therewith. Other diseases or conditions which are suitable for treatment by increasing muscle mass, strength, power and/or function are well understood by those of ordinary skill in the art. Treatable diseases or conditions may be either idiopathic or secondary to other conditions, such as cancer, disuse, immobilization, bed rest, injury, surgery, etc. Treatment may include prophylactic treatment of subjects not presently suffering from a disease, symptom, condition, and/or affliction. In some embodiments, subjects who are at risk of developing a disease, symptom, condition, and/or affliction (e.g., have an increased likelihood relative to a general population of subjects) or who show mild or moderate signs or symptoms of a disease, condition, or affliction (i.e. and are at risk for progressing to a more severe state) may be treated. Subjects suitable for prophylactic treatment can include subjects who may receive a benefit from treatment. Suitable subjects may include subjects at risk of a disease, condition, or affliction due to innate factors (e.g. genetic/hereditary) and/or external factors (e.g., subjects who suffered a recent injury). Subjects who are not presently at a risk but who are expected to be at risk in the future (e.g., subjects undergoing a planned surgery) may also be suitable subjects for prophylactic treatment.

Subjects in need of treatment of muscle atrophy include those that exhibit symptoms of muscle atrophy (e.g., muscle fatigue and weakness, loss of muscle coordination, reduction in size of limbs) and those at risk of developing muscle atrophy. Subjects at risk of developing muscle atrophy include those that are malnourished due to poor diets (e.g., low protein diet) or as the result of a medical condition that impairs the body's ability to absorb nutrients (e.g., irritable bowel syndrome, celiac disease), immobilized subjects, aging subjects (e.g., 60 and above), subjects with spinal cord or peripheral nerve injuries, subjects on long-term corticosteroid therapy, cancer patients, and patients with certain muscle wasting diseases such as amyotrophic lateral sclerosis, dermatomyositis, Guillain-Barré syndrome, multiple sclerosis, muscular dystrophy, neuropathy, osteoarthritis, polio, polymyositis, rheumatoid arthritis, and spinal muscular atrophy.

Other subjects that are at high risk for developing muscle atrophy include those that exhibit chronic systemic inflammation that is associated with sustained IL-1β activation. Chronic systemic inflammation is inflammation that is not limited to a particular region of the body and the result of the release of pro-inflammatory cytokines, such as IL-1β, from immune-related cells and the chronic activation of the innate immune system. In contrast, an acute inflammatory response requires constant stimulation to be sustained and is actively terminated once the injurious stimulus has been removed. Chronic systemic inflammation can be caused by untreated causes of acute inflammation, such as an infection by a microbe (e.g., bacteria, fungus, or virus) or injury, an autoimmune disorder, or long-term exposure to irritants, such as certain chemicals or polluted air. Chronic systemic inflammation can be caused by external factors like smoking, alcohol, obesity, and chronic stress or genetic predispositions. Chronic systemic inflammation has been implicated in the pathophysiology of a wide range of seemingly unrelated disorders which underlay a large and varied group of diseases. For example, chronic systemic inflammation is involved in diseases as diverse as cardiovascular diseases, cancers, allergies, obesity, diabetes, digestive system diseases, degenerative diseases, autoimmune disorders, and neurodegenerative diseases such as Alzheimer's disease. Chronic systemic inflammation can be systemic inflammation that occurs for greater than one week, two weeks, three weeks, one month, two months, six months, one year or longer.

In those embodiments wherein chronic systemic inflammation is the result of an infection by a microbe (e.g., bacteria, fungus, or virus), systemic inflammation can initiate during active infection of the microbe and the systemic inflammation continues even after the microbe has been substantially cleared from the subject (e.g., no longer causing symptoms or no longer detectable in a routine diagnostic assay), thus becoming a chronic systemic inflammation.

According to certain embodiments, muscle atrophy is treated in a subject with systemic inflammation resulting from an infection by a microbe. In some embodiments, the subject has an active microbial infection. In other embodiments, the subject no longer has an active infection wherein the microbe has been substantially cleared (e.g., no longer causing symptoms or no longer detectable in a routine diagnostic assay) and the systemic inflammation experienced by the subject is chronic systemic inflammation.

Severe influenza infections have resulted in muscle wasting and myositis associated with inflammation, particularly high levels of the cytokine IL-6. In addition, SARS-CoV-2 infection, which also often elevates IL-6 levels, has been shown to cause weight loss and cachexia (see, e.g., Filippo et al. (2020) Clinical Nutrition, doi.org/10.1016/j.clnu.2020.10.043; and Morley et al. (2020) Journal of Cachexia, Sarcopenia and Muscle 11:863-865) and treatment of SARS-CoV-2-infected transgenic mice expressing human ACE2 with NV1 (a fragment of NELL1 set forth as SEQ ID NO: 17) prevented or reversed the weight loss (data not shown). The weight loss associated with SARS-CoV-2 infection may, at least in part, be attributed to a loss of muscle mass and muscle atrophy and the prevention or reversal of the SARS-CoV-2-induced weight loss by NELL1/NV1 may, at least in part, be due to the treatment of the muscle atrophy and reversal/prevention of damage to skeletal muscle tissue associated with SARS-CoV-2 infection.

In some embodiments wherein muscle atrophy is treated in a subject with systemic inflammation resulting from an infection by a microbe, the microbe is a virus. Muscle dysfunction is common in patients with acute respiratory distress syndrome (ARDS) which can be caused by respiratory viruses, such as influenza A (Radigan et al. (2019) J Immunol 202:484-493) and SARS-CoV-2. Thus, in some embodiments, the virus is a respiratory virus (i.e., virus that infects the upper and/or lower respiratory tract). Non-limiting examples of respiratory viruses include respiratory syncytial virus (RSV), influenza viruses (including influenza A viruses such as H1N1 and H3N2, and influenza B viruses), rhinoviruses, adenovirus, human metapneumovirus (hMPV), parainfluenza virus, and coronaviruses. In some of these embodiments, the virus is a coronavirus (i.e., belonging to the Coronaviridae family). In some embodiments, the virus belongs to the beta group of the Coronaviridae family. In some embodiments, the virus belongs to the gamma group of the Coronaviridae family. In some embodiments, the virus belongs to the delta group of the Coronaviridae family. SARS-CoV-2 shares a highly similar gene sequence and behavior pattern with SARS-CoV (Chan et al., Emerg Microbes Infect. 2020; 9(1):221-236). Both SARS-CoV-2 and SARS-CoV are in the coronavirus family, β-coronavirus genera, lineage B (Chan et al., Id.). In certain embodiments, the coronavirus is a β coronavirus, lineage B (i.e., SARS virus). In particular embodiments, the coronavirus is SARS-CoV-2. SARS-CoV-2 virus can refer to the original virus discovered in Wuhan, China in 2019 (Xu et al., Genomics Proteomics Bioinformatics. 2003 August; 1(3): 226-235; herein incorporated by reference in its entirety), the genome sequence of which is set forth as NCBI Reference Sequence NC_045512.2 (herein incorporated by reference in its entirety) or a variant thereof, including the six types (types I to VI) described by Yang et al. (2020) Proc Natl Acad Sci USA 117(48):30679-30686, which is herein incorporated by reference in its entirety, 20I/501Y.V1, VOC 202012/01 or B.1.1.7 variant, the 20H/501Y.V2 or B.1.351 variant, or the P1 variant. Non-limiting examples of SARS-CoV-2 genome sequences include GenBank Accession No. MN908947.3, NCBI Reference Sequence NC_045512.2, and Global Initiative on Sharing Avian Influenza Data (GISAID) Accession IDs: EPI_ISL 404227, EPI_ISL 404228, EPI_ISL 402132, EPI_ISL 402127, EPI_ISL 402128, EPI_ISL 402129, EPI_ISL 402130, EPI_ISL 402124, EPI_ISL 403963, EPI_ISL 403962, EPI_ISL 402120, EPI_ISL 402119, EPI_ISL 402121, EPI_ISL 402123, EPI_ISL 402125, EPI_ISL 403931, EPI_ISL 403928, EPI_ISL 403930, EPI_ISL 403929, EPI_ISL 403937, EPI_ISL 403936, EPI_ISL 403935, EPI_ISL 403934, EPI_ISL 403933, EPI_ISL 403932, EPI_ISL 404895, EPI_ISL 404253, and EPI_ISL 405839.

The muscle atrophy associated with systemic inflammation from an infection can be atrophy of skeletal muscle tissue and/or cardiac muscle. In certain embodiments, muscle atrophy and/or cachexia associated with systemic inflammation from an infection that is treated with a NELL1 polypeptide or nucleic acid encoding the same comprises skeletal muscle atrophy.

Patients that can also benefit from NELL1 treatment include those with relatively high circulating levels of interleukin-1 beta (IL-1β) when compared to an appropriate control subject (i.e., a healthy/normal subject or population of healthy/normal individuals (e.g., relative to average, medium, or minimum threshold values)) or those with a genetic predisposition for relatively high levels of IL-1β. IL-1β is a pro-inflammatory cytokine that is encoded by the IL1B gene. IL-1β is a potent cytokine that promotes inflammation in the body in response to infection by pathogens or tissue injury. Several types of cells can secrete IL-1β, but it is primarily produced by cells in the immune system such as monocytes and macrophages, which mediate innate immune responses. Although it is currently the best studied cytokine, data suggests that the production, processing, and secretion of IL-1β is regulated in complex ways under different tissue and molecular context or environments (Netea et al. 2010 PLoS Pathogens 6:2 e1000661; Afofina et al. 2015 Immunity 42:991-1004; Dinarello 2018 Immunol Rev 281(1):8-27, doi:10.1111/imr.12621). IL-1β is initially produced as an inactive 31 kDa (269 amino acid) pro-protein (designated pro-IL-1β, the sequence of human pro-IL-1β is set forth in NCBI GenBank Acc. No. NP_000567.1 and SEQ ID NO: 20), which is then cleaved into active forms by different proteolytic enzymes. The two most elucidated processing pathways are mediated by caspase 1 or serine proteases, respectively. Caspase 1 cleaves pro-IL-1β at two distinct sites, producing a minor 25-kDa fragment (of unknown function) and a mature, bioactive 17-kDa IL-1β. This processing pathway is controlled by an inflammasome, a cytosolic multiprotein unit signaling complex activated by a variety of stimuli such as bacteria, compounds, reactive oxygen species, molecular patterns associated with injury or danger (e.g., PAMPs, DAMPs). This activation, in turn, recruits caspase 1 into the inflammasome and leads to the cleavage of inactive pro-IL-1β and formation of the active fragment. There are different inflammasome complexes with NLRP3 as the best characterized complex involved in IL-1β processing and activation. Serine proteases from macrophage and neutrophils can also cleave pro-IL-1β into 21-kDa bioactive fragments. Examples of these types of proteases are proteinase 3 (PR3), elastase, and cathepsin G. This pathway for processing inactive pro-IL-1β is inflammasome-independent. There are at least five cleavage sites in the IL-1β pro-protein, distributed along amino acids 1-219. These processing enzymes yield different active products all containing a minimal active domain that span amino acids 120-266. The caspase 1 pathway is the most efficient pathway and yields the strongest IL-1β activity (Netea et al. 2010; Afofina et al. 2015; Lopez-Catej on et al. 2011 Cytokine and Growth Factor Reviews 22(4):189-195). Active IL-1β is secreted into the extracellular environment via mechanism that are still not well understood. Unlike other proteins, IL-1β is not secreted through the conventional endoplasmic reticulum-golgi apparatus system, but via lysosomal vesicles, exosomes/microvesicles or macrophage death via pyroptosis. It is observed that levels of active IL-1β are released in continuum and greatly affected by the magnitude of the infection or injury (strength of stimuli; Lopez-Castejon et al. 2011). Thus, measuring levels of active IL-1β can be correlated with the severity of inflammation and the disease phenotype. In tumor environments, chronic inflammation and increased levels of pro-inflammatory molecules promote tumor development and metastasis (Lyke et al. 2004 Infection and Immunity 72(10):5630-5637; Bent et al. 2018 Int J Mol Sci 19:2155, doi:103390/ijms19082155).

In some embodiments, the subjects that are likely to benefit from treatment with NELL1 can be selected by determining the levels in these subjects of IL-1β and other cytokines and pro-inflammatory factors known to be downregulated by the NELL1 pathway (e.g. IL-8, NF-κB, MMP1 etc.). Thus, in some embodiments, the methods of treatment (and administering a NELL1 polypeptide or nucleic acid molecule encoding the same) are preceded by a step of measuring levels of certain cytokines and pro-inflammatory factors (e.g., IL-1β, IL-8, NPκB, MMP1) levels in a subject or screening for genetic markers or genetic polymorphisms (e.g., single nucleotide polymorphisms or SNPs and other mutations) that predispose an individual to heightened or severe levels of certain cytokines such as IL-1β (e.g., IL-1B-511C/T, IL1-B-31T/C, or IL-1B+3954T; see Graziano et al. (2005) J Clin Oncol 23:2339-2345 and Zhang (2007) BMC Cancer 7:45). These SNPs can be found in IL-1β, IL-1β receptor, IL-8, caspase 1, proteases, or inflammasome components that drive processing of active IL-1β from the pro-protein. Heightened levels of these cytokines and pro-inflammatory factors, such as IL-1β can result in chronic inflammatory responses or activation of the inflammasome during tissue injury.

Circulating levels of IL-1β or other pro-inflammatory mediators (e.g., IL-8, NFκB, MMP1) can be measured in serum, plasma, urine, or soft tissues of patients using methods known to those skilled in the art, including but not limited to immunoassays, such as ELISA. Screening of IL-1β or other pro-inflammatory mediators (e.g., IL-8, NFκB, MMP1) can be measured at the protein level, and in some embodiments wherein IL-1β is measured, only active IL-1β (i.e., fragments of the pro-protein comprising the minimum active domain of amino acid residues 120-266) is measured. In some embodiments, subjects with muscle atrophy (or prone to develop muscle atrophy) that could benefit from treatment with a NELL1 polypeptide or nucleic acid encoding the same have increased circulating levels of IL-1β when compared to an appropriate control subject. In some embodiments, the control subject is one that does not exhibit chronic systemic inflammation or symptoms thereof or the control subject does not have a genetic predisposition for elevated IL-1β levels. In other embodiments, the control subject is an average measurement of circulating IL-1β levels from a population of individuals that do not exhibit systemic inflammation or symptoms thereof or do not have a genetic predisposition for elevated IL-1β levels.

In some embodiments, subjects with muscle atrophy (or are prone to develop muscle atrophy) that have circulating levels (e.g., serum, plasma, urine) of at least 1 ng/ml, at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml, at least 30 ng/ml, at least 35 ng/ml, at least 40 ng/ml, at least 50 ng/ml or more of IL-1β are treated with NELL1 polypeptide or a nucleic acid encoding the same. In certain embodiments, subjects with muscle atrophy (or those that are prone to develop muscle atrophy) that can benefit from treatment with a NELL1 polypeptide or nucleic acid encoding the same have circulating levels (e.g., serum, plasma, urine) of at least 20 ng/ml of IL-1β.

In some embodiments, the cytokine signature of a patient that would benefit from treatment with NELL1 polypeptide or a nucleic acid molecule encoding the same is one that has relatively high IL-1β, high IL-8, and/or low TNF-α when compared to an appropriate control subject (e.g., one not exhibiting chronic systemic inflammation or symptoms thereof or one not genetically predisposed to altered levels of these proteins).

IL-8 is a pro-inflammatory cytokine. Subjects that are expected to respond optimally to NELL1 can include those with circulating levels of IL-8 from at least 0.5 ng/mL, at least 0.6 ng/ml, at least 0.7 ng/ml, at least 0.8 ng/ml, at least 0.9 ng/ml, at least 1 ng/ml, at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml and higher levels. In certain embodiments, subjects with muscle atrophy (or those that are prone to develop muscle atrophy) that can benefit from treatment with a NELL1 polypeptide or a nucleic acid encoding the same have circulating levels (e.g., serum, plasma, urine) of about 0.5 ng/ml to about 10 ng/ml of IL-8. The presently disclosed methods can further comprise a step of measuring circulating levels of IL-8 to identify those subjects with muscle atrophy (or at risk of developing muscle atrophy) and relatively high levels of IL-8 when compared to an appropriate control that could benefit from treatment with NELL1. In some embodiments, the control subject is one that does not exhibit chronic systemic inflammation or symptoms thereof. In other embodiments, the control subject is an average measurement of circulating IL-8 levels from a population of individuals that do not exhibit systemic inflammation.

In some embodiments, patients that are expected to respond optimally to NELL1 include those with relatively high levels of IL-1β or IL-8 and/or relatively low TNF-α levels when compared to an appropriate control. In certain embodiments, subjects that can benefit from treatment with NELL1 include those with relatively high circulating levels of IL-1β and or IL-8 and less than 5 ng/ml, less than 4 ng/ml, less than 3 ng/ml, less than 2 ng/ml, or less than 1 ng/ml of TNF-α. In some embodiments, the subject has less than 5 ng/ml of TNF-α.

Target patient populations that are prone or hypersensitive to inflammatory responses that make them at risk for both skeletal and cardiac muscle atrophy include: a) pediatric patients, b) advanced stage cancer patients especially at Stages c) the elderly, d) patients under long term hospitalization due to disability or serious chronic diseases (Barsness K A et al. 2004 Pediatr Surg Int 20(4):238-42; Zhang D et al. 2007 BMC Cancer 7:45); and e) patients with microbial infections (e.g., bacterial, viral, fungal).

Data provided herein suggest pediatric subjects with muscle atrophy are especially sensitive to treatment with NELL1. Without being held to a particular theory or mechanism of action, studies show that children and adults have different responses to inflammatory cytokines (Barsness K A et al. 2004 Pediatr Surg Int 20(4):238-42). For instance, IL-1β-induced IL-6 and TNF-α production is higher in pediatric compared to adult peritoneal macrophages (PM). Pediatric PMs also showed an 11-fold increase in IL-1β-induced IL-10 levels but adult PMs did not produce IL-10. Thus, in some embodiments of the presently disclosed methods, the subject that is administered a NELL1 polypeptide or nucleic acid molecule encoding the same is a pediatric subject. A pediatric subject may refer to a younger subject who is still in a growth phase (e.g., net anabolic muscle growth) and/or with robust metabolism. In some embodiments, a pediatric subject is a subject that has yet to enter and/or is experiencing puberty. A pediatric subject may refer to a human subject who is less than 18 years of age, less than 17 years of age, less than 16 years of age, less than 15 years of age, less than 14 years of age, less than 13 years of age, less than 12 years of age, less than 11 years of age, or less than 10 years of age. In certain embodiments, the human subject is about 16, about 15, about 14, about 13, about 12, about 11, about 10, about 9, about 8, about 7, about 6, about 5, about 4, about 3, about 2, about 1 or less years of age. In some embodiments, a pediatric subject refers to a human subject that is no more than 8 years old.

In certain embodiments, the subject that is administered a NELL1 polypeptide or nucleic acid molecule encoding the same has cachexia or is at risk for developing cachexia. Cachexia is a form of muscle atrophy that is associated with extreme weight loss along with muscle wasting due to an underlying illness that cannot be entirely reversed with nutritional supplementation. Cachexia can be caused by various diseases, including cancer, congestive heart failure, chronic obstructive pulmonary disease, chronic kidney disease, AIDS and viral infections (Seelander M et al. 2015 Inflammation in cachexia. Mediators of Inflammation, Vol 2015, Article ID 536954).

IL-1β is a cytokine that is highly elevated during chronic inflammation and when this occurs within a cancer environment, it promotes muscle wasting (i.e., cancer cachexia), tumor development and metastases. (Argiles J M et al. 2006; Graziano F et al. 2005 J Clin Oncol 23(10):2339-45; Melstrom L G et al. 2007 Histol Histopath 22(7):805-14; Bent et al. 2018 Int. J. Mol. Sci. 19, 2155, doi:10.3390/ijms19082155). The impact of IL-1β pro-inflammatory pathways in muscle wasting is particularly pronounced in advanced pancreatic and gastric cancer, the two cancers most prone to cachexia (Melstrom L G et al. 2007; Graziano F et al. 2005; Zhang D et al. 2007 BMC Cancer 7:45). Cancer cachexia results in severe muscle wasting in both skeletal and cardiac muscles in lung, pancreatic and GI cancers (Barkhudaryan A et al. 2017 ESC Heart Failure 4:458-567).

In some embodiments, the NELL1 polypeptide or nucleic acid molecule encoding the same may be administered to a subject having cancer. Subjects may be selected for treatment on the basis of a cancer diagnosis or prognosis. NELL1 may be administered to a cancer subject for the treatment of cachexia and/or muscle atrophy associated with the cancer. Cancers may include both cancers of the blood and solid tumor cancers. For instance, cancers may include human cancers and carcinomas, sarcomas, adenocarcinomas, lymphomas, leukemias, melanomas, etc., including solid and lymphoid cancers, kidney, breast, lung, bladder, colon, ovarian, prostate, pancreas, stomach, brain, head and neck, skin, uterine, testicular, glioma, esophagus, liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), and/or multiple myeloma. Thus, in some instances, cancer may refer to lung cancer, breast cancer, ovarian cancer, leukemia, lymphoma, melanoma, pancreatic cancer, sarcoma, bladder cancer, bone cancer, brain cancer, cervical cancer, colon cancer, esophageal cancer, gastric cancer, liver cancer, head and neck cancer, kidney cancer, myeloma, thyroid cancer, prostate cancer, metastatic cancer, or carcinoma.

Leukemias generally refer to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Leukemias can include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblasts leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

Sarcomas generally refer to tumors which are made up of a substance like the embryonic connective tissue and is generally composed of closely packed cells embedded in a fibrillar or homogeneous substance. Sarcomas may include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

Melanomas generally refer to tumors arising from the melanocytic system of the skin and other organs. Melanomas may include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

Carcinomas generally refer to malignant new growths made up of epithelial cells tending to infiltrate the surrounding tissues and give rise to metastases. Carcinomas may include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

In some embodiments, exemplary cancers that may suitable for treatment with or treatment including NELL1 polypeptide or a nucleic acid encoding the same include pancreatic cancer or gastric cancer. In various implementations, treatment may be coordinated according to the progression or staging of a cancer. Cancer may generally be staged as stages 0-IV as is known in the art. Stage 0 generally refers to the condition of no cancer, but may include diagnosis of abnormal cells with the potential to become cancer. This may also be called carcinoma in situ. Stage I generally means the cancer is small and only in one area. This may also be called early-stage cancer. Stages II generally references a cancer which has grown larger but has not yet spread to other tissues and stage III generally refers to cancer which has grown into nearby tissues or lymph nodes. Stage IV generally means the cancer has spread to other parts of the subject's body. Stage IV cancer may be called advanced or metastatic cancer. Cancer may be diagnosed or staged by any method known in the art. In some embodiments, treatment of cancers or diseases, symptoms, conditions, or afflictions associated with or arising from cancer may be particularly suitable for late stage cancers. Late stage cancers include stage IV cancer. In some embodiments, late stage cancers may include stage III or stage IV cancer. Muscle atrophy, cachexia, or other diseases, conditions, symptoms, or afflictions related to an imbalance in muscle generation and muscle degradation may be more pronounced, advanced or severe during later stages of cancer.

Veterinary animals (e.g. cats, dogs, horses) also manifest muscle atrophy due to cancer, nutritional deficiencies and diseases. Cancer incidence is estimated at 6 million each for dogs and cats—making it a leading cause of death in companion animals especially after 10 years old (see fetchacure.org/resource-library/facts/on the world wide web). NELL1 polypeptide or a nucleic acid molecule encoding the same can also be formulated and administered to these non-human patients. Just like in humans, cancer incidence is affected by genetic background, thus susceptibility to cancer varies greatly by the breed of animals (Kent M S et al. 2018 PLoS One 13(2):e0192578). Similarly, diagnostic tools (DNA, RNA, protein, physiological metabolites and other molecular markers) assayed/detected in tissue, blood, saliva, body secretions and excretions (e.g. urine, feces) can be designed to identify which animals will benefit the most from NELL1 therapy. For example, since cancer susceptibility has been analyzed in purebred dogs, it is envisioned that genomic analysis can be applied to ascertain target dogs in mixed breeds.

In those instances wherein a nucleic acid molecule encoding a NELL1 polypeptide is administered to a subject or formulated for administration, the nucleic acid molecule can be in the form of an expression vector or viral vector (e.g., retroviral vector, adenoviral vector, adeno-associated viral vector) or can be delivered encapsulated within a liposome, nanoparticle (e.g., lipid nanoparticle), or exosome. A NELL1 polypeptide may also be delivered within a nanoparticle (e.g., lipid nanoparticle), liposome, or exosome.

The NELL1 polypeptide or nucleic acid molecule encoding the same can be administered to subjects in need thereof in the form of a composition further comprising a carrier. The term “carrier” as used herein describes a material that does not cause significant irritation to an organism and does not abrogate the biological activity and properties of the NELL1 polypeptide or nucleic acid molecule encoding the same. Carriers must be of sufficiently high purity and of sufficiently low toxicity to render them suitable for administration to a subject being treated. The carrier can be inert, or it can possess pharmaceutical benefits.

In some embodiments, the NELL1 polypeptide is administered to a subject in the form of a pharmaceutical composition. A pharmaceutical composition is a composition that is employed to prevent, reduce in intensity, cure or otherwise treat a target condition or disease that comprises an active ingredient (i.e., NELL1 polypeptide or nucleic acid molecule encoding the same) and a pharmaceutically acceptable carrier. A pharmaceutically acceptable carrier refers to one or more compatible solid or liquid filler, diluents or encapsulating substances which are suitable for administration to a human or other vertebrate animal.

Pharmaceutical compositions of the present disclosure can be formulated with suitable carriers, excipients, and other agents that provide suitable transfer, delivery, tolerance, and the like. A multitude of appropriate formulations are known to those skilled in the art. Suitable formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Pharmaceutical compositions for oral or parenteral use may be prepared into dosage forms in a unit dose suited to fit a dose of the active ingredients. Such dosage forms in a unit dose include, for example, tablets, pills, capsules, injections (ampoules), suppositories, etc.

The NELL1 polypeptide or nucleic acid molecule encoding the same may be mixed with other active materials that do not impair the desired action, or with materials that supplement the desired action. In certain embodiments, the NELL1 polypeptide or nucleic acid molecule encoding the same may be mixed or attached to molecules that target the active ingredient to particular tissues or increase its stability and persistence in blood, tissues, or other bodily fluids. Solutions or suspensions used for parenteral, intradermal, subcutaneous, intrathecal, or topical application may include, but are not limited to, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. Administered intravenously, particular carriers are physiological saline or phosphate buffered saline (PBS).

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. In some embodiments, the NELL1 polypeptide or nucleic acid molecule encoding the same is administered as an injectable material in buffered liquid solution, and in some of these embodiments, with protein stabilizers. The formulation may be frozen and later thawed for injection or kept stabilized under refrigeration or room temperature prior to use. The NELL1 polypeptide or nucleic acid molecule encoding the same can be formulated as a lyophilized powder to be reconstituted with liquid (e.g., buffered saline solution).

The NELL1 polypeptide or nucleic acid molecule encoding the same can also be administered orally as pills, tablets, or capsules, and in some of these embodiments, the pills, tablets, or capsules can have different release properties.

Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

The NELL1 polypeptide or nucleic acid molecule encoding the same can also be directly linked with molecules that allow slow release and/or increase protein stability or persistence (i.e., half-life) in the circulatory system.

Injectable depot forms can be made by forming microencapsulated matrices of the drug in biodegradable polymers such as polylactide-polyglycolide. Depending upon the ratio of drug to polymer and the nature of the particular polymer employed, the rate of drug release may be controlled. Such long acting formulations may be formulated with suitable polymeric or hydrophobic materials (for example as an emulsion in an acceptable oil) or ion exchange resins, or as sparingly soluble derivatives, for example, as a sparingly soluble salt. Examples of other biodegradable polymers include poly(orthoesters) and poly(anhydrides). Depot injectable formulations can also be prepared by entrapping the drug in liposomes or microemulsions which are compatible with body tissues.

The locally injectable formulations may be sterilized, for example, by filtration through a bacterial-retaining filter or by incorporating sterilizing agents in the form of sterile solid compositions that may be dissolved or dispersed in sterile water or other sterile injectable medium just prior to use. Injectable preparations, for example, sterile injectable aqueous or oleaginous suspensions, may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose, any bland fixed oil may be employed including synthetic mono- or diglycerides. In addition, fatty acids such as oleic acid are used in the preparation of injectables.

Formulations for parenteral (including but not limited to, subcutaneous, intradermal, intramuscular, intravenous, intraperitoneal, intrathecal intra-arterial, and intraarticular) administration include aqueous and non-aqueous sterile injection solutions that may contain antioxidants, buffers, bacteriostats and solutes, which render the formulation isotonic with the blood of the intended recipient; and aqueous and non-aqueous sterile suspensions, which may include suspending agents and thickening agents. The formulations may be presented in unit-dose or multi-dose containers, for example sealed ampules and vials, and may be stored in a freeze-dried (lyophilized) condition requiring the addition of the sterile liquid carrier, for example, saline, water-for-injection, a semi-liquid foam, or gel, immediately prior to use. Extemporaneous injection solutions and suspensions may be prepared from sterile powders, granules and tablets of the kind previously described. Alternatively, a NELL1 peptide or nucleic acid encoding the same is dissolved in a buffered liquid solution that is frozen in a unit-dose or multi-dose container and later thawed for injection or kept/stabilized under refrigeration until use.

The therapeutic agent(s) may be contained in controlled release systems. In order to prolong the effect of a drug, it often is desirable to slow the absorption of the drug from subcutaneous, intrathecal, or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. In some embodiments, the use of a long-term sustained release implant may be particularly suitable for treatment of chronic conditions. Long-term sustained release implants are well-known to those of ordinary skill in the art.

In some embodiments wherein the pharmaceutical composition is in the form of an implant, the NELL1 polypeptide or a nucleic acid molecule encoding the same is impregnated into drug eluting devices, scaffolds or matrices that are implanted or inserted via catheter into an area of muscle atrophy to deliver NELL1 in a controlled release fashion. The protein can also be linked to sutures. In those instances wherein the NELL1 peptide is delivered by genetically modified donor cells, the cells can be incorporated into a matrix containing an appropriate microenvironment to maintain, for a given time, the viability and growth of the genetically modified donor cells.

Non-limiting examples of suitable matrices include, but are not limited to, wound dressings, collagen matrix, patches, and hydrogels. The matrix can be applied to an atrophied muscle that has been exposed post-surgically, for example. In some embodiments, a rapidly degradable (e.g., 3-5 days or 1-2 weeks) scaffold or dressing is used to deliver NELL1 (e.g., calcium alginate). Rapidly degradable scaffolds or dressings allow for the release of a burst of NELL1 in the first phase of healing and activates tissue regeneration. In certain embodiments, the scaffold or dressing is simpler (e.g., consisting essentially of collagen type A), rather than a complex biological carrier, such as those made from urinary bladder or intestinal linings that may comprise various growth factors and collagens. In some embodiments, the wound dressing or matrix used to deliver NELL1 comprises or consists essentially of calcium alginate.

The NELL1 polypeptide or nucleic acid molecule encoding the same can be administered to a subject by dispensing, supplying, applying, or giving the NELL1 polypeptide or nucleic acid molecule encoding the same to the subject. Administration may be in vivo or administration directly to tissue ex vivo. Generally, NELL1 peptides, nucleic acid molecules encoding the same, or compositions comprising the NELL1 peptide or nucleic acid may be administered systemically either orally, buccally, parenterally, topically, by inhalation or insufflation (i.e., through the mouth or through the nose), or rectally in dosage unit formulations, optionally containing the conventional nontoxic pharmaceutically acceptable carriers, adjuvants, and vehicles as desired, or may be locally administered by means such as, but not limited to, injection, implantation, grafting, or topical application. Additional administration may be performed, for example, intravenously, transmucosally, transdermally, intramuscularly, subcutaneously, intraperitoneally, intrathecally, intralymphatically, intra-arterially, intralesionally, or epidurally.

Any suitable route of administration may be used to deliver the NELL1 polypeptide or nucleic acid molecule encoding the same for the purposes of muscle atrophy prevention and recovery. In certain embodiments, the NELL1 peptide or nucleic acid encoding the same is administered locally to the site of muscle atrophy. In some of these embodiments, the NELL1 polypeptide, NELL1 nucleic acid molecule, or a composition comprising the NELL1 polypeptide or NELL1 nucleic acid molecule are administered parenterally. The term “parenteral” as used herein refers to introduction into the body by way of an injection (i.e., administration by injection), including, for example, subcutaneously (i.e., an injection beneath the skin beneath the dermis into the subcutaneous tissue or “superficial fascia”), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques. A parenterally administered composition is delivered using a needle, e.g., a surgical needle. Injectable preparations, such as sterile injectable aqueous or oleaginous suspensions, may be formulated according to the known art using suitable dispersing or wetting agents and suspending agents. According to some such embodiments, the NELL1 polypeptide or nucleic acid molecule encoding the same is administered by injection.

In certain embodiments, the NELL1 polypeptide or nucleic acid molecule is administered as a spray onto a tissue, such as a muscle that has been exposed surgically. The NELL1 peptide or nucleic acid molecule can also be administered via adhesion to novel materials such as nanoparticles. Lyophilized NELL1 protein, which may or not be reconstituted as a liquid or a gel, can be placed directly onto an atrophic muscle.

Administering can be performed, for example, once, a plurality of times, and/or over one or more extended periods. Generally, an effective dose of the NELL1 peptide or nucleic acid encoding the same is administered to a subject one or more times. In certain preferred embodiments, the course of treatment will comprise multiple doses of the NELL1 peptide or nucleic acid encoding the same over a period of weeks or months. More specifically, the NELL1 peptide or nucleic acid encoding the same may be administered once every day, every two days, every three days, every four days, every five days, every six days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

An effective amount of a pharmaceutical composition of the invention is any amount that is effective to achieve its purpose (e.g., recovery from, including partial recovery, or prevention or slowing of muscle atrophy). The effective amount, usually expressed in mg/kg can be determined by routine methods during pre-clinical and clinical trials by those of skill in the art. The effective amount refers to a dose of the NELL1 polypeptide or nucleic acid molecule encoding the same that results in a detectable and sufficient increase in one or more of quantifiable muscle characteristics, such as muscle mass, fiber size, cross-sectional area, strength, power, or other functional measurement. In some embodiments, total body weight may be used to quantify the results of treatment. In some embodiments, the muscle mass or muscle characteristics of one or more particular muscles may be used to quantify the results of treatment (e.g., tibialis anterior muscle mass, gastrocnemius muscle mass, quadriceps muscle mass, biceps trachii muscle mass, triceps trachii muscle mass, deltoid muscle mass, etc.). An effective amount can be the amount sufficient to treat diseases, conditions, symptoms, and/or afflictions which can be cured, alleviated, managed or improved by increasing muscle mass, strength, power and/or function. For instance, in some embodiments, an effective amount will include at least about 10%, 15%, 20%, 25%, 30%, 40%, 50%, 60% or more improvement relative to the same measure in the subject prior to the treatment, relative to a predicted prognosis without treatment, or relative to a control subject who did not receive treatment. An effective amount with respect to the NELL1 peptide or nucleic acid encoding the same can mean the amount of peptide (or nucleic acid) alone, or in combination with other therapies, that provides a therapeutic benefit in the treatment or management of muscle atrophy or a related disease or condition, which can include a decrease in severity of disease symptoms, an increase in frequency and duration of disease symptom-free periods, or a prevention of impairment or disability due to the disease affliction. The term can encompass an amount that improves overall therapy, reduces or avoids unwanted effects, or enhances the therapeutic efficacy of or synergies with another therapeutic agent. In some embodiments, an effective amount of NELL1 peptide may comprise a dose administered between about 0.0001-100 mg/kg of the subject body weight (e.g., 0.0001 mg/kg, 0.0005 mg/kg, 0.001 mg/kg, 0.005 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.10 mg/kg, 0.20 mg/kg, 0.30 mg/kg, 0.40 mg/kg, 0.5 mg/kg, 0.6 mg/kg, 0.7 mg/kg, 0.8 mg/kg, 0.9 mg/kg, 1.0 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, etc.).

Treatments disclosed herein may be administered to a subject in a single dose or as multiple doses over a period of time. For instance, the treatments may be administered over a defined time course according to a treatment regimen. Doses of treatment may be administered sequentially, meaning each of the doses is administered to the subject at a different point in time, e.g., separated by a predetermined interval of hours, days, weeks, or months. For example, in some embodiments, a subsequent dose may be administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the immediately preceding dose. In some embodiments, two or more doses (e.g., all of the doses) may comprise the same amount of active ingredient (i.e. NELL1 polypeptide or nucleic acid molecule encoding the same). In some embodiments, the amount of each dose may be modulated (increased or decreased) over time according to a predetermined regimen and/or according to the subject's response to treatment. For instance, the dosage may be increased in subjects who do not display sufficiently improved measurements or outcomes or, alternatively, the dosage may be decreased in subjects who display adverse side effects.

The NELL1 polypeptide or nucleic acid molecule encoding the same can be administered prior to, along with, or subsequent to another treatment for recovery from or prevention of muscle atrophy, including one or more additional therapeutic agents (i.e. active ingredients). In some embodiments, additional treatments may be configured for treating muscle atrophy and/or related diseases, conditions, symptoms, or afflictions as described elsewhere herein. In some embodiments, additional treatments may be configured for treating cancer (e.g., pancreatic or gastric cancer). Non-limiting examples of other treatments include surgery, rehabilitation, cryotherapy, administration of precursor cells, extracellular matrix materials (synthetic or purified), anti-inflammatory agents/immunosuppressants, analgesics, growth factor inhibitors, metabolic inhibitors, enzyme inhibitors, and/or cytotoxic/cytostatic agents. Combination therapy generally refers to co-administration of two or more biologically active agents (e.g., drugs) used in conjunction with each other. Combination therapy may comprise a single formulation or multiple formulations. In some embodiments, combination therapies may include 2, 3, 4, 5, or more individual therapies. Co-administration may be carried out as concurrent administration or serial administration. Co-administration may be carried out via the same route of administration or different routes of administration. In some embodiments, combination therapeutic agents may be administered via the same carrier (e.g., a pharmaceutically acceptable carrier). In some embodiments, combination therapeutic agents may be administered via separate carriers or vehicles, whether administered substantially simultaneously or sequentially. Combination therapy may include two or more therapies in which the effects overlap in the subject for purposes of achieving supplemental, additive or synergistic clinical effects. In some implementations, the dosage, the effective amount, and/or the administration regimen of an individual therapeutic agent (e.g., the NELL1 polypeptide) may be adjusted relative to the dosage, the effective amount, and/or the administration regimen of the therapeutic agent when delivered alone (i.e. not as part of a combination therapy). For instance, the dosage, the effective amount, and/or the frequency of administration may be reduced. In other embodiments, the dosage, the effective amount, and/or the administration regimen may remain substantially the same.

NELL1 can be combined with cells that are important in the formation of muscle tissue. Cells may be naturally extracted from the subject, an allograft, or a xenograft or may be synthetically engineered. Cells may be expanded, treated, and/or genetically modified in vitro prior to administration to the subject. For example, a NELL1 polypeptide or nucleic acid molecule encoding the same can be formulated or delivered in combination (simultaneously or sequentially) with other biomolecules and/or adult stem cells, (naturally extracted and expanded or engineered; autologous, allogeneic, or xenogeneic), such as mesenchymal stem cells, to create complex regenerative mixtures or cocktails that are injected, implanted or infused for systemic release into a subject. Treatments may comprise the administration of complex regenerative mixtures or cocktails that can be injected, implanted, infused or otherwise administered to the subject. The administration of the mixture or cocktail may induce systemic release of the NELL1 peptide or nucleic acid encoding the same into the subject or may deliver NELL1 to a local region (e.g., local cells, local muscle, local tissue, or local region or body part).

NELL1 can be added to formulations or (or used along with) products that are acellular extracellular matrix materials either extracted from natural sources (e.g. linings of urinary bladder, small intestinal submucosa, decellularized tissue from the subject, an allograft, or a xenograft, etc.) or manufactured as a synthetic. Acellular products for regenerative medicine that contain extracellular matrix material may not have all the needed signals for tissue regeneration and the addition of NELL1 can enhance the ability of some of these materials to effect cell differentiation and tissue maturation. The NELL1 polypeptide or nucleic acid molecule encoding the same may be impregnated, linked (e.g., covalently conjugated or non-covalently associated with), infused, integrated, or otherwise coupled with synthetic and/or natural matrix/scaffold materials that are administered by implantation into the body. The matrix/scaffold material may include synthetic and/or natural polymers, including but not limited to chitosan, agarose, alginate, gelatin, collagen, hyaluronic acid, fibrinogen, fibronectin, myoglobin, hemoglobin, polyethyelene glycol (PEG), polylactic acid (PLA), poly(lactic-co-glycolic acid) (PLGA), polycaprolactone, silk fibroin, ethylene vinyl acetate copolymer, etc. In some embodiments, the matrix or scaffold material may be slowly degraded to release components into the blood, thoracic or gastric cavity or muscle to promote muscle structural and physiological recovery or new muscle formation. In various implementations, one or more active ingredients may be released upon degradation/dissolution of the matrix/scaffold materials (e.g., physiological degradation such as enzymatic degradation and/or hydrolysis), upon breaking covalent linkages to the matrix/scaffold material, and/or upon diffusion form the matrix scaffold material. In some embodiments, the administered treatment may comprise both acellular matrix/scaffold material as well as cells, as described above. In various implementations, the cells may be genetically modified and/or transfected (e.g., may be modified to incorporate a vector such as a plasmid) to express nucleic acids encoding the NELL1 peptide.

In practicing combination therapy, the NELL1 polypeptide or nucleic acid molecule encoding the same and the additional treatment or therapeutic agent may be administered to the subject simultaneously, either in a single composition, or as two or more distinct compositions using the same or different administration routes. Alternatively, the NELL1 polypeptide or nucleic acid molecule encoding the same may precede, or follow, the additional treatment or therapeutic agent by, e.g., intervals ranging from minutes to weeks. In at least one embodiment, the NELL1 polypeptide or nucleic acid molecule encoding the same and the additional treatment or therapeutic agent are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the NELL1 polypeptide or nucleic acid molecule encoding the same and the additional treatment or therapeutic agent.

### III. Miscellaneous

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In addition, ranges provided in the specification and appended claims include both end points and all points between the end points. Therefore, a range of 2.0 to 3.0 includes 2.0, 3.0, and all points between 2.0 and 3.0.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

General methods in molecular genetics and genetic engineering useful in the present invention are described in the current editions of Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). Reagents, cloning vectors, and kits for genetic manipulation are available from commercial vendors such as BioRad, Stratagene, Invitrogen, ClonTech and Sigma-Aldrich Co.

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding. No unnecessary limitations are to be understood therefrom. The invention is not limited to the exact details shown and described. Variations obvious to one skilled in the art are included in the invention defined by the claims. Any section headings used herein are for organizational purposes and are not to be construed as limiting the subject matter described.

### IV. Sequence Summary and Sequences

The following Table 1 provides a summary of the included sequences.

TABLE 1
Nucleotide and amino acid sequences disclosed herein.

| SEQ ID NO. | Description |
|---|---|
| 1 | Homo sapiens NELL1 isoform 1 transcript variant |
| | (nucleotide) |
| 2 | Homo sapiens NELL1 isoform 1 (amino acid) |
| 3 | Homo sapiens NELL1 isoform 2 transcript variant |
| | (nucleotide) |
| 4 | Homo sapiens NELL1 isoform 2 (amino acid) |
| 5 | Equus caballus NELL1 isoform 1 (nucleotide) |
| 6 | Equus caballus NELL1 isoform 1 (amino acid) |
| 7 | Equus caballus NELL1 isoform 2 (nucleotide) |
| 8 | Equus caballus NELL1 isoform 2 (amino acid) |
| 9 | Mus musculus NELL1 (nucleotide) |
| 10 | Mus musculus NELL1 (amino acid) |
| 11 | Rattus norvegicus NELL1 (nucleotide) |
| 12 | Rattus norvegicus NELL1 (amino acid) |
| 13 | Felis catus NELL1 isoform 1 (amino acid) |
| 14 | Felis catus NELL1 isoform 2 (amino acid) |
| 15 | Canis lupis familiaris NELL1 (amino acid) |
| 16 | Ovis aries NELL1 (amino acid) |
| 17 | Homo sapiens NELL1 fragment (amino acid) |
| 18 | Equus caballus NELL1 fragment (amino acid) |
| 19 | Bos taurus NELL1 (amino acid) |
| 20 | Homo sapiens interleukin-1 beta (amino acid) |

Homo sapiens NELL1 isoform 1 nucleotide sequence (SEQ ID NO: 1) and
translated amino acid sequence (SEQ ID NO: 2)

| | |
|---|---|
| atatgcgagc gcagcacccg gcgctgccga gccacctccc ccgccgcccg ctagcaagtt | 60 |
| tggcggctcc aagccaggcg cgcctcagga tccaggctca tttgcttcca cctagcttcg | 120 |
| gtgccccctg ctaggcgggg accctcgaga gcg atg ccg atg gat ttg att tta | 174 |
| Met Pro Met Asp Leu Ile Leu | |
| gtt gtg tgg ttc tgt gtg tgc act gcc agg aca gtg gtg ggc ttt ggg | 222 |
| Val Val Trp Phe Cys Val Cys Thr Ala Arg Thr Val Val Gly Phe Gly | |
| atg gac cct gac ctt cag atg gat atc gtc acc gag ctt gac ctt gtg | 270 |
| Met Asp Pro Asp Leu Gln Met Asp Ile Val Thr Glu Leu Asp Leu Val | |
| aac acc acc ctt gga gtt gct cag gtg tct gga atg cac aat gcc agc | 318 |
| Asn Thr Thr Leu Gly Val Ala Gln Val Ser Gly Met His Asn Ala Ser | |
| aaa gca ttt tta ttt caa gac ata gaa aga gag atc cat gca gct cct | 366 |
| Lys Ala Phe Leu Phe Gln Asp Ile Glu Arg Glu Ile His Ala Ala Pro | |
| cat gtg agt gag aaa tta att cag ctg ttc cgg aac aag agt gaa ttc | 414 |
| His Val Ser Glu Lys Leu Ile Gln Leu Phe Arg Asn Lys Ser Glu Phe | |
| acc att ttg gcc act gta cag cag aag cca tcc act tca gga gtg ata | 462 |
| Thr Ile Leu Ala Thr Val Gln Gln Lys Pro Ser Thr Ser Gly Val Ile | |
| ctg tcc att cga gaa ctg gag cac agc tat ttt gaa ctg gag agc agt | 510 |
| Leu Ser Ile Arg Glu Leu Glu His Ser Tyr Phe Glu Leu Glu Ser Ser | |
| ggc ctg agg gat gag att cgg tat cac tac ata cac aat ggg aag cca | 558 |
| Gly Leu Arg Asp Glu Ile Arg Tyr His Tyr Ile His Asn Gly Lys Pro | |
| agg aca gag gca ctt cct tac cgc atg gca gat gga caa tgg cac aag | 606 |
| Arg Thr Glu Ala Leu Pro Tyr Arg Met Ala Asp Gly Gln Trp His Lys | |
| gtt gca ctg tca gtt agc gcc tct cat ctc ctg ctc cat gtc gac tgt | 654 |
| Val Ala Leu Ser Val Ser Ala Ser His Leu Leu Leu His Val Asp Cys | |
| aac agg att tat gag cgt gtg ata gac cct cca gat acc aac ctt ccc | 702 |
| Asn Arg Ile Tyr Glu Arg Val Ile Asp Pro Pro Asp Thr Asn Leu Pro | |
| cca gga atc aat tta tgg ctt ggc cag cgc aac caa aag cat ggc tta | 750 |
| Pro Gly Ile Asn Leu Trp Leu Gly Gln Arg Asn Gln Lys His Gly Leu | |
| ttc aaa ggg atc atc caa gat ggg aag atc atc ttt atg ccg aat gga | 798 |
| Phe Lys Gly Ile Ile Gln Asp Gly Lys Ile Ile Phe Met Pro Asn Gly | |
| tat ata aca cag tgt cca aat cta aat cac act tgc cca acc tgc agt | 846 |
| Tyr Ile Thr Gln Cys Pro Asn Leu Asn His Thr Cys Pro Thr Cys Ser | |
| gat ttc tta agc ctg gtg caa gga ata atg gat tta caa gag ctt ttg | 894 |
| Asp Phe Leu Ser Leu Val Gln Gly Ile Met Asp Leu Gln Glu Leu Leu | |
| gcc aag atg act gca aaa cta aat tat gca gag aca aga ctt agt caa | 942 |
| Ala Lys Met Thr Ala Lys Leu Asn Tyr Ala Glu Thr Arg Leu Ser Gln | |
| ttg gaa aac tgt cat tgt gag aag act tgt caa gtg agt gga ctg ctc | 990 |
| Leu Glu Asn Cys His Cys Glu Lys Thr Cys Gln Val Ser Gly Leu Leu | |
| tat cga gat caa gac tct tgg gta gat ggt gac cat tgc agg aac tgc | 1038 |
| Tyr Arg Asp Gln Asp Ser Trp Val Asp Gly Asp His Cys Arg Asn Cys | |
| act tgc aaa agt ggt gcc gtg gaa tgc cga agg atg tcc tgt ccc cct | 1086 |
| Thr Cys Lys Ser Gly Ala Val Glu Cys Arg Arg Met Ser Cys Pro Pro | |
| ctc aat tgc tcc cca gac tcc ctc cca gtg cac att gct ggc cag tgc | 1134 |
| Leu Asn Cys Ser Pro Asp Ser Leu Pro Val His Ile Ala Gly Gln Cys | |
| tgt aag gtc tgc cga cca aaa tgt atc tat gga gga aaa gtt ctt gca | 1182 |
| Cys Lys Val Cys Arg Pro Lys Cys Ile Tyr Gly Gly Lys Val Leu Ala | |
| gaa ggc cag cgg att tta acc aag agc tgt cgg gaa tgc cga ggt gga | 1230 |
| Glu Gly Gln Arg Ile Leu Thr Lys Ser Cys Arg Glu Cys Arg Gly Gly | |
| gtt tta gta aaa att aca gaa atg tgt cct cct ttg aac tgc tca gaa | 1278 |
| Val Leu Val Lys Ile Thr Glu Met Cys Pro Pro Leu Asn Cys Ser Glu | |
| aag gat cac att ctt cct gag aat cag tgc tgc cgt gtc tgt aga ggt | 1326 |
| Lys Asp His Ile Leu Pro Glu Asn Gln Cys Cys Arg Val Cys Arg Gly | |
| cat aac ttt tgt gca gaa gga cct aaa tgt ggt gaa aac tca gag tgc | 1374 |
| His Asn Phe Cys Ala Glu Gly Pro Lys Cys Gly Glu Asn Ser Glu Cys | |
| aaa aac tgg aat aca aaa gct act tgt gag tgc aag agt ggt tac atc | 1422 |
| Lys Asn Trp Asn Thr Lys Ala Thr Cys Glu Cys Lys Ser Gly Tyr Ile | |
| tct gtc cag gga gac tct gcc tac tgt gaa gat att gat gag tgt gca | 1470 |
| Ser Val Gln Gly Asp Ser Ala Tyr Cys Glu Asp Ile Asp Glu Cys Ala | |
| gct aag atg cat tac tgt cat gcc aat act gtg tgt gtc aac ctt cct | 1518 |
| Ala Lys Met His Tyr Cys His Ala Asn Thr Val Cys Val Asn Leu Pro | |
| ggg tta tat cgc tgt gac tgt gtc cca gga tac att cgt gtg gat gac | 1566 |
| Gly Leu Tyr Arg Cys Asp Cys Val Pro Gly Tyr Ile Arg Val Asp Asp | |
| ttc tct tgt aca gaa cac gat gaa tgt ggc agc ggc cag cac aac tgt | 1614 |
| Phe Ser Cys Thr Glu His Asp Glu Cys Gly Ser Gly Gln His Asn Cys | |
| gat gag aat gcc atc tgc acc aac act gtc cag gga cac agc tgc acc | 1662 |
| Asp Glu Asn Ala Ile Cys Thr Asn Thr Val Gln Gly His Ser Cys Thr | |
| tgc aaa ccg ggc tac gtg ggg aac ggg acc atc tgc aga gct ttc tgt | 1710 |
| Cys Lys Pro Gly Tyr Val Gly Asn Gly Thr Ile Cys Arg Ala Phe Cys | |
| gaa gag ggc tgc aga tac ggt gga acg tgt gtg gct ccc aac aaa tgt | 1758 |
| Glu Glu Gly Cys Arg Tyr Gly Gly Thr Cys Val Ala Pro Asn Lys Cys | |
| gtc tgt cca tct gga ttc aca gga agc cac tgc gag aaa gat att gat | 1806 |
| Val Cys Pro Ser Gly Phe Thr Gly Ser His Cys Glu Lys Asp Ile Asp | |
| gaa tgt tca gag gga atc att gag tgc cac aac cat tcc cgc tgc gtt | 1854 |
| Glu Cys Ser Glu Gly Ile Ile Glu Cys His Asn His Ser Arg Cys Val | |
| aac ctg cca ggg tgg tac cac tgt gag tgc aga agc ggt ttc cat gac | 1902 |
| Asn Leu Pro Gly Trp Tyr His Cys Glu Cys Arg Ser Gly Phe His Asp | |
| gat ggg acc tat tca ctg tcc ggg gag tcc tgt att gac att gat gaa | 1950 |
| Asp Gly Thr Tyr Ser Leu Ser Gly Glu Ser Cys Ile Asp Ile Asp Glu | |
| tgt gcc tta aga act cac acc tgt tgg aac gat tct gcc tgc atc aac | 1998 |
| Cys Ala Leu Arg Thr His Thr Cys Trp Asn Asp Ser Ala Cys Ile Asn | |
| ctg gca ggg ggc ttt gac tgt ctc tgc ccc tct ggg ccc tcc tgc tct | 2046 |
| Leu Ala Gly Gly Phe Asp Cys Leu Cys Pro Ser Gly Pro Ser Cys Ser | |
| ggt gac tgt cct cat gaa ggg ggg ctg aag cac aat ggc cag gtg tgg | 2094 |
| Gly Asp Cys Pro His Glu Gly Gly Leu Lys His Asn Gly Gln Val Trp | |
| acc ttg aaa gaa gac agg tgt tct gtc tgc tcc tgc aag gat ggc aag | 2142 |
| Thr Leu Lys Glu Asp Arg Cys Ser Val Cys Ser Cys Lys Asp Gly Lys | |
| ata ttc tgc cga cgg aca gct tgt gat tgc cag aat cca agt gct gac | 2190 |
| Ile Phe Cys Arg Arg Thr Ala Cys Asp Cys Gln Asn Pro Ser Ala Asp | |
| cta ttc tgt tgc cca gaa tgt gac acc aga gtc aca agt caa tgt tta | 2238 |
| Leu Phe Cys Cys Pro Glu Cys Asp Thr Arg Val Thr Ser Gln Cys Leu | |
| gac caa aat ggt cac aag ctg tat cga agt gga gac aat tgg acc cat | 2286 |
| Asp Gln Asn Gly His Lys Leu Tyr Arg Ser Gly Asp Asn Trp Thr His | |
| agc tgt cag cag tgt cgg tgt ctg gaa gga gag gta gat tgc tgg cca | 2334 |
| Ser Cys Gln Gln Cys Arg Cys Leu Glu Gly Glu Val Asp Cys Trp Pro | |
| ctc act tgc ccc aac ttg agc tgt gag tat aca gct atc tta gaa ggg | 2382 |
| Leu Thr Cys Pro Asn Leu Ser Cys Glu Tyr Thr Ala Ile Leu Glu Gly | |
| gaa tgt tgt ccc cgc tgt gtc agt gac ccc tgc cta gct gat aac atc | 2430 |
| Glu Cys Cys Pro Arg Cys Val Ser Asp Pro Cys Leu Ala Asp Asn Ile | |
| acc tat gac atc aga aaa act tgc ctg gac agc tat ggt gtt tca cgg | 2478 |
| Thr Tyr Asp Ile Arg Lys Thr Cys Leu Asp Ser Tyr Gly Val Ser Arg | |
| ctt agt ggc tca gtg tgg acg atg gct gga tct ccc tgc aca acc tgt | 2526 |
| Leu Ser Gly Ser Val Trp Thr Met Ala Gly Ser Pro Cys Thr Thr Cys | |
| aaa tgc aag aat gga aga gtc tgt tgt tct gtg gat ttt gag tgt ctt | 2574 |
| Lys Cys Lys Asn Gly Arg Val Cys Cys Ser Val Asp Phe Glu Cys Leu | |
| caa aat aat tga agtatttaca gtggactcaa cgcagaagaa tggacgaaat | 2626 |
| Gln Asn Asn * | |
| gaccatccaa cgtgattaag gataggaatc ggtagtttgg tttttttgtt tgttttgttt | 2686 |
| ttttaaccac agataattgc caaagtttcc acctgaggac ggtgtttgga ggttgccttt | 2746 |
| tggacctacc actttgctca ttcttgctaa cctagtctag gtgacctaca gtgccgtgca | 2806 |
| tttaagtcaa tggttgttaa aagaagtttc ccgtgttgta aatcatgttt cccttatcag | 2866 |
| atcatttgca aatacattta aatgatctca tggtaaatgt tgatgtattt tttggtttat | 2926 |
| tttgtgtact aacataatag agagagactc agctcctttt atttattttg ttgatttatg | 2986 |
| gatcaaattc taaaataaag ttgcctgttg tgacttttgt cccatctact gcatacttag | 3046 |
| tgctgagatc cctgtaaaat gttttgatga aaatatgtat gtagagtcca gtcgcattat | 3106 |
| acatacattt catagtgctg aaccttctta aatgcctact cattcagctt aaacaggctg | 3166 |
| aagccaagta tgacaaagag gggaagggcc aaaaacataa tcaaagaata attttaaaga | 3226 |
| gaattcttgt ctctcttgca aaaaaaaaa | 3255 |

Homo sapiens NELL1 isoform 1 amino acid sequence (SEQ ID NO: 2)
Met Pro Met Asp Leu Ile Leu Val Val Trp Phe Cys Val Cys Thr Ala
Arg Thr Val Val Gly Phe Gly Met Asp Pro Asp Leu Gln Met Asp Ile
Val Thr Glu Leu Asp Leu Val Asn Thr Thr Leu Gly Val Ala Gln Val
Ser Gly Met His Asn Ala Ser Lys Ala Phe Leu Phe Gln Asp Ile Glu
Arg Glu Ile His Ala Ala Pro His Val Ser Glu Lys Leu Ile Gln Leu
Phe Arg Asn Lys Ser Glu Phe Thr Ile Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Val Ser Ala Ser His
Leu Leu Leu His Val Asp Cys Asn Arg Ile Tyr Glu Arg Val Ile Asp
Pro Pro Asp Thr Asn Leu Pro Pro Gly Ile Asn Leu Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
His Thr Cys Pro Thr Cys Ser Asp Phe Leu Ser Leu Val Gln Gly Ile
Met Asp Leu Gln Glu Leu Leu ala Lys Met Thr Ala Lys Leu Asn Tyr
Ala Glu Thr Arg Leu Ser Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Glu Met Cys
Pro Pro Leu Asn Cys Ser Glu Lys Asp His Ile Leu Pro Glu Asn Gln
Cys Cys Arg Val Cys Arg Gly His Asn Phe Cys Ala Glu Gly Pro Lys
Cys Gly Glu Asn Ser Glu Cys Lys Asn Trp Asn Thr Lys Ala Thr Cys
Glu Cys Lys Ser Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Glu Glu Gly Cys Arg Tyr Gly Gly Thr
Cys Val Ala Pro Asn Lys Cys Val Cys Pro Ser Gly Phe Thr Gly Ser
His Cys Glu Lys Asp Ile Asp Glu Cys Ser Glu Gly Ile Ile Glu Cys
His Asn His Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Ile Asp Ile Asp Glu Cys Ala Leu Arg Thr His Thr Cys Trp
Asn Asp Ser Ala Cys Ile Asn Leu Ala Gly Gly Phe Asp Cys Leu Cys
Pro Ser Gly Pro Ser Cys Ser Gly Asp Cys Pro His Glu Gly Gly Leu
Lys His Asn Gly Gln Val Trp Thr Leu Lys Glu Asp Arg Cys Ser Val
Cys Ser Cys Lys Asp Gly Lys Ile Phe Cys Arg Arg Thr Ala Cys Asp
Cys Gln Asn Pro Ser Ala Asp Leu Phe Cys Cys Pro Glu Cys Asp Thr
Arg Val Thr Ser Gln Cys Leu Asp Gln Asn Gly His Lys Leu Tyr Arg
Ser Gly Asp Asn Trp Thr His Ser Cys Gln Gln Cys Arg Cys Leu Glu
Gly Glu Val Asp Cys Trp Pro Leu Thr Cys Pro Asn Leu Ser Cys Glu
Tyr Thr Ala Ile Leu Glu Gly Glu Cys Cys Pro Arg Cys Val Ser Asp
Pro Cys Leu Ala Asp Asn Ile Thr Tyr Asp Ile Arg Lys Thr Cys Leu
Asp Ser Tyr Gly Val Ser Arg Leu Ser Gly Ser Val Trp Thr Met Ala
Gly Ser Pro Cys Thr Thr Cys Lys Cys Lys Asn Gly Arg Val Cys Cys
Ser Val Asp Phe Glu Cys Leu Gln Asn Asn
Homo sapiens NELL1 isoform 2 nucleotide sequence (SEQ ID NO: 3) and
translated amino acid sequence (SEQ ID NO: 4)

| | |
|---|---|
| atatgcgagc gcagcacccg gcgctgccga gccacctccc ccgccgcccg ctagcaagtt | 60 |
| tggcggctcc aagccaggcg cgcctcagga tccaggctca tttgcttcca cctagcttcg | 120 |
| gtgccccctg ctaggcgggg accctcgaga gcg atg ccg atg gat ttg att tta | 174 |
| Met Pro Met Asp Leu Ile Leu | |
| gtt gtg tgg ttc tgt gtg tgc act gcc agg aca gtg gtg ggc ttt ggg | 222 |
| Val Val Trp Phe Cys Val Cys Thr Ala Arg Thr Val Val Gly Phe Gly | |
| atg gac cct gac ctt cag atg gat atc gtc acc gag ctt gac ctt gtg | 270 |
| Met Asp Pro Asp Leu Gln Met Asp Ile Val Thr Glu Leu Asp Leu Val | |
| aac acc acc ctt gga gtt gct cag gtg tct gga atg cac aat gcc agc | 318 |
| Asn Thr Thr Leu Gly Val Ala Gln Val Ser Gly Met His Asn Ala Ser | |
| aaa gca ttt tta ttt caa gac ata gaa aga gag atc cat gca gct cct | 366 |
| Lys Ala Phe Leu Phe Gln Asp Ile Glu Arg Glu Ile His Ala Ala Pro | |
| cat gtg agt gag aaa tta att cag ctg ttc cgg aac aag agt gaa ttc | 414 |
| His Val Ser Glu Lys Leu Ile Gln Leu Phe Arg Asn Lys Ser Glu Phe | |
| acc att ttg gcc act gta cag cag aag cca tcc act tca gga gtg ata | 462 |
| Thr Ile Leu Ala Thr Val Gln Gln Lys Pro Ser Thr Ser Gly Val Ile | |
| ctg tcc att cga gaa ctg gag cac agc tat ttt gaa ctg gag agc agt | 510 |
| Leu Ser Ile Arg Glu Leu Glu His Ser Tyr Phe Glu Leu Glu Ser Ser | |
| ggc ctg agg gat gag att cgg tat cac tac ata cac aat ggg aag cca | 558 |
| Gly Leu Arg Asp Glu Ile Arg Tyr His Tyr Ile His Asn Gly Lys Pro | |
| agg aca gag gca ctt cct tac cgc atg gca gat gga caa tgg cac aag | 606 |
| Arg Thr Glu Ala Leu Pro Tyr Arg Met Ala Asp Gly Gln Trp His Lys | |
| gtt gca ctg tca gtt agc gcc tct cat ctc ctg ctc cat gtc gac tgt | 654 |
| Val Ala Leu Ser Val Ser Ala Ser His Leu Leu Leu His Val Asp Cys | |
| aac agg att tat gag cgt gtg ata gac cct cca gat acc aac ctt ccc | 702 |
| Asn Arg Ile Tyr Glu Arg Val Ile Asp Pro Pro Asp Thr Asn Leu Pro | |
| cca gga atc aat tta tgg ctt ggc cag cgc aac caa aag cat ggc tta | 750 |
| Pro Gly Ile Asn Leu Trp Leu Gly Gln Arg Asn Gln Lys His Gly Leu | |
| ttc aaa ggg atc atc caa gat ggg aag atc atc ttt atg ccg aat gga | 798 |
| Phe Lys Gly Ile Ile Gln Asp Gly Lys Ile Ile Phe Met Pro Asn Gly | |
| tat ata aca cag tgt cca aat cta aat cac act tgc cca acc tgc agt | 846 |
| Tyr Ile Thr Gln Cys Pro Asn Leu Asn His Thr Cys Pro Thr Cys Ser | |
| gat ttc tta agc ctg gtg caa gga ata atg gat tta caa gag ctt ttg | 894 |
| Asp Phe Leu Ser Leu Val Gln Gly Ile Met Asp Leu Gln Glu Leu Leu | |
| gcc aag atg act gca aaa cta aat tat gca gag aca aga ctt agt caa | 942 |
| Ala Lys Met Thr Ala Lys Leu Asn Tyr Ala Glu Thr Arg Leu Ser Gln | |
| ttg gaa aac tgt cat tgt gag aag act tgt caa gtg agt gga ctg ctc | 990 |
| Leu Glu Asn Cys His Cys Glu Lys Thr Cys Gln Val Ser Gly Leu Leu | |
| tat cga gat caa gac tct tgg gta gat ggt gac cat tgc agg aac tgc | 1038 |
| Tyr Arg Asp Gln Asp Ser Trp Val Asp Gly Asp His Cys Arg Asn Cys | |
| act tgc aaa agt ggt gcc gtg gaa tgc cga agg atg tcc tgt ccc cct | 1086 |
| Thr Cys Lys Ser Gly Ala Val Glu Cys Arg Arg Met Ser Cys Pro Pro | |
| ctc aat tgc tcc cca gac tcc ctc cca gtg cac att gct ggc cag tgc | 1134 |
| Leu Asn Cys Ser Pro Asp Ser Leu Pro Val His Ile Ala Gly Gln Cys | |
| tgt aag gtc tgc cga cca aaa tgt atc tat gga gga aaa gtt ctt gca | 1182 |
| Cys Lys Val Cys Arg Pro Lys Cys Ile Tyr Gly Gly Lys Val Leu Ala | |
| gaa ggc cag cgg att tta acc aag agc tgt cgg gaa tgc cga ggt gga | 1230 |
| Glu Gly Gln Arg Ile Leu Thr Lys Ser Cys Arg Glu Cys Arg Gly Gly | |
| gtt tta gta aaa att aca gaa atg tgt cct cct ttg aac tgc tca gaa | 1278 |
| Val Leu Val Lys Ile Thr Glu Met Cys Pro Pro Leu Asn Cys Ser Glu | |
| aag gat cac att ctt cct gag aat cag tgc tgc cgt gtc tgt aga ggt | 1326 |
| Lys Asp His Ile Leu Pro Glu Asn Gln Cys Cys Arg Val Cys Arg Gly | |
| cat aac ttt tgt gca gaa gga cct aaa tgt ggt gaa aac tca gag tgc | 1374 |
| His Asn Phe Cys Ala Glu Gly Pro Lys Cys Gly Glu Asn Ser Glu Cys | |
| aaa aac tgg aat aca aaa gct act tgt gag tgc aag agt ggt tac atc | 1422 |
| Lys Asn Trp Asn Thr Lys Ala Thr Cys Glu Cys Lys Ser Gly Tyr Ile | |
| tct gtc cag gga gac tct gcc tac tgt gaa gat att gat gag tgt gca | 1470 |
| Ser Val Gln Gly Asp Ser Ala Tyr Cys Glu Asp Ile Asp Glu Cys Ala | |
| gct aag atg cat tac tgt cat gcc aat act gtg tgt gtc aac ctt cct | 1518 |
| Ala Lys Met His Tyr Cys His Ala Asn Thr Val Cys Val Asn Leu Pro | |
| ggg tta tat cgc tgt gac tgt gtc cca gga tac att cgt gtg gat gac | 1566 |
| Gly Leu Tyr Arg Cys Asp Cys Val Pro Gly Tyr Ile Arg Val Asp Asp | |
| ttc tct tgt aca gaa cac gat gaa tgt ggc agc ggc cag cac aac tgt | 1614 |
| Phe Ser Cys Thr Glu His Asp Glu Cys Gly Ser Gly Gln His Asn Cys | |
| gat gag aat gcc atc tgc acc aac act gtc cag gga cac agc tgc acc | 1662 |
| Asp Glu Asn Ala Ile Cys Thr Asn Thr Val Gln Gly His Ser Cys Thr | |
| tgc aaa ccg ggc tac gtg ggg aac ggg acc atc tgc aga gct ttc tgt | 1710 |
| Cys Lys Pro Gly Tyr Val Gly Asn Gly Thr Ile Cys Arg Ala Phe Cys | |
| gaa gag ggc tgc aga tac ggt gga acg tgt gtg gct ccc aac aaa tgt | 1758 |
| Glu Glu Gly Cys Arg Tyr Gly Gly Thr Cys Val Ala Pro Asn Lys Cys | |
| gtc tgt cca tct gga ttc aca gga agc cac tgc gag aaa gac att gat | 1806 |
| Val Cys Pro Ser Gly Phe Thr Gly Ser His Cys Glu Lys Asp Ile Asp | |
| gaa tgt gcc tta aga act cac acc tgt tgg aac gat tct gcc tgc atc | 1854 |
| Glu Cys Ala Leu Arg Thr His Thr Cys Trp Asn Asp Ser Ala Cys Ile | |
| aac ctg gca ggg ggc ttt gac tgt ctc tgc ccc tct ggg ccc tcc tgc | 1902 |
| Asn Leu Ala Gly Gly Phe Asp Cys Leu Cys Pro Ser Gly Pro Ser Cys | |
| tct ggt gac tgt cct cat gaa ggg ggg ctg aag cac aat ggc cag gtg | 1950 |
| Ser Gly Asp Cys Pro His Glu Gly Gly Leu Lys His Asn Gly Gln Val | |
| tgg acc ttg aaa gaa gac agg tgt tct gtc tgc tcc tgc aag gat ggc | 1998 |
| Trp Thr Leu Lys Glu Asp Arg Cys Ser Val Cys Ser Cys Lys Asp Gly | |
| aag ata ttc tgc cga cgg aca gct tgt gat tgc cag aat cca agt gct | 2046 |
| Lys Ile Phe Cys Arg Arg Thr Ala Cys Asp Cys Gln Asn Pro Ser Ala | |
| gac cta ttc tgt tgc cca gaa tgt gac acc aga gtc aca agt caa tgt | 2094 |
| Asp Leu Phe Cys Cys Pro Glu Cys Asp Thr Arg Val Thr Ser Gln Cys | |
| tta gac caa aat ggt cac aag ctg tat cga agt gga gac aat tgg acc | 2142 |
| Leu Asp Gln Asn Gly His Lys Leu Tyr Arg Ser Gly Asp Asn Trp Thr | |
| cat agc tgt cag cag tgt cgg tgt ctg gaa gga gag gta gat tgc tgg | 2190 |
| His Ser Cys Gln Gln Cys Arg Cys Leu Glu Gly Glu Val Asp Cys Trp | |
| cca ctc act tgc ccc aac ttg agc tgt gag tat aca gct atc tta gaa | 2238 |
| Pro Leu Thr Cys Pro Asn Leu Ser Cys Glu Tyr Thr Ala Ile Leu Glu | |
| ggg gaa tgt tgt ccc cgc tgt gtc agt gac ccc tgc cta gct gat aac | 2286 |
| Gly Glu Cys Cys Pro Arg Cys Val Ser Asp Pro Cys Leu Ala Asp Asn | |
| atc acc tat gac atc aga aaa act tgc ctg gac agc tat ggt gtt tca | 2334 |
| Ile Thr Tyr Asp Ile Arg Lys Thr Cys Leu Asp Ser Tyr Gly Val Ser | |
| cgg ctt agt ggc tca gtg tgg acg atg gct gga tct ccc tgc aca acc | 2382 |
| Arg Leu Ser Gly Ser Val Trp Thr Met Ala Gly Ser Pro Cys Thr Thr | |
| tgt aaa tgc aag aat gga aga gtc tgt tgt tct gtg gat ttt gag tgt | 2430 |
| Cys Lys Cys Lys Asn Gly Arg Val Cys Cys Ser Val Asp Phe Glu Cys | |
| ctt caa aat aat tga agtatttaca gtggactcaa cgcagaagaa tggacgaaat | 2485 |
| Leu Gln Asn Asn * | |
| gaccatccaa cgtgattaag gataggaatc ggtagtttgg tttttttgtt tgttttgttt | 2545 |
| ttttaaccac agataattgc caaagtttcc acctgaggac ggtgtttgga ggttgccttt | 2605 |
| tggacctacc actttgctca ttcttgctaa cctagtctag gtgacctaca gtgccgtgca | 2665 |
| tttaagtcaa tggttgttaa aagaagtttc ccgtgttgta aatcatgttt cccttatcag | 2725 |
| atcatttgca aatacattta aatgatctca tggtaaatgt tgatgtattt tttggtttat | 2785 |
| tttgtgtact aacataatag agagagactc agctcctttt atttattttg ttgatttatg | 2845 |
| gatcaaattc taaaataaag ttgcctgttg tgacttttgt cccatctact gcatacttag | 2905 |
| tgctgagatc cctgtaaaat gttttgatga aaatatgtat gtagagtcca gtcgcattat | 2965 |
| acatacattt catagtgctg aaccttctta aatgcctact cattcagctt aaacaggctg | 3025 |
| aagccaagta tgacaaagag gggaagggcc aaaaacataa tcaaagaata attttaaaga | 3085 |
| gaattcttgt ctctcttgca aaaaaaaaa | 3114 |

Homo sapiens NELL1 isoform 2 amino acid sequence (SEQ ID NO: 4)
Met Pro Met Asp Leu Ile Leu Val Val Trp Phe Cys Val Cys Thr Ala
Arg Thr Val Val Gly Phe Gly Met Asp Pro Asp Leu Gln Met Asp Ile
Val Thr Glu Leu Asp Leu Val Asn Thr Thr Leu Gly Val Ala Gln Val
Ser Gly Met His Asn Ala Ser Lys Ala Phe Leu Phe Gln Asp Ile Glu
Arg Glu Ile His Ala Ala Pro His Val Ser Glu Lys Leu Ile Gln Leu
Phe Arg Asn Lys Ser Glu Phe Thr Ile Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Val Ser Ala Ser His
Leu Leu Leu His Val Asp Cys Asn Arg Ile Tyr Glu Arg Val Ile Asp
Pro Pro Asp Thr Asn Leu Pro Pro Gly Ile Asn Leu Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
His Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Ser Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Glu Met Cys
Pro Pro Leu Asn Cys Ser Glu Lys Asp His Ile Leu Pro Glu Asn Gln
Cys Cys Arg Val Cys Arg Gly His Asn Phe Cys Ala Glu Gly Pro Lys
Cys Gly Glu Asn Ser Glu Cys Lys Asn Trp Asn Thr Lys Ala Thr Cys
Glu Cys Lys Ser Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Glu Glu Gly Cys Arg Tyr Gly Gly Thr
Cys Val Ala Pro Asn Lys Cys Val Cys Pro Ser Gly Phe Thr Gly Ser
His Cys Glu Lys Asp Ile Asp Glu Cys Ala Leu Arg Thr His Thr Cys
Trp Asn Asp Ser Ala Cys Ile Asn Leu Ala Gly Gly Phe Asp Cys Leu
Cys Pro Ser Gly Pro Ser Cys Ser Gly Asp Cys Pro His Glu Gly Gly
Leu Lys His Asn Gly Gln Val Trp Thr Leu Lys Glu Asp Arg Cys Ser
Val Cys Ser Cys Lys Asp Gly Lys Ile Phe Cys Arg Arg Thr Ala Cys
Asp Cys Gln Asn Pro Ser Ala Asp Leu Phe Cys Cys Pro Glu Cys Asp
Thr Arg Val Thr Ser Gln Cys Leu Asp Gln Asn Gly His Lys Leu Tyr
Arg Ser Gly Asp Asn Trp Thr His Ser Cys Gln Gln Cys Arg Cys Leu
Glu Gly Glu Val Asp Cys Trp Pro Leu Thr Cys Pro Asn Leu Ser Cys
Glu Tyr Thr Ala Ile Leu Glu Gly Glu Cys Cys Pro Arg Cys Val Ser
Asp Pro Cys Leu Ala Asp Asn Ile Thr Tyr Asp Ile Arg Lys Thr Cys
Leu Asp Ser Tyr Gly Val Ser Arg Leu Ser Gly Ser Val Trp Thr Met
Ala Gly Ser Pro Cys Thr Thr Cys Lys Cys Lys Asn Gly Arg Val Cys
Cys Ser Val Asp Phe Glu Cys Leu Gln Asn Asn
Equus caballus NELL1 isoform 1 nucleotide sequence (SEQ ID NO: 5) and
translated amino acid sequence (SEQ ID NO: 6)

| | |
|---|---|
| atg ggc ttt ggg atg gac ccc gac ctt caa atg gat att atc acc gag | 48 |
| Met Gly Phe Gly Met Asp Pro Asp Leu Gln Met Asp Ile Ile Thr Glu | |
| ctc gac ctc gtg aac acc acc ctt gga gtc act cag gtg tcc gga ctg | 96 |
| Leu Asp Leu Val Asn Thr Thr Leu Gly Val Thr Gln Val Ser Gly Leu | |
| cac aat gcc agc aaa gca ttt tta ttt caa gat gta gag aga gag atc | 144 |
| His Asn Ala Ser Lys Ala Phe Leu Phe Gln Asp Val Glu Arg Glu Ile | |
| cat gca gcc cca cac gtg agt gag aaa tta att cag ctg ttc cgg aat | 192 |
| His Ala Ala Pro His Val Ser Glu Lys Leu Ile Gln Leu Phe Arg Asn | |
| aag agt gaa ttc acc ttt ttg gcc act gtg cag cag aag ccg tca act | 240 |
| Lys Ser Glu Phe Thr Phe Leu Ala Thr Val Gln Gln Lys Pro Ser Thr | |
| tca gga gtg ata ctg tcc att cga gaa ctg gaa aac agt tat ttt gaa | 288 |
| Ser Gly Val Ile Leu Ser Ile Arg Glu Leu Glu Asn Ser Tyr Phe Glu | |
| ctg gag agc agt ggc ctg aga gat gag att cga tat cac tac aca cac | 336 |
| Leu Glu Ser Ser Gly Leu Arg Asp Glu Ile Arg Tyr His Tyr Thr His | |
| aag ggg aag ccc agg aca gag gca ctt ccc tac cgg atg gcg gac gga | 384 |
| Lys Gly Lys Pro Arg Thr Glu Ala Leu Pro Tyr Arg Met Ala Asp Gly | |
| cgg tgg cac aag gtg gcg ctg tca gtt agc gcc tct cat ctc ctg ctc | 432 |
| Arg Trp His Lys Val Ala Leu Ser Val Ser Ala Ser His Leu Leu Leu | |
| cac atc gac tgc aac agg att tat gaa cgt gtg ata gac act cct gag | 480 |
| His Ile Asp Cys Asn Arg Ile Tyr Glu Arg Val Ile Asp Thr Pro Glu | |
| acc aac ctc ccc cca gga agc aat ttg tgg ctg ggt cag cga aac caa | 528 |
| Thr Asn Leu Pro Pro Gly Ser Asn Leu Trp Leu Gly Gln Arg Asn Gln | |
| aag cac ggc tta ttc aaa gga atc atc caa gat gga aaa atc atc ttc | 576 |
| Lys His Gly Leu Phe Lys Gly Ile Ile Gln Asp Gly Lys Ile Ile Phe | |
| atg ccg aat gga tac ata aca cag tgt ccg aac ctg aat cgc act tgc | 624 |
| Met Pro Asn Gly Tyr Ile Thr Gln Cys Pro Asn Leu Asn Arg Thr Cys | |
| cca acg tgc agt gat ttc tta agc ctg gtg caa gga atc atg gat tta | 672 |
| Pro Thr Cys Ser Asp Phe Leu Ser Leu Val Gln Gly Ile Met Asp Leu | |
| caa gag ctt ctg gcc aag atg act gcg aaa cta aat tat gca gag aca | 720 |
| Gln Glu Leu Leu Ala Lys Met Thr Ala Lys Leu Asn Tyr Ala Glu Thr | |
| cga ctt agt caa ttg gaa aac tgc cac tgc gag aag acc tgt caa gtg | 768 |
| Arg Leu Ser Gln Leu Glu Asn Cys His Cys Glu Lys Thr Cys Gln Val | |
| agt gga ctg ctc tat aga gac cag gac tcc tgg gtt gat ggc gat cac | 816 |
| Ser Gly Leu Leu Tyr Arg Asp Gln Asp Ser Trp Val Asp Gly Asp His | |
| tgt agg aac tgc acg tgc aaa agc ggc gct gtg gaa tgt cgg agg atg | 864 |
| Cys Arg Asn Cys Thr Cys Lys Ser Gly Ala Val Glu Cys Arg Arg Met | |
| tct tgt ccc cct ctc aat tgc tcc cca gac tcc ctc cct gtg cac gtt | 912 |
| Ser Cys Pro Pro Leu Asn Cys Ser Pro Asp Ser Leu Pro Val His Val | |
| gcc ggc cag tgc tgt aag gtc tgc cga cca aaa tgt atc tac gga ggg | 960 |
| Ala Gly Gln Cys Cys Lys Val Cys Arg Pro Lys Cys Ile Tyr Gly Gly | |
| aaa gtc ctt gca gaa ggc cag cgg att tta acc aag agc tgt cgg gaa | 1008 |
| Lys Val Leu Ala Glu Gly Gln Arg Ile Leu Thr Lys Ser Cys Arg Glu | |
| tgc cga ggt gga gtt tta gtg aaa att aca gaa gcg tgc cct cct ttg | 1056 |
| Cys Arg Gly Gly Val Leu Val Lys Ile Thr Glu Ala Cys Pro Pro Leu | |
| aac tgc tca gac aag gat cac att ctc cca gag aat cag tgc tgc agc | 1104 |
| Asn Cys Ser Asp Lys Asp His Ile Leu Pro Glu Asn Gln Cys Cys Ser | |
| gtc tgc aga ggt cat aac ttt tgt gcg gaa gga cct aaa tgt ggt gaa | 1152 |
| Val Cys Arg Gly His Asn Phe Cys Ala Glu Gly Pro Lys Cys Gly Glu | |
| aat tca gag tgc aaa aac tgg aat aca aaa gct act tgc gag tgc aag | 1200 |
| Asn Ser Glu Cys Lys Asn Trp Asn Thr Lys Ala Thr Cys Glu Cys Lys | |
| aat ggt tat atc tct gtc cag ggg gac tcc gcc tac tgt gaa gat atc | 1248 |
| Asn Gly Tyr Ile Ser Val Gln Gly Asp Ser Ala Tyr Cys Glu Asp Ile | |
| gat gag tgt gct gct aag atg cat tac tgt cgt gcc aat act gtg tgt | 1296 |
| Asp Glu Cys Ala Ala Lys Met His Tyr Cys Arg Ala Asn Thr Val Cys | |
| gtc aac ctg cct ggg tta tat cgg tgt gac tgt gtc ccg gga tac att | 1344 |
| Val Asn Leu Pro Gly Leu Tyr Arg Cys Asp Cys Val Pro Gly Tyr Ile | |
| cgc gtg gat gat ttc tct tgt aca gaa cat gac gaa tgt ggc agc ggg | 1392 |
| Arg Val Asp Asp Phe Ser Cys Thr Glu His Asp Glu Cys Gly Ser Gly | |
| cag cac aac tgt gat gag aat gcc atc tgc acc aac act gtc cag gga | 1440 |
| Gln His Asn Cys Asp Glu Asn Ala Ile Cys Thr Asn Thr Val Gln Gly | |
| cac agc tgc acc tgc aaa ccg ggc tac gtg ggg aat ggg acc agc tgc | 1488 |
| His Ser Cys Thr Cys Lys Pro Gly Tyr Val Gly Asn Gly Thr Ser Cys | |
| aga gcg ttc tgc gaa gag ggc tgc aga tat ggc ggg aca tgc gtg gct | 1536 |
| Arg Ala Phe Cys Glu Glu Gly Cys Arg Tyr Gly Gly Thr Cys Val Ala | |
| cct aac aaa tgt gtc tgt cct tct gga ttc aca gga agc cac tgt gag | 1584 |
| Pro Asn Lys Cys Val Cys Pro Ser Gly Phe Thr Gly Ser His Cys Glu | |
| aaa gat att gat gaa tgt aca gag gga atc att gag tgc cac aac cat | 1632 |
| Lys Asp Ile Asp Glu Cys Thr Glu Gly Ile Ile Glu Cys His Asn His | |
| tcc cgc tgc gtt aac ctg cca ggg tgg tac cac tgt gag tgc aga agc | 1680 |
| Ser Arg Cys Val Asn Leu Pro Gly Trp Tyr His Cys Glu Cys Arg Ser | |
| ggt ttc cat gac gat ggg acc tat tca ctg tcc ggg gag tcc tgt att | 1728 |
| Gly Phe His Asp Asp Gly Thr Tyr Ser Leu Ser Gly Glu Ser Cys Ile | |
| gac att gat gaa tgt gcc tta aga act cac acc tgt tgg aat gat tct | 1776 |
| Asp Ile Asp Glu Cys Ala Leu Arg Thr His Thr Cys Trp Asn Asp Ser | |
| gcc tgc atc aac ttg gca ggg ggc ttc gac tgc ctg tgt ccc tca ggg | 1824 |
| Ala Cys Ile Asn Leu Ala Gly Gly Phe Asp Cys Leu Cys Pro Ser Gly | |
| cca tcc tgc tct ggt gac tgc ccc cac gaa gga gga ctg aag cgc aac | 1872 |
| Pro Ser Cys Ser Gly Asp Cys Pro His Glu Gly Gly Leu Lys Arg Asn | |
| ggg cag gtg tgg acc ctg aaa gaa gac agg tgt tct gtg tgt tcc tgc | 1920 |
| Gly Gln Val Trp Thr Leu Lys Glu Asp Arg Cys Ser Val Cys Ser Cys | |
| aag gat ggg aag ata ttc tgc cga cgg aca gct tgt gat tgc cag aat | 1968 |
| Lys Asp Gly Lys Ile Phe Cys Arg Arg Thr Ala Cys Asp Cys Gln Asn | |
| cca agc gtt gac ctt ttc tgt tgc cca gag tgt gac acc agg gtc aca | 2016 |
| Pro Ser Val Asp Leu Phe Cys Cys Pro Glu Cys Asp Thr Arg Val Thr | |
| agt caa tgt tta gac caa aat gga cac aag ctc tat cga agt gga gac | 2064 |
| Ser Gln Cys Leu Asp Gln Asn Gly His Lys Leu Tyr Arg Ser Gly Asp | |
| aat tgg act cac agc tgt cag cag tgc cgg tgt ctg gaa gga gag gta | 2112 |
| Asn Trp Thr His Ser Cys Gln Gln Cys Arg Cys Leu Glu Gly Glu Val | |
| gat tgc tgg cca ctc act tgc ccc aga ttg agc tgt gag tac aca gcc | 2160 |
| Asp Cys Trp Pro Leu Thr Cys Pro Arg Leu Ser Cys Glu Tyr Thr Ala | |
| atc ttg gaa ggg gag tgt tgt cca cgc tgt gtc agc gac ccc tgc ctg | 2208 |
| Ile Leu Glu Gly Glu Cys Cys Pro Arg Cys Val Ser Asp Pro Cys Leu | |
| gcg gat aac atc gtc tat gac atc aga gaa act tgc ctg gac agc tat | 2256 |
| Ala Asp Asn Ile Val Tyr Asp Ile Arg Glu Thr Cys Leu Asp Ser Tyr | |
| gga gtt tca agg ctt agt ggc tca gtg tgg aca ttg gct gga tct ccc | 2304 |
| Gly Val Ser Arg Leu Ser Gly Ser Val Trp Thr Leu Ala Gly Ser Pro | |
| tgc acg acc tgc aaa tgc aag aat gga agt gtc tgc tgt tct gtg gat | 2352 |
| Cys Thr Thr Cys Lys Cys Lys Asn Gly Ser Val Cys Cys Ser Val Asp | |
| ttg gag tgt ctt cat aat aat tga aggatttaaa atggactcat gatcgccaga | 2406 |
| Leu Glu Cys Leu His Asn Asn * | |
| gaaaaatgga caaatgacca tccatgatga tgaaagaaca ggagttggtg ttttttttac | 2466 |
| cacagacaat taccaaagtc tccgtctgag gaaggtgttt gcaggttgcc ttttggacct | 2526 |
| cccactctgc tcattcttgc taacctagtc taggtgacct acagtgcatt tcagtctatg | 2586 |
| gttgttaaaa gaagttttcc gtgttgtaaa tcacgtttcc cttaccaggt cattgcaaat | 2646 |
| acatttaaat gatttcatgg taaatgttga tgtatttttt gggtttattt tgtgtactaa | 2706 |
| cataatagag attcagctgc ttttatttat ttttttcttg acttttggat caaattcaac | 2766 |
| aaataaagtt gcctgttgtg atttt | 2791 |

Equus caballus NELL1 isoform 1 amino acid sequence (SEQ ID NO: 6)
Met Gly Phe Gly Met Asp Pro Asp Leu Gln Met Asp Ile Ile Thr Glu
Leu Asp Leu Val Asn Thr Thr Leu gly Val Thr Gln Val Ser Gly Leu
His Asn Ala Ser Lys Ala Phe Leu Phe Gln Asp Val Glu Arg Glu Ile
His Ala Ala Pro His Val Ser Glu Lys Leu Ile Gln Leu Phe Arg Asn
Lys Ser Glu Phe Thr Phe Leu Ala Thr Val Gln Gln Lys Pro Ser Thr
Ser Gly Val Ile Leu Ser Ile Arg Glu Leu Glu Asn Ser Tyr Phe Glu
Leu Glu Ser Ser Gly Leu Arg Asp Glu Ile Arg Tyr His Tyr Thr His
Lys Gly Lys Pro Arg Thr Glu Ala Leu Pro Tyr Arg Met Ala Asp Gly
Arg Trp His Lys Val Ala Leu Ser Val Ser Ala Ser His Leu Leu Leu
His Ile Asp Cys Asn Arg Ile Tyr Glu Arg Val Ile Asp Thr Pro Glu
Thr Asn Leu Pro Pro Gly Ser Asn Leu Trp Leu Gly Gln Arg Asn Gln
Lys His Gly Leu Phe Lys Gly Ile Ile Gln Asp Gly Lys Ile Ile Phe
Met Pro Asn Gly Tyr Ile Thr Gln Cys Pro Asn Leu Asn Arg Thr Cys
Pro Thr Cys Ser Asp Phe Leu Ser Leu Val Gln Gly Ile Met Asp Leu
Gln Glu Leu Leu Ala Lys Met Thr Ala Lys Leu Asn Tyr Ala Glu Thr
Arg Leu Ser Gln Leu Glu Asn Cys His Cys Glu Lys Thr Cys Gln Val
Ser Gly Leu Leu Tyr Arg Asp Gln Asp Ser Trp Val Asp Gly Asp His
Cys Arg Asn Cys Thr Cys Lys Ser Gly Ala Val Glu Cys Arg Arg Met
Ser Cys Pro Pro Leu Asn Cys Ser Pro Asp Ser Leu Pro Val His Val
Ala Gly Gln Cys Cys Lys Val Cys Arg Pro Lys Cys Ile Tyr Gly Gly
Lys Val Leu Ala Glu Gly Gln Arg Ile Leu Thr Lys Ser Cys Arg Glu
Cys Arg Gly Gly Val Leu Val Lys Ile Thr Glu Ala Cys Pro Pro Leu
Asn Cys Ser Asp Lys Asp His Ile Leu Pro Glu Asn Gln Cys Cys Ser
Val Cys Arg Gly His Asn Phe Cys Ala Glu Gly Pro Lys Cys Gly Glu
Asn Ser Glu Cys Lys Asn Trp Asn Thr Lys Ala Thr Cys Glu Cys Lys
Asn Gly Tyr Ile Ser Val Gln Gly Asp Ser Ala Tyr Cys Glu Asp Ile
Asp Glu Cys Ala Ala Lys Met His Tyr Cys Arg Ala Asn Thr Val Cys
Val Asn Leu Pro Gly Leu Tyr Arg Cys Asp Cys Val Pro Gly Tyr Ile
Arg Val Asp Asp Phe Ser Cys Thr Glu His Asp Glu Cys Gly Ser Gly
Gln His Asn Cys Asp Glu Asn Ala Ile Cys Thr Asn Thr Val Gln Gly
His Ser Cys Thr Cys Lys Pro Gly Tyr Val Gly Asn Gly Thr Ser Cys
Arg Ala Phe Cys Glu Glu Gly Cys Arg Tyr Gly Gly Thr Cys Val Ala
Pro Asn Lys Cys Val Cys Pro Ser Gly Phe Thr Gly Ser His Cys Glu
Lys Asp Ile Asp Glu Cys Thr Glu Gly Ile Ile Glu Cys His Asn His
Ser Arg Cys Val Asn Leu Pro Gly Trp Tyr His Cys Glu Cys Arg Ser
Gly Phe His Asp Asp Gly Thr Tyr Ser Leu Ser Gly Glu Ser Cys Ile
Asp Ile Asp Glu Cys Ala Leu Arg Thr His Thr Cys Trp Asn Asp Ser
Ala Cys Ile Asn Leu Ala Gly Gly Phe Asp Cys Leu Cys Pro Ser Gly
Pro Ser Cys Ser Gly Asp Cys Pro His Glu Gly Gly Leu Lys Arg Asn
Gly Gln Val Trp Thr Leu Lys Glu Asp Arg Cys Ser Val Cys Ser Cys
Lys Asp Gly Lys Ile Phe Cys Arg Arg Thr Ala Cys Asp Cys Gln Asn
Pro Ser Val Asp Leu Phe Cys Cys Pro Glu Cys Asp Thr Arg Val Thr
Ser Gln Cys Leu Asp Gln Asn Gly His Lys Leu Tyr Arg Ser Gly Asp
Asn Trp Thr His Ser Cys Gln Gln Cys Arg Cys Leu Glu Gly Glu Val
Asp Cys Trp Pro Leu Thr Cys Pro Arg Leu Ser Cys Glu Tyr Thr Ala
Ile Leu Glu Gly Glu Cys Cys Pro Arg Cys Val Ser Asp Pro Cys Leu
Ala Asp Asn Ile Val Tyr Asp Ile Arg Glu Thr Cys Leu Asp Ser Tyr
Gly Val Ser Arg Leu Ser Gly Ser Val Trp Thr Leu Ala Gly Ser Pro
Cys Thr Thr Cys Lys Cys Lys Asn Gly Ser Val Cys Cys Ser Val Asp
Leu Glu Cys Leu His Asn Asn
Equus caballus NELL1 isoform 2 nucleotide sequence (SEQ ID NO: 7) and
translated amino acid sequence (SEP ID NO: 8)

| | |
|---|---|
| atg ggc ttt ggg atg gac ccc gac ctt caa atg gat att atc acc gag | 48 |
| Met Gly Phe Gly Met Asp Pro Asp Leu Gln Met Asp Ile Ile Thr Glu | |
| ctc gac ctc gtg aac acc acc ctt gga gtc act cag gtg tcc gga ctg | 96 |
| Leu Asp Leu Val Asn Thr Thr Leu Gly Val Thr Gln Val Ser Gly Leu | |
| cac aat gcc agc aaa gca ttt tta ttt caa gat gta gag aga gag atc | 144 |
| His Asn Ala Ser Lys Ala Phe Leu Phe Gln Asp Val Glu Arg Glu Ile | |
| cat gca gcc cca cac gtg agt gag aaa tta att cag ctg ttc cgg aat | 192 |
| His Ala Ala Pro His Val Ser Glu Lys Leu Ile Gln Leu Phe Arg Asn | |
| aag agt gaa ttc acc ttt ttg gcc act gtg cag cag aag ccg tca act | 240 |
| Lys Ser Glu Phe Thr Phe Leu Ala Thr Val Gln Gln Lys Pro Ser Thr | |
| tca gga gtg ata ctg tcc att cga gaa ctg gaa aac agt tat ttt gaa | 288 |
| Ser Gly Val Ile Leu Ser Ile Arg Glu Leu Glu Asn Ser Tyr Phe Glu | |
| ctg gag agc agt ggc ctg aga gat gag att cga tat cac tac aca cac | 336 |
| Leu Glu Ser Ser Gly Leu Arg Asp Glu Ile Arg Tyr His Tyr Thr His | |
| aag ggg aag ccc agg aca gag gca ctt ccc tac cgg atg gcg gac gga | 384 |
| Lys Gly Lys Pro Arg Thr Glu Ala Leu Pro Tyr Arg Met Ala Asp Gly | |
| cgg tgg cac aag gtg gcg ctg tca gtt agc gcc tct cat ctc ctg ctc | 432 |
| Arg Trp His Lys Val Ala Leu Ser Val Ser Ala Ser His Leu Leu Leu | |
| cac atc gac tgc aac agg att tat gaa cgt gtg ata gac act cct gag | 480 |
| His Ile Asp Cys Asn Arg Ile Tyr Glu Arg Val Ile Asp Thr Pro Glu | |
| acc aac ctc ccc cca gga agc aat ttg tgg ctg ggt cag cga aac caa | 528 |
| Thr Asn Leu Pro Pro Gly Ser Asn Leu Trp Leu Gly Gln Arg Asn Gln | |
| aag cac ggc tta ttc aaa gga atc atc caa gat gga aaa atc atc ttc | 576 |
| Lys His Gly Leu Phe Lys Gly Ile Ile Gln Asp Gly Lys Ile Ile Phe | |
| atg ccg aat gga tac ata aca cag tgt ccg aac ctg aat cgc act tgc | 624 |
| Met Pro Asn Gly Tyr Ile Thr Gln Cys Pro Asn Leu Asn Arg Thr Cys | |
| cca acg tgc agt gat ttc tta agc ctg gtg caa gga atc atg gat tta | 672 |
| Pro Thr Cys Ser Asp Phe Leu Ser Leu Val Gln Gly Ile Met Asp Leu | |
| caa gag ctt ctg gcc aag atg act gcg aaa cta aat tat gca gag aca | 720 |
| Gln Glu Leu Leu Ala Lys Met Thr Ala Lys Leu Asn Tyr Ala Glu Thr | |
| cga ctt agt caa ttg gaa aac tgc cac tgc gag aag acc tgt caa gtg | 768 |
| Arg Leu Ser Gln Leu Glu Asn Cys His Cys Glu Lys Thr Cys Gln Val | |
| agt gga ctg ctc tat aga gac cag gac tcc tgg gtt gat ggc gat cac | 816 |
| Ser Gly Leu Leu Tyr Arg Asp Gln Asp Ser Trp Val Asp Gly Asp His | |
| tgt agg aac tgc acg tgc aaa agc ggc gct gtg gaa tgt cgg agg atg | 864 |
| Cys Arg Asn Cys Thr Cys Lys Ser Gly Ala Val Glu Cys Arg Arg Met | |
| tct tgt ccc cct ctc aat tgc tcc cca gac tcc ctc cct gtg cac gtt | 912 |
| Ser Cys Pro Pro Leu Asn Cys Ser Pro Asp Ser Leu Pro Val His Val | |
| gcc ggc cag tgc tgt aag gtc tgc cga cca aaa tgt atc tac gga ggg | 960 |
| Ala Gly Gln Cys Cys Lys Val Cys Arg Pro Lys Cys Ile Tyr Gly Gly | |
| aaa gtc ctt gca gaa ggc cag cgg att tta acc aag agc tgt cgg gaa | 1008 |
| Lys Val Leu Ala Glu Gly Gln Arg Ile Leu Thr Lys Ser Cys Arg Glu | |
| tgc cga ggt gga gtt tta gtg aaa att aca gaa gcg tgc cct cct ttg | 1056 |
| Cys Arg Gly Gly Val Leu Val Lys Ile Thr Glu Ala Cys Pro Pro Leu | |
| aac tgc tca gac aag gat cac att ctc cca gag aat cag tgc tgc agc | 1104 |
| Asn Cys Ser Asp Lys Asp His Ile Leu Pro Glu Asn Gln Cys Cys Ser | |
| gtc tgc aga ggt cat aac ttt tgt gcg gaa gga cct aaa tgt ggt gaa | 1152 |
| Val Cys Arg Gly His Asn Phe Cys Ala Glu Gly Pro Lys Cys Gly Glu | |
| aat tca gag tgc aaa aac tgg aat aca aaa gct act tgc gag tgc aag | 1200 |
| Asn Ser Glu Cys Lys Asn Trp Asn Thr Lys Ala Thr Cys Glu Cys Lys | |
| aat ggt tat atc tct gtc cag ggg gac tcc gcc tac tgt gaa gat atc | 1248 |
| Asn Gly Tyr Ile Ser Val Gln Gly Asp Ser Ala Tyr Cys Glu Asp Ile | |
| gat gag tgt gct gct aag atg cat tac tgt cgt gcc aat act gtg tgt | 1296 |
| Asp Glu Cys Ala Ala Lys Met His Tyr Cys Arg Ala Asn Thr Val Cys | |
| gtc aac ctg cct ggg tta tat cgg tgt gac tgt gtc ccg gga tac att | 1344 |
| Val Asn Leu Pro Gly Leu Tyr Arg Cys Asp Cys Val Pro Gly Tyr Ile | |
| cgc gtg gat gat ttc tct tgt aca gaa cat gac gaa tgt ggc agc ggg | 1392 |
| Arg Val Asp Asp Phe Ser Cys Thr Glu His Asp Glu Cys Gly Ser Gly | |
| cag cac aac tgt gat gag aat gcc atc tgc acc aac act gtc cag gga | 1440 |
| Gln His Asn Cys Asp Glu Asn Ala Ile Cys Thr Asn Thr Val Gln Gly | |
| cac agc tgc acc tgc aaa ccg ggc tac gtg ggg aat ggg acc agc tgc | 1488 |
| His Ser Cys Thr Cys Lys Pro Gly Tyr Val Gly Asn Gly Thr Ser Cys | |
| aga gcg ttc tgc gaa gag ggc tgc aga tat ggc ggg aca tgc gtg gct | 1536 |
| Arg Ala Phe Cys Glu Glu Gly Cys Arg Tyr Gly Gly Thr Cys Val Ala | |
| cct aac aaa tgt gtc tgt cct tct gga ttc aca gga agc cac tgt gag | 1584 |
| Pro Asn Lys Cys Val Cys Pro Ser Gly Phe Thr Gly Ser His Cys Glu | |
| aaa gac att gat gaa tgt gcc tta aga act cac acc tgt tgg aat gat | 1632 |
| Lys Asp Ile Asp Glu Cys Ala Leu Arg Thr His Thr Cys Trp Asn Asp | |
| tct gcc tgc atc aac ttg gca ggg ggc ttc gac tgc ctg tgt ccc tca | 1680 |
| Ser Ala Cys Ile Asn Leu Ala Gly Gly Phe Asp Cys Leu Cys Pro Ser | |
| ggg cca tcc tgc tct ggt gac tgc ccc cac gaa gga gga ctg aag cgc | 1728 |
| Gly Pro Ser Cys Ser Gly Asp Cys Pro His Glu Gly Gly Leu Lys Arg | |
| aac ggg cag gtg tgg acc ctg aaa gaa gac agg tgt tct gtg tgt tcc | 1776 |
| Asn Gly Gln Val Trp Thr Leu Lys Glu Asp Arg Cys Ser Val Cys Ser | |
| tgc aag gat ggg aag ata ttc tgc cga cgg aca gct tgt gat tgc cag | 1824 |
| Cys Lys Asp Gly Lys Ile Phe Cys Arg Arg Thr Ala Cys Asp Cys Gln | |
| aat cca agc gtt gac ctt ttc tgt tgc cca gag tgt gac acc agg gtc | 1872 |
| Asn Pro Ser Val Asp Leu Phe Cys Cys Pro Glu Cys Asp Thr Arg Val | |
| aca agt caa tgt tta gac caa aat gga cac aag ctc tat cga agt gga | 1920 |
| Thr Ser Gln Cys Leu Asp Gln Asn Gly His Lys Leu Tyr Arg Ser Gly | |
| gac aat tgg act cac agc tgt cag cag tgc cgg tgt ctg gaa gga gag | 1968 |
| Asp Asn Trp Thr His Ser Cys Gln Gln Cys Arg Cys Leu Glu Gly Glu | |
| gta gat tgc tgg cca ctc act tgc ccc aga ttg agc tgt gag tac aca | 2016 |
| Val Asp Cys Trp Pro Leu Thr Cys Pro Arg Leu Ser Cys Glu Tyr Thr | |
| gcc atc ttg gaa ggg gag tgt tgt cca cgc tgt gtc agc gac ccc tgc | 2064 |
| Ala Ile Leu Glu Gly Glu Cys Cys Pro Arg Cys Val Ser Asp Pro Cys | |
| ctg gcg gat aac atc gtc tat gac atc aga gaa act tgc ctg gac agc | 2112 |
| Leu Ala Asp Asn Ile Val Tyr Asp Ile Arg Glu Thr Cys Leu Asp Ser | |
| tat gga gtt tca agg ctt agt ggc tca gtg tgg aca ttg gct gga tct | 2160 |
| Tyr Gly Val Ser Arg Leu Ser Gly Ser Val Trp Thr Leu Ala Gly Ser | |
| ccc tgc acg acc tgc aaa tgc aag aat gga agt gtc tgc tgt tct gtg | 2208 |
| Pro Cys Thr Thr Cys Lys Cys Lys Asn Gly Ser Val Cys Cys Ser Val | |
| gat ttg gag tgt ctt cat aat aat tga aggatttaaa atggactcat | 2255 |
| Asp Leu Glu Cys Leu His Asn Asn * | |
| gatcgccaga gaaaaatgga caaatgacca | 2285 |

Equus caballus NELL1 isoform 2 amino acid sequence (SEQ ID NO: 8)
Met Gly Phe Gly Met Asp Pro Asp Leu gln Met Asp Ile Ile Thr Glu
Leu Asp Leu Val Asn Thr Thr Leu Gly Val Thr Gln Val Ser Gly Leu
His Asn Ala Ser Lys Ala Phe Leu Phe Gln Asp Val Glu Arg Glu Ile
His Ala Ala Pro His Val Ser Glu Lys Leu Ile Gln Leu Phe Arg Asn
Lys Ser Glu Phe Thr Phe Leu Ala Thr Val Gln Gln Lys Pro Ser Thr
Ser Gly Val Ile Leu Ser Ile Arg Glu Leu Glu Asn Ser Tyr Phe Glu
Leu Glu Ser Ser Gly Leu Arg Asp Glu Ile Arg Tyr His Tyr Thr His
Lys Gly Lys Pro Arg Thr Glu Ala Leu Pro Tyr Arg Met Ala Asp Gly
Arg Trp His Lys Val Ala Leu Ser Val Ser Ala Ser His Leu Leu Leu
His Ile Asp Cys Asn Arg Ile Tyr Glu Arg Val Ile Asp Thr Pro Glu
Thr Asn Leu Pro Pro Gly Ser Asn Leu Trp Leu Gly Gln Arg Asn Gln
Lys His Gly Leu Phe Lys Gly Ile Ile Gln Asp Gly Lys Ile Ile Phe
Met Pro Asn Gly Tyr Ile Thr Gln Cys Pro Asn Leu Asn Arg Thr Cys
Pro Thr Cys Ser Asp Phe Leu Ser Leu Val Gln Gly Ile Met Asp Leu
Gln Glu Leu Leu Ala Lys Met Thr Ala Lys Leu Asn Tyr Ala Glu Thr
Arg Leu Ser Gln Leu Glu Asn Cys His Cys Glu Lys Thr Cys Gln Val
Ser Gly Leu Leu Tyr Arg Asp Gln Asp Ser Trp Val Asp Gly Asp His
Cys Arg Asn Cys Thr Cys Lys Ser Gly Ala Val Glu Cys Arg Arg Met
Ser Cys Pro Pro Leu Asn Cys Ser Pro Asp Ser Leu Pro Val His Val
Ala Gly Gln Cys Cys Lys Val Cys Arg Pro Lys Cys Ile Tyr Gly Gly
Lys Val Leu Ala Glu Gly Gln Arg Ile Leu Thr Lys Ser Cys Arg Glu
Cys Arg Gly Gly Val Leu Val Lys Ile Thr Glu Ala Cys Pro Pro Leu
Asn Cys Ser Asp Lys Asp His Ile Leu Pro Glu Asn Gln Cys Cys Ser
Val Cys Arg Gly His Asn Phe Cys Ala Glu Gly Pro Lys Cys Gly Glu
Asn Ser Glu Cys Lys Asn Trp Asn Thr Lys Ala Thr Cys Glu Cys Lys
Asn Gly Tyr Ile Ser Val Gln Gly Asp Ser Ala Tyr Cys Glu Asp Ile
Asp Glu Cys Ala Ala Lys Met His Tyr Cys Arg Ala Asn Thr Val Cys
Val Asn Leu Pro Gly Leu Tyr Arg Cys Asp Cys Val Pro Gly Tyr Ile
Arg Val Asp Asp Phe Ser Cys Thr Glu His Asp Glu Cys Gly Ser Gly
Gln His Asn Cys Asp Glu Asn Ala Ile cys Thr Asn Thr Val Gln Gly
His Ser Cys Thr Cys Lys Pro Gly Tyr Val Gly Asn Gly Thr Ser Cys
Arg Ala Phe Cys Glu Glu Gly Cys Arg Tyr Gly Gly Thr Cys Val Ala
Pro Asn Lys Cys Val Cys Pro Ser Gly Phe Thr Gly Ser His Cys Glu
Lys Asp Ile Asp Glu Cys Ala Leu Arg Thr His Thr Cys Trp Asn Asp
Ser Ala Cys Ile Asn Leu Ala Gly Gly Phe Asp Cys Leu Cys Pro Ser
Gly Pro Ser Cys Ser Gly Asp Cys Pro His Glu Gly Gly Leu Lys Arg
Asn Gly Gln Val Trp Thr Leu Lys Glu Asp Arg Cys Ser Val Cys Ser
Cys Lys Asp Gly Lys Ile Phe Cys Arg Arg Thr Ala Cys Asp Cys Gln
Asn Pro Ser Val Asp Leu Phe Cys Cys Pro Glu Cys Asp Thr Arg Val
Thr Ser Gln Cys Leu Asp Gln Asn Gly His Lys Leu Tyr Arg Ser Gly
Asp Asn Trp Thr His Ser Cys Gln Gln Cys Arg Cys Leu Glu Gly Glu
Val Asp Cys Trp Pro Leu Thr Cys Pro Arg Leu Ser Cys Glu Tyr Thr
Ala Ile Leu Glu Gly Glu Cys Cys Pro Arg Cys Val Ser Asp Pro Cys
Leu Ala Asp Asn Ile Val Tyr Asp Ile Arg Glu Thr Cys Leu Asp Ser
Tyr Gly Val Ser Arg Leu Ser Gly Ser Val Trp Thr Leu Ala Gly Ser
Pro Cys Thr Thr Cys Lys Cys Lys Asn Gly Ser Val Cys Cys Ser Val
Asp Leu Glu Cys Leu His Asn Asn
Mus musculus NELL1 nucleotide sequence (SEQ ID NO: 9) and translated;
ID NO: 10)

| gcgttggtgc gccctgcttg gcggggggcc tccggagcg atg ccg atg gat gtg | 54 | |
|---|---|---|
| Met Pro Met Asp Val | | |
| att tta gtt ttg tgg ttc tgt gtg tgc acc gcc agg aca gtg ctg ggc | 102 | |
| Ile Leu Val Leu Trp Phe Cys Val Cys Thr Ala Arg Thr Val Leu Gly | | |
| ttt ggg atg gac cct gac ctt cag atg gac atc atc act gaa ctt gac | 150 | |
| Phe Gly Met Asp Pro Asp Leu Gln Met Asp Ile Ile Thr Glu Leu Asp | | |
| ctt gtg aac acc acc ctg ggc gtc act cag gtg gct gga cta cac aat | 198 | |
| Leu Val Asn Thr Thr Leu Gly Val Thr Gln Val Ala Gly Leu His Asn | | |
| gcc agt aag gca ttt ctg ttt caa gat gta cag aga gag atc cac tca | 246 | |
| Ala Ser Lys Ala Phe Leu Phe Gln Asp Val Gln Arg Glu Ile His Ser | | |
| gcc cct cat gtg agt gag aag ctg atc cag cta ttc cgg aat aag agt | 294 | |
| Ala Pro His Val Ser Glu Lys Leu Ile Gln Leu Phe Arg Asn Lys Ser | | |
| gag ttt acc ttt ttg gct aca gtg cag cag aag ccg tcc acc tca ggg | 342 | |
| Glu Phe Thr Phe Leu Ala Thr Val Gln Gln Lys Pro Ser Thr Ser Gly | | |
| gtg ata ctg tcg atc cgg gag ctg gaa cac agc tat ttt gaa ctg gag | 390 | |
| Val Ile Leu Ser Ile Arg Glu Leu Glu His Ser Tyr Phe Glu Leu Glu | | |
| agc agt ggc cca aga gaa gag ata cgc tat cat tac atc cat ggc ggc | 438 | |
| Ser Ser Gly Pro Arg Glu Glu Ile Arg Tyr His Tyr Ile His Gly Gly | | |
| aag ccc agg act gag gcc ctt ccc tac cgc atg gcc gat gga cag tgg | 486 | |
| Lys Pro Arg Thr Glu Ala Leu Pro Tyr Arg Met Ala Asp Gly Gln Trp | | |
| cac aag gtc gcg ctg tct gtg agc gcc tct cac ctc cta ctc cat gtc | 534 | |
| His Lys Val Ala Leu Ser Val Ser Ala Ser His Leu Leu Leu His Val | | |
| gac tgc aat agg att tat gag cgt gtg ata gat cct ccg gag acc aac | 582 | |
| Asp Cys Asn Arg Ile Tyr Glu Arg Val Ile Asp Pro Pro Glu Thr Asn | | |
| ctt cct cca gga agc aat cta tgg ctt ggg caa cgt aat caa aag cat | 630 | |
| Leu Pro Pro Gly Ser Asn Leu Trp Leu Gly Gln Arg Asn Gln Lys His | | |
| ggc ttt ttc aaa gga atc atc caa gat ggc aag atc atc ttc atg ccg | 678 | |
| Gly Phe Phe Lys Gly Ile Ile Gln Asp Gly Lys Ile Ile Phe Met Pro | | |
| aac ggc ttc atc aca cag tgc ccc aac cta aat cgc act tgc cca aca | 726 | |
| Asn Gly Phe Ile Thr Gln Cys Pro Asn Leu Asn Arg Thr Cys Pro Thr | | |
| tgc agt gat ttc ctg agc ctg gtt caa gga ata atg gat ttg caa gag | 774 | |
| Cys Ser Asp Phe Leu Ser Leu Val Gln Gly Ile Met Asp Leu Gln Glu | | |
| ctt ttg gcc aag atg act gca aaa ctg aat tat gca gag acg aga ctt | 822 | |
| Leu Leu Ala Lys Met Thr Ala Lys Leu Asn Tyr Ala Glu Thr Arg Leu | | |
| ggt caa ctg gaa aat tgc cac tgt gag aag acc tgc caa gtg agt ggg | 870 | |
| Gly Gln Leu Glu Asn Cys His Cys Glu Lys Thr Cys Gln Val Ser Gly | | |
| ctg ctc tac agg gac caa gac tcc tgg gta gat ggt gac aac tgc agg | 918 | |
| Leu Leu Tyr Arg Asp Gln Asp Ser Trp Val Asp Gly Asp Asn Cys Arg | | |
| aac tgc aca tgc aaa agt ggt gct gtg gag tgc cga agg atg tcc tgt | 966 | |
| Asn Cys Thr Cys Lys Ser Gly Ala Val Glu Cys Arg Arg Met Ser Cys | | |
| ccc cca ctc aac tgt tcc cca gac tca ctt cct gtg cat att tct ggc | 1014 | |
| Pro Pro Leu Asn Cys Ser Pro Asp Ser Leu Pro Val His Ile Ser Gly | | |
| caa tgt tgt aaa gtt tgc aga cca aaa tgt atc tat gga gga aaa gtt | 1062 | |
| Gln Cys Cys Lys Val Cys Arg Pro Lys Cys Ile Tyr Gly Gly Lys Val | | |
| ctt gct gag ggc cag cgg att tta acc aag acc tgc cgg gaa tgt cga | 1110 | |
| Leu Ala Glu Gly Gln Arg Ile Leu Thr Lys Thr Cys Arg Glu Cys Arg | | |
| ggt gga gtc ttg gta aaa atc aca gaa gct tgc cct cct ttg aac tgc | 1158 | |
| Gly Gly Val Leu Val Lys Ile Thr Glu Ala Cys Pro Pro Leu Asn Cys | | |
| tca gag aag gat cat att ctt ccg gag aac cag tgc tgc agg gtc tgc | 1206 | |
| Ser Glu Lys Asp His Ile Leu Pro Glu Asn Gln Cys Cys Arg Val Cys | | |
| cga ggt cat aac ttc tgt gca gaa gca cct aag tgt gga gaa aac tcg | 1254 | |
| Arg Gly His Asn Phe Cys Ala Glu Ala Pro Lys Cys Gly Glu Asn Ser | | |
| gaa tgc aaa aat tgg aat aca aaa gcg act tgt gag tgc aag aat gga | 1302 | |
| Glu Cys Lys Asn Trp Asn Thr Lys Ala Thr Cys Glu Cys Lys Asn Gly | | |
| tac atc tct gtc cag ggc aac tct gca tac tgt gaa gat atc gat gag | 1350 | |
| Tyr Ile Ser Val Gln Gly Asn Ser Ala Tyr Cys Glu Asp Ile Asp Glu | | |
| tgt gca gca aag atg cac tac tgt cat gcc aac acg gtg tgt gtc aac | 1398 | |
| Cys Ala Ala Lys Met His Tyr Cys His Ala Asn Thr Val Cys Val Asn | | |
| ttg ccg ggg tta tat cgc tgt gac tgc atc cca gga tac atc cgt gtg | 1446 | |
| Leu Pro Gly Leu Tyr Arg Cys Asp Cys Ile Pro Gly Tyr Ile Arg Val | | |
| gat gac ttc tct tgt acg gag cat gat gat tgt ggc agc gga caa cac | 1494 | |
| Asp Asp Phe Ser Cys Thr Glu His Asp Asp Cys Gly Ser Gly Gln His | | |
| aac tgt gac aaa aat gcc atc tgt acc aac aca gtc cag gga cac agc | 1542 | |
| Asn Cys Asp Lys Asn Ala Ile Cys Thr Asn Thr Val Gln Gly His Ser | | |
| tgt acc tgc cag cca ggc tac gtg gga aat ggt act gtc tgc aaa gca | 1590 | |
| Cys Thr Cys Gln Pro Gly Tyr Val Gly Asn Gly Thr Val Cys Lys Ala | | |
| ttc tgt gaa gag ggt tgc aga tac gga ggt acc tgt gtg gcc cct aac | 1638 | |
| Phe Cys Glu Glu Gly Cys Arg Tyr Gly Gly Thr Cys Val Ala Pro Asn | | |
| aaa tgt gtc tgt cct tct gga ttc aca gga agc cac tgt gag aaa gat | 1686 | |
| Lys Cys Val Cys Pro Ser Gly Phe Thr Gly Ser His Cys Glu Lys Asp | | |
| att gat gaa tgt gca gag gga ttc gtt gag tgc cac aac cac tcc cgc | 1734 | |
| Ile Asp Glu Cys Ala Glu Gly Phe Val Glu Cys His Asn His Ser Arg | | |
| tgc gtt aac ctt cca ggg tgg tac cac tgt gag tgc aga agc ggt ttc | 1782 | |
| Cys Val Asn Leu Pro Gly Trp Tyr His Cys Glu Cys Arg Ser Gly Phe | | |
| cat gac gat ggg acc tat tca ctg tcc ggg gag tcc tgc att gat att | 1830 | |
| His Asp Asp Gly Thr Tyr Ser Leu Ser Gly Glu Ser Cys Ile Asp Ile | | |
| gat gaa tgt gcc tta aga act cac act tgt tgg aat gac tct gcc tgc | 1878 | |
| Asp Glu Cys Ala Leu Arg Thr His Thr Cys Trp Asn Asp Ser Ala Cys | | |
| atc aac tta gca gga gga ttt gac tgc ctg tgt ccc tct ggg ccc tcc | 1926 | |
| Ile Asn Leu Ala Gly Gly Phe Asp Cys Leu Cys Pro Ser Gly Pro Ser | | |
| tgc tct ggt gac tgt ccc cac gaa ggg ggg ctg aag cat aat ggg cag | 1974 | |
| Cys Ser Gly Asp Cys Pro His Glu Gly Gly Leu Lys His Asn Gly Gln | | |
| gtg tgg att ctg aga gaa gac agg tgt tca gtc tgt tcc tgt aag gat | 2022 | |
| Val Trp Ile Leu Arg Glu Asp Arg Cys Ser Val Cys Ser Cys Lys Asp | | |
| ggg aag ata ttc tgc cgg cgg aca gct tgt gat tgc cag aat cca aat | 2070 | |
| Gly Lys Ile Phe Cys Arg Arg Thr Ala Cys Asp Cys Gln Asn Pro Asn | | |
| gtt gac ctt ttc tgc tgc cca gag tgt gac acc agg gtc act agc caa | 2118 | |
| Val Asp Leu Phe Cys Cys Pro Glu Cys Asp Thr Arg Val Thr Ser Gln | | |
| tgt tta gat caa agc gga cag aag ctc tat cga agt gga gac aac tgg | 2166 | |
| Cys Leu Asp Gln Ser Gly Gln Lys Leu Tyr Arg Ser Gly Asp Asn Trp | | |
| acc cac agc tgc cag cag tgc cga tgt ctg gaa gga gag gca gac tgc | 2214 | |
| Thr His Ser Cys Gln Gln Cys Arg Cys Leu Glu Gly Glu Ala Asp Cys | | |
| tgg cct cta gct tgc cct agt ttg agc tgt gaa tac aca gcc atc ttt | 2262 | |
| Trp Pro Leu Ala Cys Pro Ser Leu Ser Cys Glu Tyr Thr Ala Ile Phe | | |
| gaa gga gag tgt tgt ccc cgc tgt gtc agt gac ccc tgc ctg gct gat | 2310 | |
| Glu Gly Glu Cys Cys Pro Arg Cys Val Ser Asp Pro Cys Leu Ala Asp | | |
| aat att gcc tat gac atc aga aaa act tgc ctg gac agc tct ggt att | 2358 | |
| Asn Ile Ala Tyr Asp Ile Arg Lys Thr Cys Leu Asp Ser Ser Gly Ile | | |
| tcg agg ctg agc ggc gca gtg tgg aca atg gct gga tct ccc tgt aca | 2406 | |
| Ser Arg Leu Ser Gly Ala Val Trp Thr Met Ala Gly Ser Pro Cys Thr | | |
| acc tgt caa tgc aag aat ggg aga gtc tgc tgc tct gtg gat ctg gtg | 2454 | |
| Thr Cys Gln Cys Lys Asn Gly Arg Val Cys Cys Ser Val Asp Leu Val | | |
| tgt ctt gag aat aac tga agattttaaa tggactcatc acatgagaaa | 2502 | |
| Cys Leu Glu Asn Asn * | | |
| atggacaaaa tgaccatcca acctgaggaa gaggaggggc tgatttcttt ttctttttaa | 2562 | |
| ccacagtcaa ttaccaaagt ctccatcaga ggaaggcgtt tgggttgcct ttaccacttt | 2622 | |
| gctcatcctt gctgacctag tctagatgcc tgcagtaccg tgtatttcgg tcgatggttg | 2682 | |
| ttgagtctcc gtgctgtaaa tcacatttcc cttgtcagat catttacaga tacatttaaa | 2742 | |
| ggattccatg ataaatgtta aagtaccttt tgtttatttt gtgtaccaac ataatagaga | 2802 | |
| cttggcacca | | 2812 |

Mus musculus NELL1 amino acid sequence (SEQ ID NO: 10)
Met Pro Met Asp Val Ile Leu Val Leu Trp Phe Cys Val Cys Thr Ala
Arg Thr Val Leu Gly Phe Gly Met Asp Pro Asp Leu Gln Met Asp Ile
Ile Thr Glu Leu Asp Leu Val Asn Thr Thr Leu Gly Val Thr Gln Val
Ala Gly Leu His Asn Ala Ser Lys Ala Phe Leu Phe Gln Asp Val Gln
Arg Glu Ile His Ser Ala Pro His Val Ser Glu Lys Leu Ile Gln Leu
Phe Arg Asn Lys Ser Glu Phe Thr Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Pro Arg Glu Glu Ile Arg Tyr His
Tyr Ile His Gly Gly Lys Pro Arg Thr Glu Ala Leu Pro Tyr Arg Met
Ala Asp Gly Gln Trp His Lys Val Ala Leu Ser Val Ser Ala Ser His
Leu Leu Leu His Val Asp Cys Asn Arg Ile Tyr Glu Arg Val Ile Asp
Pro Pro Glu Thr Asn Leu Pro Pro Gly Ser Asn Leu Trp Leu Gly Gln
Arg Asn Gln Lys His Gly Phe Phe Lys Gly Ile Ile Gln Asp Gly Lys
Ile Ile Phe Met Pro Asn Gly Phe Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Gly Gln Leu Glu Asn Cys His Cys Glu Lys Thr
Cys Gln Val Ser Gly Leu Leu Tyr Arg Asp Gln Asp Ser Trp Val Asp
Gly Asp Asn Cys Arg Asn Cys Thr Cys Lys Ser Gly Ala Val Glu Cys
Arg Arg Met Ser Cys Pro Pro Leu Asn Cys Ser Pro Asp Ser Leu Fro
Val His Ile Ser Gly Gln Cys Cys Lys Val Cys Arg Pro Lys Cys Ile
Tyr Gly Gly Lys Val Leu Ala Glu Gly Gln Arg Ile Leu Thr Lys Thr
Cys Arg Glu Cys Arg Gly Gly Val Leu Val Lys Ile Thr Glu Ala Cys
Pro Pro Leu Asn Cys Ser Glu Lys Asp His Ile Leu Pro Glu Asn Gln
Cys Cys Arg Val Cys Arg Gly His Asn Phe Cys Ala Glu Ala Pro Lys
Cys Gly Glu Asn Ser Glu Cys Lys Asn Trp Asn Thr Lys Ala Thr Cys
Glu Cys Lys Asn Gly Tyr Ile Ser Val Gln Gly Asn Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Ile Pro
Gly Tyr Ile Arg Val Asp Asp Phe Ser Cys Thr Glu His Asp Asp Cys
Gly Ser Gly Gln His Asn Cys Asp Lys Asn Ala Ile Cys Thr Asn Thr
Val Gln Gly His Ser Cys Thr Cys Gln Pro Gly Tyr Val Gly Asn Gly
Thr Val Cys Lys Ala Phe Cys Glu Glu Gly Cys Arg Tyr Gly Gly Thr
Cys Val Ala Pro Asn Lys Cys Val Cys Pro Ser Gly Phe Thr Gly Ser
His Cys Glu Lys Asp Ile Asp Glu Cys Ala Glu Gly Phe Val Glu Cys
His Asn His Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Ile Asp Ile Asp Glu Cys Ala Leu Arg Thr His Thr Cys Trp
Asn Asp Ser Ala Cys Ile Asn Leu Ala Gly Gly Phe Asp Cys Leu Cys
Pro Ser Gly Pro Ser Cys Ser Gly Asp Cys Pro His Glu Gly Gly Leu
Lys His Asn Gly Gln Val Trp Ile Leu Arg Glu Asp Arg Cys Ser Val
Cys Ser Cys Lys Asp Gly Lys Ile Phe Cys Arg Arg Thr Ala Cys Asp
Cys Gln Asn Pro Asn Val Asp Leu Phe Cys Cys Pro Glu Cys Asp Thr
Arg Val Thr Ser Gln Cys Leu Asp Gln Ser Gly Gln Lys Leu Tyr Arg
Ser Gly Asp Asn Trp Thr His Ser Cys Gln Gln Cys Arg Cys Leu Glu
Gly Glu Ala Asp Cys Trp Pro Leu Ala Cys Pro Ser Leu Ser Cys Glu
Tyr Thr Ala Ile Phe Glu Gly Glu Cys Cys Pro Arg Cys Val Ser Asp
Pro Cys Leu Ala Asp Asn Ile Ala Tyr Asp Ile Arg Lys Thr Cys Leu
Asp Ser Ser Gly Ile Ser Arg Leu Ser Gly Ala Val Trp Thr Met Ala
Gly Ser Pro Cys Thr Thr Cys Gln Cys Lys Asn Gly Arg Val Cys Cys
Ser Val Asp Leu Val Cys Leu Glu Asn Asn
Rattus norvesicus NELL1 nucleotide sequence (SEQ ID NO: 11) and translated
amino acid sequence (SEQ ID NO: 12)

| | |
|---|---|
| aagcactggt ttcttgttag cgttggtgcg ccctgcttgg cgggggttct ccggagcg | 58 |
| atg ccg atg gat gtg att tta gtt ttg tgg ttc tgt gta tgc acc gcc | 106 |
| Met Pro Met Asp Val Ile Leu Val Leu Trp Phe Cys Val Cys Thr Ala | |
| agg aca gtg ttg ggc ttt ggg atg gac cct gac ctt cag ctg gac atc | 154 |
| Arg Thr Val Leu Gly Phe Gly Met Asp Pro Asp Leu Gln Leu Asp Ile | |
| atc tca gag ctc gac ctg gtg aac acc acc ctg gga gtc acg cag gtg | 202 |
| Ile Ser Glu Leu Asp Leu Val Asn Thr Thr Leu Gly Val Thr Gln Val | |
| gct gga ctg cac aac gcc agt aaa gca ttt cta ttt caa gat gta cag | 250 |
| Ala Gly Leu His Asn Ala Ser Lys Ala Phe Leu Phe Gln Asp Val Gln | |
| aga gag atc cat tcg gcc cct cac gtg agt gag aag ctg atc cag cta | 298 |
| Arg Glu Ile His Ser Ala Pro His Val Ser Glu Lys Leu Ile Gln Leu | |
| ttc cgg aat aag agc gag ttc acc ttt ttg gct aca gtg cag cag aaa | 346 |
| Phe Arg Asn Lys Ser Glu Phe Thr Phe Leu Ala Thr Val Gln Gln Lys | |
| cca tcc acc tca ggg gtg ata ctg tcc atc cgg gag ctg gag cac agc | 394 |
| Pro Ser Thr Ser Gly Val Ile Leu Ser Ile Arg Glu Leu Glu His Ser | |
| tat ttt gaa ctg gag agc agt ggc cca aga gaa gag ata cgc tac cat | 442 |
| Tyr Phe Glu Leu Glu Ser Ser Gly Pro Arg Glu Glu Ile Arg Tyr His | |
| tac ata cat ggt gga aag ccc agg act gag gcc ctt ccc tac cgc atg | 490 |
| Tyr Ile His Gly Gly Lys Pro Arg Thr Glu Ala Leu Pro Tyr Arg Met | |
| gca gac gga caa tgg cac aag gtc gcg ctg tca gtg agc gcc tct cac | 538 |
| Ala Asp Gly Gln Trp His Lys Val Ala Leu Ser Val Ser Ala Ser His | |
| ctc ctg ctc cac atc gac tgc aat agg att tac gag cgt gtg ata gac | 586 |
| Leu Leu Leu His Ile Asp Cys Asn Arg Ile Tyr Glu Arg Val Ile Asp | |
| cct ccg gag acc aac ctt cct cca gga agc aat ctg tgg ctt ggg caa | 634 |
| Pro Pro Glu Thr Asn Leu Pro Pro Gly Ser Asn Leu Trp Leu Gly Gln | |
| cgt aac caa aag cat ggc ttt ttc aaa gga atc atc caa gat ggt aag | 682 |
| Arg Asn Gln Lys His Gly Phe Phe Lys Gly Ile Ile Gln Asp Gly Lys | |
| atc atc ttc atg ccg aat ggt ttc atc aca cag tgt ccc aac ctc aat | 730 |
| Ile Ile Phe Met Pro Asn Gly Phe Ile Thr Gln Cys Pro Asn Leu Asn | |
| cgc act tgc cca aca tgc agt gac ttc ctg agc ctg gtt caa gga ata | 778 |
| Arg Thr Cys Pro Thr Cys Ser Asp Phe Leu Ser Leu Val Gln Gly Ile | |
| atg gat ttg caa gag ctt ttg gcc aag atg act gca aaa ctg aat tat | 826 |
| Met Asp Leu Gln Glu Leu Leu Ala Lys Met Thr Ala Lys Leu Asn Tyr | |
| gca gag acg aga ctt ggt caa ctg gaa aat tgc cac tgt gag aag acc | 874 |
| Ala Glu Thr Arg Leu Gly Gln Leu Glu Asn Cys His Cys Glu Lys Thr | |
| tgc caa gtg agt ggg ctg ctc tac agg gac caa gac tcc tgg gtg gat | 922 |
| Cys Gln Val Ser Gly Leu Leu Tyr Arg Asp Gln Asp Ser Trp Val Asp | |
| ggt gac aac tgt ggg aac tgc acg tgc aaa agt ggt gcc gtg gag tgc | 970 |
| Gly Asp Asn Cys Gly Asn Cys Thr Cys Lys Ser Gly Ala Val Glu Cys | |
| cgc agg atg tcc tgt ccc ccg ctc aac tgt tcc ccg gac tca ctt cct | 1018 |
| Arg Arg Met Ser Cys Pro Pro Leu Asn Cys Ser Pro Asp Ser Leu Pro | |
| gtg cac att tcc ggc cag tgt tgt aaa gtt tgc aga cca aaa tgt atc | 1066 |
| Val His Ile Ser Gly Gln Cys Cys Lys Val Cys Arg Pro Lys Cys Ile | |
| tat gga gga aaa gtt ctt gct gag ggc cag cgg att tta acc aag acc | 1114 |
| Tyr Gly Gly Lys Val Leu Ala Glu Gly Gln Arg Ile Leu Thr Lys Thr | |
| tgc cgg gaa tgt cga ggt gga gtc ttg gta aaa atc aca gaa gct tgc | 1162 |
| Cys Arg Glu Cys Arg Gly Gly Val Leu Val Lys Ile Thr Glu Ala Cys | |
| cct cct ttg aac tgc tca gca aag gat cat att ctt cca gag aat cag | 1210 |
| Pro Pro Leu Asn Cys Ser Ala Lys Asp His Ile Leu Pro Glu Asn Gln | |
| tgc tgc agg gtc tgc cca ggt cat aac ttc tgt gca gaa gca cct aag | 1258 |
| Cys Cys Arg Val Cys Pro Gly His Asn Phe Cys Ala Glu Ala Pro Lys | |
| tgc gga gaa aac tcg gaa tgc aaa aat tgg aat aca aaa gca acc tgt | 1306 |
| Cys Gly Glu Asn Ser Glu Cys Lys Asn Trp Asn Thr Lys Ala Thr Cys | |
| gag tgc aag aat gga tac atc tct gtc cag ggc aac tct gca tac tgt | 1354 |
| Glu Cys Lys Asn Gly Tyr Ile Ser Val Gln Gly Asn Ser Ala Tyr Cys | |
| gaa gat att gat gag tgt gca gct aaa atg cac tat tgt cat gcc aac | 1402 |
| Glu Asp Ile Asp Glu Cys Ala Ala Lys Met His Tyr Cys His Ala Asn | |
| acc gtg tgt gtc aac ttg ccg ggg ttg tat cgc tgt gac tgc gtc cca | 1450 |
| Thr Val Cys Val Asn Leu Pro Gly Leu Tyr Arg Cys Asp Cys Val Pro | |
| ggg tac atc cgt gtg gat gac ttc tct tgt acg gag cat gat gat tgt | 1498 |
| Gly Tyr Ile Arg Val Asp Asp Phe Ser Cys Thr Glu His Asp Asp Cys | |
| ggc agc gga caa cac aac tgc gac aaa aat gcc atc tgt acc aac aca | 1546 |
| Gly Ser Gly Gln His Asn Cys Asp Lys Asn Ala Ile Cys Thr Asn Thr | |
| gtc cag gga cac agc tgc acc tgc cag ccg ggt tac gtg gga aat ggc | 1594 |
| Val Gln Gly His Ser Cys Thr Cys Gln Pro Gly Tyr Val Gly Asn Gly | |
| acc atc tgc aaa gca ttc tgt gaa gag ggt tgc aga tac gga ggt acc | 1642 |
| Thr Ile Cys Lys Ala Phe Cys Glu Glu Gly Cys Arg Tyr Gly Gly Thr | |
| tgt gtg gct cct aac aag tgt gtc tgt cct tct gga ttc acg gga agc | 1690 |
| Cys Val Ala Pro Asn Lys Cys Val Cys Pro Ser Gly Phe Thr Gly Ser | |
| cac tgt gag aaa gat att gat gaa tgc gca gag gga ttc gtt gaa tgc | 1738 |
| His Cys Glu Lys Asp Ile Asp Glu Cys Ala Glu Gly Phe Val Glu Cys | |
| cac aac tac tcc cgc tgt gtt aac ctg cca ggg tgg tac cac tgt gag | 1786 |
| His Asn Tyr Ser Arg Cys Val Asn Leu Pro Gly Trp Tyr His Cys Glu | |
| tgc aga agc ggt ttc cat gac gat ggg acc tac tca ctg tcc ggg gag | 1834 |
| Cys Arg Ser Gly Phe His Asp Asp Gly Thr Tyr Ser Leu Ser Gly Glu | |
| tcc tgc att gat atc gat gaa tgt gcc tta aga act cac act tgt tgg | 1882 |
| Ser Cys Ile Asp Ile Asp Glu Cys Ala Leu Arg Thr His Thr Cys Trp | |
| aat gac tct gcc tgc atc aac tta gca gga gga ttt gac tgc ctg tgt | 1930 |
| Asn Asp Ser Ala Cys Ile Asn Leu Ala Gly Gly Phe Asp Cys Leu Cys | |
| ccc tct ggg ccc tcc tgc tct ggt gac tgt ccc cac gaa gga ggg ctg | 1978 |
| Pro Ser Gly Pro Ser Cys Ser Gly Asp Cys Pro His Glu Gly Gly Leu | |
| aag cat aat ggg cag gtg tgg att ctg aga gaa gac agg tgt tca gtc | 2026 |
| Lys His Asn Gly Gln Val Trp Ile Leu Arg Glu Asp Arg Cys Ser Val | |
| tgt tcc tgc aag gat ggg aag ata ttc tgc cgg cgg aca gct tgt gat | 2074 |
| Cys Ser Cys Lys Asp Gly Lys Ile Phe Cys Arg Arg Thr Ala Cys Asp | |
| tgc cag aat cca aat gtt gac ctt ttt tgc tgc cca gag tgc gat acc | 2122 |
| Cys Gln Asn Pro Asn Val Asp Leu Phe Cys Cys Pro Glu Cys Asp Thr | |
| agg gtc acc agc caa tgt tta gat caa agt gga cag aag ctc tat cga | 2170 |
| Arg Val Thr Ser Gln Cys Leu Asp Gln Ser Gly Gln Lys Leu Tyr Arg | |
| agt gga gac aac tgg acc cac agc tgc cag cag tgc cga tgt ctg gaa | 2218 |
| Ser Gly Asp Asn Trp Thr His Ser Cys Gln Gln Cys Arg Cys Leu Glu | |
| gga gag gca gac tgc tgg cct ctg gct tgc cct agt ttg ggc tgt gaa | 2266 |
| Gly Glu Ala Asp Cys Trp Pro Leu Ala Cys Pro Ser Leu Gly Cys Glu | |
| tac aca gcc atg ttt gaa ggg gag tgt tgt ccc cga tgt gtc agt gac | 2314 |
| Tyr Thr Ala Met Phe Glu Gly Glu Cys Cys Pro Arg Cys Val Ser Asp | |
| ccc tgc ctg gct ggt aat att gcc tat gac atc aga aaa act tgc ctg | 2362 |
| Pro Cys Leu Ala Gly Asn Ile Ala Tyr Asp Ile Arg Lys Thr Cys Leu | |
| gac agc ttt ggt gtt tcg agg ctg agc gga gcc gtg tgg aca atg gct | 2410 |
| Asp Ser Phe Gly Val Ser Arg Leu Ser Gly Ala Val Trp Thr Met Ala | |
| gga tct cct tgt aca acc tgc aaa tgc aag aat ggg aga gtc tgc tgc | 2458 |
| Gly Ser Pro Cys Thr Thr Cys Lys Cys Lys Asn Gly Arg Val Cys Cys | |
| tct gtg gat ctg gag tgt att gag aat aac tga agattttaaa tggactcgtc | 2511 |
| Ser Val Asp Leu Glu Cys Ile Glu Asn Asn * | |
| acgtgagaaa atgggcaaaa tgatcatccc acctgaggaa gaagaggggc tgatttcttt | 2571 |
| ttctttttaa ccacagtcaa ttaccaaagt ctccatctga ggaaggcgtt tggattgcct | 2631 |
| ttgccacttt gctcatcctt gctgacctag tctagatgcc tgcagtaccg tgcatttcgg | 2691 |
| tcgatggttg ttgagtctca gtgttgtaaa tcgcatttcc ctcgtcagat catttacaga | 2751 |
| tacatttaaa ggggttccat gataaatgtt aatgtaactt ttgtttattt tgtgtactga | 2811 |
| cataatagag acttggcacc atttatttat ttttcttgat ttttggatca aattctaaaa | 2871 |
| ataaagttgc ctgttgcgaa aaaaaaaaaa aaaaaaaaaa aaaa | 2915 |

Rattus norvegicus NELL1 amino acid sequence (SEQ ID NO: 12)
Met Pro Met Asp Val Ile Leu Val Leu Trp Phe Cys Val Cys Thr Ala
Arg Thr Val Leu Gly Phe Gly Met Asp Pro Asp Leu Gln Leu Asp Ile
Ile Ser Glu Leu Asp Leu Val Asn Thr Thr Leu Gly Val Thr Gln Val
Ala Gly Leu His Asn Ala Ser Lys Ala Phe Leu Phe Gln Asp Val Gln
Arg Glu Ile His Ser Ala Pro His Val Ser Glu Lys Leu Ile Gln Leu
Phe Arg Asn Lys Ser Glu Phe Thr Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Pro Arg Glu Glu Ile Arg Tyr His
Tyr Ile His Gly Gly Lys Pro Arg Thr Glu Ala Leu Pro Tyr Arg Met
Ala Asp Gly Gln Trp His Lys Val Ala Leu Ser Val Ser Ala Ser His
Leu Leu Leu His Ile Asp Cys Asn Arg Ile Tyr Glu Arg Val Ile Asp
Pro Pro Glu Thr Asn Leu Pro Pro Gly Ser Asn Leu Trp Leu Gly Gln
Arg Asn Gln Lys His Gly Phe Phe Lys Gly Ile Ile Gln Asp Gly Lys
Ile Ile Phe Met Pro Asn Gly Phe Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Gly Gln Leu Glu Asn Cys His Cys Glu Lys Thr
Cys Gln Val Ser Gly Leu Leu Tyr Arg Asp Gln Asp Ser Trp Val Asp
Gly Asp Asn Cys Gly Asn Cys Thr Cys Lys Ser Gly Ala Val Glu Cys
Arg Arg Met Ser Cys Pro Pro Leu Asn Cys Ser Pro Asp Ser Leu Pro
Val His Ile Ser Gly Gln Cys Cys Lys Val Cys Arg Pro Lys Cys Ile
Tyr Gly Gly Lys Val Leu Ala Glu Gly Gln Arg Ile Leu Thr Lys Thr
Cys Arg Glu Cys Arg Gly Gly Val Leu Val Lys Ile Thr Glu Ala Cys
Pro Pro Leu Asn Cys Ser Ala Lys Asp His Ile Leu Pro Glu Asn Gln
Cys Cys Arg Val Cys Pro Gly His Asn Phe Cys Ala Glu Ala Pro Lys
Cys Gly Glu Asn Ser Glu Cys Lys Asn Trp Asn Thr Lys Ala Thr Cys
Glu Cys Lys Asn Gly Tyr Ile Ser Val Gln Gly Asn Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Fro
Gly Tyr Ile Arg Val Asp Asp Phe Ser Cys Thr Glu His Asp Asp Cys
Gly Ser Gly Gln His Asn Cys Asp Lys Asn Ala Ile Cys Thr Asn Thr
Val Gln Gly His Ser Cys Thr Cys Gln Pro Gly Tyr Val Gly Asn Gly
Thr Ile Cys Lys Ala Phe Cys Glu Glu Gly Cys Arg Tyr Gly Gly Thr
Cys Val Ala Pro Asn Lys Cys Val Cys Pro Ser Gly Phe Thr Gly Ser
His Cys Glu Lys Asp Ile Asp Glu Cys Ala Glu Gly Phe Val Glu Cys
His Asn Tyr Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Ile Asp Ile Asp Glu Cys Ala Leu Arg Thr His Thr Cys Trp
Asn Asp Ser Ala Cys Ile Asn Leu Ala gly Gly Phe Asp Cys Leu Cys
Pro Ser Gly Pro Ser Cys Ser Gly Asp Cys Pro His glu Gly Gly Leu
Lys His Asn Gly Gln Val Trp Ile Leu Arg Glu Asp Arg cys Ser Val
Cys Ser Cys Lys Asp Gly Lys Ile Phe Cys Arg Arg Thr Ala Cys Asp
Cys Gln Asn Pro Asn Val Asp Leu Phe Cys Cys Pro Glu Cys Asp Thr
Arg Val Thr Ser Gln Cys Leu Asp Gln Ser Gly Gln Lys Leu Tyr Arg
Ser Gly Asp Asn Trp Thr His Ser Cys Gln Gln Cys Arg Cys Leu Glu
Gly Glu Ala Asp Cys Trp Pro Leu Ala Cys Pro Ser Leu Gly Cys Glu
Tyr Thr Ala Met Phe Glu Gly Glu Cys Cys Pro Arg Cys Val Ser Asp
Pro Cys Leu Ala Gly Asn Ile Ala Tyr Asp Ile Arg Lys Thr Cys Leu
Asp Ser Phe Gly Val Ser Arg Leu Ser Gly Ala Val Trp Thr Met Ala
Gly Ser Pro Cys Thr Thr Cys Lys Cys Lys Asn Tly Arg Val Cys Cys
Ser Val Asp Leu Glu Cys Ile Glu Asn Asn
Felis catus NELL1 isoform l amino acid sequence (SEQ ID NO: 13)
Met Pro Arg Asp Val Ile Leu Val Val Trp Phe Cys Val Cys Thr Ala
Arg Thr Val Val Gly Phe Gly Thr Asp Pro Asp Leu Gln Val Asp Ile
Ile Ala Glu Leu Asp Leu Val Asn Thr Thr Ala Gly Val Thr Gln Val
Ser Gly Leu His Asn Ala Ser Lys Ala Tyr Leu Phe Gln Glu Thr Glu
Arg Glu Ile His Ala Ala Pro His Val Ser Glu Lys Leu Ile Gln Leu
Phe Arg Asn Lys Ser Glu Phe Ser Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Ile Ser Ala Ser His
Leu Leu Leu His Val Asp Cys Asn Arg Ile Tyr Glu Arg Val Ile Asp
Pro Pro Glu Thr Asn Leu Pro Pro Gly Ser Asn Val Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Asn Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Asp Ala Cys
Pro Pro Leu Asn Cys Ser Glu Lys Asp His Ile Leu Pro Glu Asn Gln
Cys Cys Ser Val Cys Arg Gly His Asn Phe Cys Ala Glu Gly Pro Thr
Cys Gly Glu Asn Ser Glu Cys Lys Asn Trp Asn Thr Lys Ala Thr Cys
Glu Cys Lys Asn Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Gln Glu Gly Cys Arg Tyr Gly Gly Thr
Cys Val Ser Pro Asn Lys Cys Val Cys Pro Ser Gly Phe Thr Gly Ser
His Cys Glu Lys Asp Ile Asp Glu Cys Thr Glu Gly Ile Ile Glu Cys
His Asn His Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Ile Asp Ile Asp Glu Cys Ala Leu Arg Thr His Thr Cys Trp
Asn Asp Ser Ala Cys Ile Asn Leu Ala Gly Gly Phe Asp Cys Leu Cys
Pro Ser Gly Pro Ser Cys Ser Gly Asp Cys Pro His Glu Gly Gly Leu
Lys Arg Asn Gly Gln Val Trp Thr Leu Lys Glu Asp Arg Cys Ser Val
Cys Ser Cys Lys Asp Gly Lys Ile Phe Cys Arg Arg Thr Ala Cys Asp
Cys Gln Asn Pro Ser Val Asp Leu Phe Cys Cys Pro Glu Cys Asp Thr
Arg Val Thr Ser Gln Cvs Leu Asp Gln Asn Gly His Lys Leu Tyr Arg
Ser Gly Asp Asn Trp Thr His Ser Cys Gln Gln Cys Arg Cys Leu Glu
Gly Glu Val Asp Cys Trp Pro Leu thr Cys Pro Asn Leu Ser Cys Glu
Tyr Thr Ala Met Leu Glu Gly Glu Cys Cys Pro Arg Cys Val Ser Asp
Pro Cys Leu Ala Asp Asn Ile Ala Tyr Asp Ile Arg Lys Thr Cys Leu
Asp Ser Tyr Gly Ile Ser Arg Leu Ser Gly Ala Val Trp Thr Met Ala
Gly Ser Pro Cys Thr Thr Cys Lys Cys Lys Asn Gly Ser Val Cys Cys
Ser Val Asp Leu Glu Cys Leu His Asn Asn
Felis catus NELL1 isoform 2 amino acid sequence (SEQ ID NO: 14)
Met Pro Arg Asp Val Ile Leu Val Val Trp Phe Cys Val Cys Thr Ala
Arg Thr Val Val Gly Phe Gly Thr Asp Pro Asp Leu Gln Val Asp Ile
Ile Ala Glu Leu Asp Leu Val Asn Thr Thr Ala Gly Val Thr Gln Val
Ser Gly Leu His Asn Ala Ser Lys Ala Tyr Leu Phe Gln Glu Thr Glu
Arg Glu Ile His Ala Ala Pro His Val Ser Glu Lys Leu Ile Gln Leu
Phe Arg Asn Lys Ser Glu Phe Ser Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Ile Ser Ala Ser His
Leu Leu Leu His Val Asp Cys Asn Arg Ile Tyr Glu Arg Val Ile Asp
Pro Pro Glu Thr Asn Leu Pro Pro Gly Ser Asn Val Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Asn Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Fro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Asp Ala Cys
Pro Pro Leu Asn Cys Ser Glu Lys Asp His Ile Leu Pro Glu Asn Gln
Cys Cys Ser Val Cys Arg Gly His Asn Phe Cys Ala Glu Gly Pro Thr
Cys Gly Glu Asn Ser Glu Cys Lys Asn Trp Asn Thr Lys Ala Thr Cys
Glu Cys Lys Asn Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Gln Glu Gly Cys Arg Tyr Gly Gly Thr
Cys Val Ser Pro Asn Lys Cys Val Cys Pro Ser Gly Phe Thr Gly Ser
His Cys Glu Lys Asp Ile Asp Glu Cys Ala Leu Arg Thr His Thr Cys
Trp Asn Asp Ser Ala Cys Ile Asn Leu Ala Gly Gly Phe Asp Cys Leu
Cys Pro Ser Gly Pro Ser Cys Ser Gly Asp Cys Pro His Glu Gly Gly
Leu Lys Arg Asn Gly Gln Val Trp Thr Leu Lys Glu Asp Arg Cys Ser
Val Cys Ser Cys Lys Asp Gly Lys Ile Phe Cys Arg Arg Thr Ala Cys
Asp Cys Gln Asn Pro Ser Val Asp Leu Phe Cys Cys Pro Glu Cys Asp
Thr Arg Val Thr Ser Gln Cys Leu Asp Gln Asn Gly His Lys Leu Tyr
Arg Ser Gly Asp Asn Trp Thr His Ser Cys Gln Gln Cys Arg Cys Leu
Glu Gly Glu Val Asp Cys Trp Pro Leu Thr Cys Pro Asn Leu Ser Cys
Glu Tyr Thr Ala Met Leu Glu Gly Glu Cys Cys Pro Arg Cys Val Ser
Asp Pro Cys Leu Ala Asp Asn Ile Ala Tyr Asp Ile Arg Lys Thr Cys
Leu Asp Ser Tyr Gly Ile Ser Arg Leu Ser Gly Ala Val Trp Thr Met
Ala Gly Ser Pro Cys Thr Thr Cys Lys Cys Lys Asn Gly Ser Val Cys
Cys Ser Val Asp Leu Glu Cys Leu His Asn Asn
Felis catus NELL1 isoform 1 amino acid sequence (SEQ ID NO: 13)
Met Pro Arg Asp Val Ile Leu Val Val Trp Phe Cys Val Cys Thr Ala
Arg Thr Val Val Gly Phe Gly Thr Asp Pro Asp Leu Gln Val Asp Ile
Ile Ala Glu Leu Asp Leu Val Asn Thr Thr Ala Gly Val Thr Gln Val
Ser Gly Leu His Asn Ala Ser Lys Ala Tyr Leu Phe Gln Glu Thr Glu
Arg Glu Ile His Ala Ala Pro His Val Ser Glu Lys Leu Ile Gln Leu
Phe Arg Asn Lys Ser Glu Phe Ser Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Ile Ser Ala Ser His
Leu Leu Leu His Val Asp Cys Asn Arg Ile Tyr Glu Arg Val Ile Asp
Pro Pro Glu Thr Asn Leu Pro Pro Gly Ser Asn Val Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Asn Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Asp Ala Cys
Pro Pro Leu Asn Cys Ser Glu Lys Asp His Ile Leu Pro Glu Asn Gln
Cys Cys Ser Val Cys Arg Gly His Asn Phe Cys Ala Glu Gly Pro Thr
Cys Gly Glu Asn Ser Glu Cys Lys Asn Trp Asn Thr Lys Ala Thr Cys
Glu Cys Lys Asn Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Gln Glu Gly Cys Arg Tyr Gly Gly Thr
Cys Val Ser Pro Asn Lys Cys Val Cys Pro Ser Gly Phe Thr Gly Ser
His Cys Glu Lys Asp Ile Asp Glu Cys Thr Glu Gly Ile Ile Glu Cys
His Asn His Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Ile Asp Ile Asp Glu Cys Ala Leu Arg Thr His Thr Cys Trp
Asn Asp Ser Ala Cys Ile Asn Leu Ala Gly Gly Phe Asp Cys Leu Cys
Pro Ser Gly Pro Ser Cys Ser Gly Asp Cys Pro His Glu Gly Gly Leu
Lys Arg Asn Gly Gln Val Trp Thr Leu Lys Glu Asp Arg Cys Ser Val
Cys Ser Cys Lys Asp Gly Lys Ile Phe Cys Arg Arg Thr Ala Cys Asp
Cys Gln Asn Pro Ser Val Asp Leu Phe Cys Cys Pro Glu Cys Asp Thr
Arg Val Thr Ser Gln Cys Leu Asp Gln Asn Gly His Lys Leu Tyr Arg
Ser Gly Asp Asn Trp Thr His Ser Cys Gln Gln Cys Arg Cys Leu Glu
Gly Glu Val Asp Cys Trp Pro Leu Thr Cys Pro Asn Leu Ser Cys Glu
Tyr Thr Ala Met Leu Glu Gly Glu Cys Cys Pro Arg Cys Val Ser Asp
Pro Cys Leu Ala Asp Asn Ile Ala Tyr Asp Ile Arg Lys Thr Cys Leu
Asp Ser Tyr Gly Ile Ser Arg Leu Ser Gly Ala Val Trp Thr Met Ala
Gly Ser Pro Cys Thr Thr Cys Lys Cys Lys Asn Gly Ser Val Cys Cys
Ser Val Asp Leu Glu Cys Leu His Asn Asn
Felis catus NELL1 isoform 2 amino acid sequence (SEQ ID NO: 14)
Met Pro Arg Asp Val Ile Leu Val Val Trp Phe Cys Val Cys Thr Ala
Arg Thr Val Val Gly Phe Gly Thr Asp Pro Asp Leu Gln Val Asp Ile
Ile Ala Glu Leu Asp Leu Val Asn Thr Thr Ala Gly Val Thr Gln Val
Ser Gly Leu His Asn Ala Ser Lys Ala Tyr Leu Phe Gln Glu Thr Glu
Arg Glu Ile His Ala Ala Pro His Val Ser Glu Lys Leu Ile Gln Leu
Phe Arg Asn Lys Ser Glu Phe Ser Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Ile Ser Ala Ser His
Leu Leu Leu His Val Asp Cys Asn Arg Ile Tyr Glu Arg Val Ile Asp
Pro Pro Glu Thr Asn Leu Pro Pro Gly Ser Asn Val Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Asn Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Asp Ala Cys
Pro Pro Leu Asn Cys Ser Glu Lys Asp His Ile Leu Pro Glu Asn Gln
Cys Cys Ser Val Cys Arg Gly His Asn Phe Cys Ala Glu Gly Pro Thr
Cys Gly Glu Asn Ser Glu Cys Lys Asn Trp Asn Thr Lys Ala Thr Cys
Clu Cys Lys Asn Cly Tyr Ile Ser Val Cln Cly Asp Sor Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Gln Glu Gly Cys Arg Tyr Gly Gly Thr
Cys Val Ser Pro Asn Lys Cys Val Cys Pro Ser Gly Phe Thr Gly Ser
His Cys Glu Lys Asp Ile Asp Glu Cys Ala Leu Arg Thr His Thr Cys
Trp Asn Asp Ser Ala Cys Ile Asn Leu Ala Gly Gly Phe Asp Cys Leu
Cys Pro Ser Gly Pro Ser Cys Ser Gly Asp Cys Pro His Glu Gly Gly
Leu Lys Arg Asn Gly Gln Val Trp Thr Leu Lys Glu Asp Arg Cys Ser
Val Cys Ser Cys Lys Asp Gly Lys Ile Phe Cys Arg Arg Thr Ala Cys
Asp Cys Gln Asn Pro Ser Val Asp Leu Phe Cys Cys Pro Glu Cys Asp
Thr Arg Val Thr Ser Gln Cys Leu Asp Gln Asn Gly His Lys Leu Tyr
Arg Ser Gly Asp Asn Trp Thr His Ser Cys Gln Gln Cys Arg Cys Leu
Glu Gly Glu Val Asp Cys Trp Pro Leu Thr Cys Pro Asn Leu Ser Cys
Glu Tyr Thr Ala Met Leu Glu Gly Glu Cys Cys Pro Arg Cys Val Ser
Asp Pro Cys Leu Ala Asp Asn Ile Ala Tyr Asp Ile Arg Lys Thr Cys
Leu Asp Ser Tyr Gly Ile Ser Arg Leu Ser Gly Ala Val Trp Thr Met
Ala Gly Ser Pro Cys Thr Thr Cys Lys Cys Lys Asn Gly Ser Val Cys
Cys Ser Val Asp Leu Glu Cys Leu His Asn Asn
Canis lupis familiaris NELL1 amino acid sequence (SEP ID NO: 15)
Met Thr Ser Thr Ser Phe Leu Leu Trp Leu Gly Cys Val His Asn Thr
Lys Phe Pro Phe Pro Leu Val Leu Val Thr Arg Ala Ile Val Val Val
Val Val Glu Val Val Gly Val Gly Ser Pro Gly Val Arg Ile Arg Ser
Thr Gly Cys Asp Ile Leu Leu Leu Tyr Glu Val Leu Glu His Leu Leu
Gly Ile Arg Phe Leu Cys Val Asp Gln Gly Glu Asn Ser Cys His His
Gly Gln Cys Ala Cys Arg Leu Gln Val Ile Val Pro Lys Ala Leu Met
Ser Val Phe Glu Ala Lys Thr Ala Val Cys Phe Phe Pro Val Val Gly
Phe Gly Thr Asp Pro Asp Leu Gln Met Asp Ile Ile Thr Glu Leu Asp
Leu Val Asn Ile Ser Leu Gly Val Thr Gln Val Ser Gly Leu His Asn
Ala Ser Lys Ala Tyr Val Phe Gln Asp Thr Ala Arg Glu Ile His Ala
Ala Pro His Val Ser Glu Lys Leu Ile Gln Leu Phe Arg Asn Lys Ser
Asp Phe Thr Phe Leu Ala Thr Val Gln Gln Lys Pro Ser Thr Ser Gly
Val Ile Leu Ser Ile Arg Glu Leu Glu His Ser Tyr Phe Glu Leu Glu
Ser Ser Gly Leu Arg Asp Glu Ile Arg Tyr His Tyr Met His Asn Gly
Lys Pro Arg Thr Glu Ala Leu Pro Tyr Arg Leu Ala Asp Gly Gln Trp
His Lys Val Ala Leu Ser Val Ser Ala Ser His Leu Leu Leu His Ile
Asp Cys Asn Arg Ile Tyr Glu Arg Val Ile Asp Pro Pro Glu Thr Asn
Leu Pro Pro Gly Ser Asn Leu Trp Leu Gly Gln Arg Asn Gln Lys His
Gly Phe Phe Lys Gly Ile Ile Gln Asp Gly Lys Ile Ile Phe Met Pro
Asn Gly Tyr Ile Thr Gln Cys Pro Asn Leu Asn Arg Thr Cys Pro Thr
Cys Ser Asp Phe Leu Ser Leu Val Gln Gly Ile Met Asp Leu Gln Glu
Leu Leu Ala Lys Met Thr Ala Lys Leu Asn Tyr Ala Glu Thr Arg Leu
Ser Gln Leu Glu Asn Cys His Cys Glu Lys Thr Cys Gln Val Ser Gly
Leu Leu Tyr Arg Asp Gln Asp Ser Trp Val Asp Gly Asp His Cys Arg
Asn Cys Thr Cys Lys Gly Gly Ala Val Glu Cys Arg Arg Met Ser Cys
Pro Pro Leu Asn Cys Ser Pro Asp Ser Leu Pro Val His Ile Ala Gly
Gln Cys Cys Lys Val Cys Arg Pro Lys Cys Ile Tyr Gly Gly Arg Val
Leu Ala Glu Gly Gln Arg Ile Leu Thr Lys Ser Cys Arg Glu Cys Arg
Gly Gly Val Leu Val Lys Ile Thr Asp Ala Cys Pro Pro Leu Asn Cys
Ser Glu Lys Asp His Ile Leu Pro Glu Asn Gln Cys Cys Ser Val Cys
Arg Gly His Asn Phe Cys Ala Glu Gly Pro Lys Cys Gly Glu Asn Ser
Glu Cys Lys Asn Trp Asn Thr Lys Ala Thr Cys Glu Cys Lys Asn Gly
Tyr Ile Ser Val Gln Gly Asp Ser Ala Tyr Cys Glu Asp Ile Asp Glu
Cys Ala Ala Lys Met His Tyr Cys His Ala Asn Thr Val Cys Val Asn
Leu Pro Gly Leu Tyr Arg Cys Asp Cys Val Pro Gly Tyr Ile Arg Val
Asp Asp Phe Ser Cys Thr Glu His Asp Glu Cys Gly Ser Gly Gln His
Asn Cys Asp Glu Asn Ala Ile Cys Thr Asn Thr Val Arg Gly His Ser
Cys Thr Cys Lys Pro Gly Tyr Val Gly Asn Gly Thr Ile Cys Arg Ala
Phe Cys Gln Glu Gly Cys Arg Tyr Gly Gly Ser Cys Val Ser Pro Asn
Lys Cys Val Cys Pro Ser Gly Phe Thr Gly Ser His Cys Glu Lys Asp
Ile Asp Glu Cys Thr Glu Gly Ile Ile Glu Cys His Asn His Ser Arg
Cys Val Asn Leu Pro Gly Trp Tyr His Cys Glu Cys Arg Ser Gly Phe
His Asp Asp Gly Thr Tyr Ser Leu Ser Gly Glu Ser Cys Ile Asp Ile
Asp Glu Cys Ala Leu Arg Thr His Thr Cys Trp Asn Asp Ser Ala Cys
Ile Asn Leu Ala Gly Gly Phe Asp Cys Leu Cys Pro Ser Gly Pro Ser
Cys Ser Gly Asp Cys Pro His Glu Gly Gly Leu Lys Arg Asn Gly Gln
Val Trp Thr Leu Lys Glu Asp Arg Cys Ser Val Cys Ser Cys Lys Asp
Gly Lys Ile Leu Cys Arg Arg Thr Ala Cys Asp Cys Gln Asn Pro Ser
Val Asp Leu Phe Cys Cys Pro Glu Cys Asp Thr Arg Val Thr Ser Gln
Cys Leu Asp Gln Asn Gly His Lys Leu Tyr Arg Ser Gly Asp Asn Trp
Thr His Ser Cys Gln Gln Cys Arg Cys Leu Glu Gly Glu Val Asp Cys
Trp Pro Leu Thr Cys Pro Asn Leu Ser Cys Glu Tyr Thr Ala Ile Leu
Glu Gly Glu Cys Cys Pro Arg Cys Val Ser Asp Pro Cys Leu Ala Asp
Asn Ile Ala Tyr Asp Ile Arg Lys Thr Cys Leu Asp Ser Tyr Gly Ile
Ser Arg Leu Ser Gly Ser Val Trp Thr Met Ala Gly Ser Pro Cys Thr
Thr Cys Lys Cys Lys Asn Gly Ser Val Cys Cys Ser Val Asp Leu Glu
Cys Leu His Asn Asn
Ovis aries NELL1 amino acid sequence (SEQ ID NO: 16)
Met Pro Arg Gly Val Ile Leu Val Val Cys Phe Cys Val Cys Ala Ala
Arg Thr Val Val Gly Phe Gly Met Asp Pro Asp Leu Gln Leu Asp Ile
Ile Thr Glu Leu Asp Leu Val Asn Thr Thr Leu Gly Val Thr Gln Val
Ser Gly Leu His Asn Thr Ser Lys Ala Phe Leu Phe Gln Asp Ala Glu
Arg Glu Ile His Ala Ala Pro His Val Ser Glu Lys Leu Ile Gln Leu
Phe Arg Asn Lys Ser Glu Phe Thr Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Met His Ser Gly Arg Pro Arg Thr Glu Ala Leu Pro Tyr Arg Leu
Ala Asp Gly Gln Trp His Arg Val Ala Leu Ser Val Ser Ala Ser His
Leu Leu Leu His Ile Asp Cys Asn Arg Ile Tyr Glu Arg Val Ile Asp
Pro Pro Glu Thr Asn Leu Pro Pro Gly Ser Asn Leu Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Ser Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Ser Lys Asn
Cys Gln Glu Cys Arg Gly Gly Val Leu Val Lys Ile Thr Glu Ala Cys
Pro Leu Leu Asn Cys Ser Glu Lys Asp His Ile Leu Pro Glu Asn Gln
Cys Cys Ser Val Cys Arg Gly His Asn Phe Cys Ala Glu Gly Pro Lys
Cys Gly Glu Asn Ser Glu Cys Lys Asn Trp Asn Thr Lys Ala Thr Cys
Glu Cys Lys Asn Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Asp Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Glu Glu Gly Cys Arg Tyr Gly Gly Thr
Cys Met Ala Pro Asn Lys Cys Val Cys Pro Ser Gly Phe Thr Gly Ser
His Cys Glu Lys Asp Ile Asp Glu Cys Ala Glu Gly Ile Ile Glu Cys
His Ser His Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Val Asp Ile Asp Glu Cys Ala Leu Arg Thr His Thr Cys Trp
Asn Asp Ser Ala Cys Ile Asn Leu Ala Gly Gly Phe Asp Cys Leu Cys
Pro Ser Gly Pro Ser Cys Ser Gly Asp Cys Pro His Glu Gly Gly Leu
Lys Arg Asn Gly Gln Val Trp Thr Leu Lys Glu Asp Arg Cys Ser Val
Cys Ser Cys Lys Asp Gly Lys Ile Phe Cys Arg Arg Thr Ala Cys Asp
Cys Gln Asn Pro Ser Val Asp Leu Phe Cys Cys Pro Glu Cys Asp Thr
Arg Val Thr Ser Gln Cys Leu Asp Gln Asn Gly Asn Lys Leu Tyr Arg
Ser Gly Asp Asn Trp Thr His Ser Cys Gln Gln Cys Arg Cys Leu Glu
Gly Glu Val Asp Cys Trp Pro Leu Thr Cys Pro Ser Leu Ser Cys Glu
Tyr Thr Thr Ile Leu Glu Glu Glu Cys Cys Pro Arg Cys Val Ser Asp
Pro Cys Leu Ala Asp Asn Ile Ala Tyr Asp Ile Arg Lys Thr Cys Leu
Asp Ser Tyr Gly Leu Ser Arg Leu Ser Gly Ser Val Trp Thr Met Ala
Gly Ser Pro Cys Thr Thr Cys Lys Cys Lys Asn Gly Ser Val cys Cys
Ser Val Asp Leu Glu Cys Leu His Asn Asn
Homo sapiens NELL1 fragment amino acid sequence (SEQ ID NO: 17)
Phe Gly Met Asp Pro Asp Leu Gln Met Asp Ile
Val Thr Glu Leu Asp Leu Val Asn Thr Thr Leu Gly Val Ala Gln Val
Ser Gly Met His Asn Ala Ser Lys Ala Phe Leu Phe Gln Asp Ile Glu
Arg Glu Ile His Ala Ala Pro His Val Ser Glu Lys Leu Ile Gln Leu
Phe Arg Asn Lys Ser Glu Phe Thr Ile Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Val Ser Ala Ser His
Leu Leu Leu His Val Asp Cys Asn Arg Ile Tyr Glu Arg Val Ile Asp
Pro Pro Asp Thr Asn Leu Pro Pro Gly Ile Asn Leu Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Tro Asn Leu Asn
His Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Ser Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Glu Met Cys
Pro Pro Leu Asn Cys Ser Glu Lys Asp His Ile Leu Pro Glu Asn Gln
Cys Cys Arg Val Cys Arg Gly His Asn Phe Cys Ala Glu Gly Pro Lys
Cys Gly Glu Asn Ser Glu Cys Lys Asn Trp Asn Thr Lys Ala Thr Cys
Glu Cys Lys Ser Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Glu Glu Gly Cys Arg Tyr Gly Gly Thr
Cys Val Ala Pro Asn Lys Cys Val Cys Pro Ser Gly Phe Thr Gly Ser
His Cys Glu Lys Asp Ile Asp Glu Cys Ser Glu Gly Ile Ile Glu Cys
His Asn His Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Ile Asp Ile Asp Glu Cys Ala Leu Arg Thr His Thr Cys Trp
Asn Asp Ser Ala Cys Ile Asn Leu Ala Gly Gly Phe Asp Cys Leu Cys
Pro Ser Gly Pro Ser Cys Ser
Equus caballus NELL1 fragment amino acid seauence (SEQ ID NO: 18)
Phe Gly Met Asp Pro Asp Leu Gln Met Asp Ile Ile Thr Glu
Leu Asp Leu Val Asn Thr Thr Leu Gly Val Thr Gln Val Ser Gly Leu
His Asn Ala Ser Lys Ala Phe Leu Phe Gln Asp Val Glu Arg Glu Ile
His Ala Ala Pro His Val Ser Glu Lys Leu Ile Gln Leu Phe Arg Asn
Lys Ser Glu Phe Thr Phe Leu Ala Thr Val Gln Gln Lys Pro Ser Thr
Ser Gly Val Ile Leu Ser Ile Arg Glu Leu Glu Asn Ser Tyr Phe Glu
Leu Glu Ser Ser Gly Leu Arg Asp Glu Ile Arg Tyr His Tyr Thr His
Lys Gly Lys Pro Arg Thr Glu Ala Leu Pro Tyr Arg Met Ala Asp Gly
Arg Trp His Lys Val Ala Leu Ser Val Ser Ala Ser His Leu Leu Leu
His Ile Asp Cys Asn Arg Ile Tyr Glu Arg Val Ile Asp Thr Pro Glu
Thr Asn Leu Pro Pro Gly Ser Asn Leu Trp Leu Gly Gln Arg Asn Gln
Lys His Gly Leu Phe Lys Gly Ile Ile Gln Asp Gly Lys Ile Ile Phe
Met Pro Asn Gly Tyr Ile Thr Gln Cys Pro Asn Leu Asn Arg Thr Cys
Pro Thr Cys Ser Asp Phe Leu Ser Leu Val Gln Gly Ile Met Asp Leu
Gln Glu Leu Leu Ala Lys Met Thr Ala Lys Leu Asn Tyr Ala Glu Thr
Arg Leu Ser Gln Leu Glu Asn Cys His Cys Glu Lys Thr Cys Gln Val
Ser Gly Leu Leu Tyr Arg Asp Gln Asp Ser Trp Val Asp Gly Asp His
Cys Arg Asn Cys Thr Cys Lys Ser Gly Ala Val Glu Cys Arg Arg Met
Ser Cys Pro Pro Leu Asn Cys Ser Pro Asp Ser Leu Pro Val His Val
Ala Gly Gln Cys Cys Lys Val Cys Arg Pro Lys Cys Ile Tyr Gly Gly
Lys Val Leu Ala Glu Gly Gln Arg Ile Leu Thr Lys Ser Cys Arg Glu
Cys Arg Gly Gly Val Leu Val Lys Ile Thr Glu Ala Cys Pro Pro Leu
Asn Cys Ser Asp Lys Asp His Ile Leu Pro Glu Asn Gln Cys Cys Ser
Val Cys Arg Gly His Asn Phe Cys Ala Glu Gly Pro Lys Cys Gly Glu
Asn Ser Glu Cys Lys Asn Trp Asn Thr Lys Ala Thr Cys Glu Cys Lys
Asn Gly Tyr Ile Ser Val Gln Gly Asp Ser Ala Tyr Cys Glu Asp Ile
Asp Glu Cys Ala Ala Lys Met His Tyr Cys Arg Ala Asn Thr Val Cys
Val Asn Leu Pro Gly Leu Tyr Arg Cys Asp Cys Val Pro Gly Tyr Ile
Arg Val Asp Asp Phe Ser Cys Thr Glu His Asp Glu Cys Gly Ser Gly
Gln His Asn Cys Asp Glu Asn Ala Ile Cys Thr Asn Thr Val Gln Gly
His Ser Cys Thr Cys Lys Pro Gly Tyr Val Gly Asn Gly Thr Ser Cys
Arg Ala Phe Cys Glu Glu Gly Cys Arg Tyr Gly Gly Thr Cys Val Ala
Pro Asn Lys Cys Val Cys Pro Ser Gly Phe Thr Gly Ser His Cys Glu
Lys Asp Ile Asp Glu Cys Thr Glu Gly Ile Ile Glu Cys His Asn His
Ser Arg Cys Val Asn Leu Pro Gly Trp Tyr His Cys Glu Cys Arg Ser
Gly Phe His Asp Asp Gly Thr Tyr Ser Leu Ser Gly Glu Ser Cys Ile
Asp Ile Asp Glu Cys Ala Leu Arg Thr His Thr Cys Trp Asn Asp Ser
Ala Cys Ile Asn Leu Ala Gly Gly Phe Asp Cys Leu Cys Pro Ser Gly
Pro Ser Cys Ser
Bos taurus NELL1 amino acid sequence (SEQ ID NO: 19)
Met Ala Leu Cys Ser Phe Ser Val Val Gly Phe Gly Leu Asp Pro Asp
Leu Gln Leu Asp Ile Ile Thr Glu Leu Asp Leu Val Asn Thr Thr Leu
Gly Val Thr Gln Val Ser Gly Leu His Asn Thr Ser Lys Ala Phe Leu
Phe Gln Asp Ala Glu Arg Glu Ile His Ala Ala Pro His Val Ser Glu
Lys Leu Ile Gln Leu Phe Arg Asn Lys Ser Glu Phe Thr Phe Leu Ala
Thr Val Gln Gln Lys Pro Ser Thr Ser Gly Val Ile Leu Ser Ile Arg
Glu Leu Glu His Ser Tyr Phe Glu Leu Glu Ser Ser Gly Leu Arg Asp
Glu Ile Arg Tyr His Tyr Val His Ser Gly Arg Pro Arg Thr Glu Ala
Leu Pro Tyr Arg Leu Ala Asp Gly Gln Trp His Arg Val Ala Leu Ser
Val Ser Ala Ser His Leu Leu Leu His Ile Asp Cys Asn Arg Ile Tyr
Glu Arg Val Ile Asp Pro Pro Glu Thr Asn Leu Pro Pro Gly Ser Asn
Leu Trp Leu Gly Gln Arg Asn Gln Lys His Gly Leu Phe Lys Gly Ile
Ile Gln Asp Gly Lys Ile Ile Phe Met Pro Asn Gly Tyr Ile Thr Gln
Cys Pro Asn Leu Asn Arg Thr Cys Pro Thr Cys Ser Asp Phe Leu Ser
Leu Val Gln Gly Ile Met Asp Leu Gln Glu Leu Leu Ala Lys Met Thr
Ala Lys Leu Asn Tyr Ala Glu Thr Arg Leu Ser Gln Leu Glu Asn Cys
His Cys Glu Lys Thr Cys Gln Val Ser Gly Leu Leu Tyr Arg Asp Gln
Asp Ser Trp Val Asp Gly Asp His Cys Arg Asn Cys Thr Cys Lys Ser
Gly Ala Val Glu Cys Arg Arg Met Ser Cys Pro Pro Leu Asn Cys Ser
Pro Asp Ser Leu Pro Val His Ile Ala Gly Glu Cys Cys Lys Val Cys
Arg Pro Lys Cys Ile Tyr Gly Gly Lys Val Leu Ala Glu Gly Gln Arg
Ile Leu Ser Lys Ser Cys Gln Glu Cys Arg Gly Gly Val Leu Val Lys
Ile Thr Glu Ala Cys Pro Leu Leu Asn Cys Ser Glu Lys Asp His Ile
Leu Pro Glu Asn Gln Cys Cys Ser Val Cys Arg Gly His Asn Phe Cys
Ala Glu Gly Pro Lys Cys Gly Glu Asn Ser Glu Cys Lys Asn Trp Asn
Thr Lys Ala Thr Cys Glu Cys Lys Asn Gly Tyr Ile Ser Val Gln Gly
Asp Ser Ala Tyr Cys Glu Asp Ile Asp Glu Cys Ala Ala Lys Met His
Tyr Cys His Ala Asn Thr Val Cys Val Asn Leu Pro Gly Leu Tyr Arg
Cys Asp Cys Val Pro Gly Tyr Ile Arg Val Asp Asp Phe Ser Cys Thr
Glu His Asp Asp Cys Gly Ser Gly Gln His Asn Cys Asp Glu Asn Ala
Ile Cys Thr Asn Thr Val Gln Gly His Ser Cys Thr Cys Lys Pro Gly
Tyr Val Gly Asn Gly Thr Ile Cys Arg Gly Met Pro Glu Val Gly Fro
Pro Arg Ala Leu Leu Asn Ser Leu Asp Leu Gly Phe Leu Ser Phe Ser
Lys Glu Ala Leu Ala Val Gly Met Ile Thr Leu Glu Gly Asn Ile Val
Ala Lys Ser Phe Thr Asp Asp Glu Thr Leu Val Glu Arg Gly Arg Glu
Lys Val Ile Ala Leu Leu Phe Ser Trp Leu His Lys Glu Lys Leu Ser
Leu Glu Asn Leu Arg Asp Ile Tyr Cys Lys Ala Asn Ser Leu Val Gly
Leu Asp His Leu Pro Gln Arg
Homo sapiens interleukin-1 beta proprotein amino acid sequence (SEQ ID NO: 20)
Met Ala Glu Val Pro Glu Leu Ala Ser Glu Met Met Ala Tyr Tyr Ser
Gly Asn Glu Asp Asp Leu Phe Phe Glu Ala Asp Gly Pro Lys Gln Met
Lys Cys Ser Phe Gln Asp Leu Asp Leu Cys Pro Leu Asp Gly Gly Ile
Gln Leu Arg Ile Ser Asp His His Tyr Ser Lys Gly Phe Arg Gln Ala
Ala Ser Val Val Val Ala Met Asp Lys Leu Arg Lys Met Leu Val Fro
Cys Pro Gln Thr Phe Gln Glu Asn Asp Leu Ser Thr Phe Phe Pro Phe
Ile Phe Glu Glu Glu Pro Ile Phe Phe Asp Thr Trp Asp Asn Glu Ala
Tyr Val His Asp Ala Pro Val Arg Ser Leu Asn Cys Thr Leu Arg Asp
Ser Gln Gln Lys Ser Leu Val Met Ser Gly Pro Tyr Glu Leu Lys Ala
Leu His Leu Gln Gly Gln Asp Met Glu Gln Gln Val Val Phe Ser Met
Ser Phe Val Gln Gly Glu Glu Ser Asn Asp Lys Ile Pro Val Ala Leu
Gly Leu Lys Glu Lys Asn Leu Tyr Leu Ser Cys Val Leu Lys Asp Asp
Lys Pro Thr Leu Gln Leu Glu Ser Val Asp Pro Lys Asn Tyr Pro Lys
Lys Lys Met Glu Lys Arg Phe Val Phe Asn Lys Ile Glu Ile Asn Asn
Lys Leu Glu Phe Glu Ser Ala Gln Phe Pro Asn Trp Tyr Ile Ser Thr
Gln Asp Ile Thr Asp Phe Thr Met Gln Phe Val Ser Ser

### Examples

The present invention, thus generally described, will be understood more readily by reference to the following Examples, which are provided by way of illustration and are not intended to be limiting of the instant invention. The Examples are not intended to represent that the experiments below are all experiments performed.

### Example 1

### NELL1 NV1 Fragment Rescues Atrophy in In Vitro Model

The objective of this study was to test the effects of the human NELL1 NV1 fragment (SEQ ID NO: 17) on muscle atrophy in an in vitro model.

The study was conducted on pediatric myoblast cells with a contract research organization, CYTOO (Grenoble, France) using a robust muscle atrophy rescue assay developed by the company called MYOSCREEN™ (Young et al. 2018 SLAS Discov 23(8):790-806, doi: 10.1177/2472555218761102). The MYOSCREEN™ platform is a robust human in vitro model for muscle atrophy. This platform uses micropatterned myotubes that form aligned striated myofibers with physiologically and pharmacologically relevant features characteristic of mature skeletal muscle.

### Materials and Methods

Human primary myoblasts were amplified following CYTOO Standard Operation Procedures wherein the cells were allowed to proliferate in a flask in a growth medium, and were passaged in order to obtain enough cells to run the assay. The patient included in this study was AA179, cells from an 8-year-old male.

Cells were seeded on Day 0 in two MYOSCREEN™ plates (one for each donor) in a growth medium. On Day 1, growth medium was removed from the two plates and substituted with a differentiation medium to induce myoblast fusion. On Day 2, the differentiation medium was changed and the treatments were added. A stock solution of NV1 (in PBS) was added at five concentrations (0.2 μg/ml, 0.4 μg/ml, 0.6 μg/ml, 0.8 μg/ml, and 1 μg/ml) with 3-well replicates for the lower NV1 concentration and 4 well-replicates for the other concentrations, and insulin-like growth factor-1 (IGF-1) at 100 ng/ml. After a two-hour incubation, samples were treated with 20 ng/ml IL-1β.

On Day 6, the cells were prepared for immunostaining by fixing with formalin. The fixed cells were processed for immunostaining by staining nuclei with Hoechst dye and myotubes were stained with a Troponin T specific antibody. Images were acquired on the Operetta High Content Imaging System (Perkin Elmer) using a 10× objective in two fluorescent channels, nuclei (Hoechst) and Troponin T. Myotubes were characterized for parameters related to viability, differentiation and morphology, specifically fusion index, myotube area, and number of nuclei per well. Image processing and analysis were performed with dedicated algorithms developed on the Acapella High Content Imaging Software (Perkin Elmer) by CYTOO. Eleven fields per well were acquired.

First, segmentation of myotubes and nuclei are performed using, respectively, the Troponin T staining intensity and the Hoechst staining. One to two myotubes per micropattern were usually identified (a myotube is a troponin T staining area that includes at least two nuclei). The threshold of segmentation was set-up in order to avoid detecting the background noise and to eliminate aberrant small myotube structures. At the end of this first step, specific readouts were calculated in the whole well including the total nuclei count per well, the total myotube area per well, and the fusion index (percentage of nuclei included in troponin T staining). Usually around 50 to 60 myotubes were detected per well in a control condition.

Then, an image clean-up step was performed on the Troponin T images in order to remove myotubes that touch the border of the image. The final valid myotubes were used to extract myotube morphology parameters including the myotube area and width and the number of nuclei per myotube.

### Results

1. Quality Control

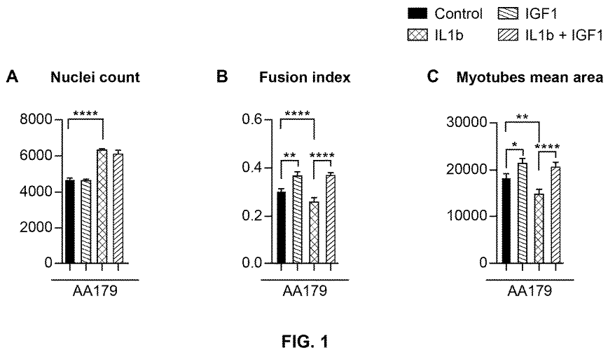

Muscle cells were differentiated for five days, including four days of treatment. At the end of the experiment, myotubes were stained with an antibody against Troponin T, and image analysis was performed to characterize differentiation. Results are shown in FIG. 1.

IGF-1, a positive control for inducing hypertrophy, significantly increased the myotube fusion index (+21%) and the myotube mean area (+17%).

Atrophy was induced with IL-1β at 20 ng/ml. IL-1β increased the nuclei count, and significantly decreased the myotube fusion index (−15%) as well as myotube mean area (−18%). IGF-1 significantly rescued the atrophy induced by IL-1β, increasing the fusion index by +240%, and the myotube mean area by +175%, compared to the IL-1β results.

2. NV1 Results

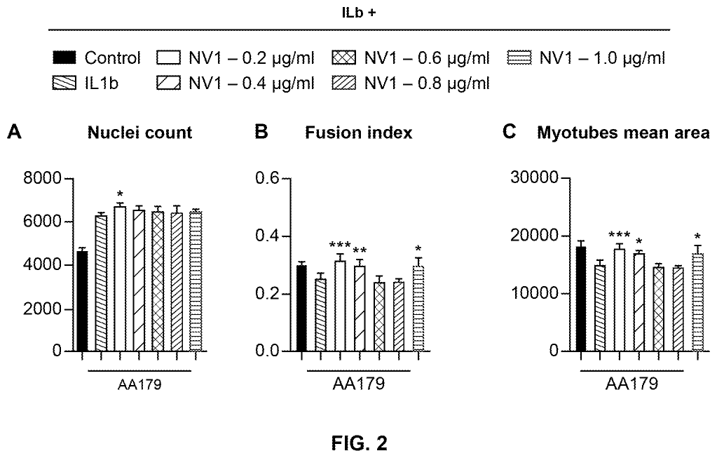

NV1 was tested in the MYOSCREEN™ atrophy rescue assay at five concentrations ranging from 0.2 μg/ml to 1 μg/ml. As shown in FIG. 2, NV1 treatment had a significant rescue effect on the pediatric myotubes (AA179) at three of the tested doses, both on the fusion index parameter and the myotube mean area parameter. The 0.2 μg/ml dose induced a complete rescue of the fusion index parameter (+133%) and rescued by 86% the myotube mean area parameter. The 0.4 μg/ml and 1 μg/ml concentrations rescued the fusion index parameter by 98% and 99% respectively, as well as the myotube mean area parameter by 60% and 62%, respectively. Overall, NV1 induced a positive effect on the myotube differentiation and size in the pediatric myotubes in the IL-1β atrophy rescue assay.

### CONCLUSIONS

In this study, myotubes responded by hypertrophy induced by IGF-1 and by atrophy induced by IL-1β. In addition, an atrophy rescue effect was measured when combining IGF-1 with IL-1β, validating the atrophy rescue assay.

The NELL1 NV1 fragment was tested at concentrations ranging from 0.2 μg/ml to 1 μg/ml in IL-1β atrophied conditions to measure rescue effects, using pediatric myotubes. No negative effects on myotube health were observed in the tested conditions.

NV1 showed rescue capacity in the IL-1β atrophy rescue assay in pediatric myotubes, inducing an almost complete rescue when used at a 0.2 μg/ml concentration. Thus, the NELL1 NV1 fragment was able to completely rescue pediatric muscle tissue from atrophy induced by IL-1β. NV1 was able to restore both myotube differentiation and area to normal levels at multiple concentrations. This result was surprising because a similar result was not seen in preliminary studies in adult myotubes (from a 21-year-old male) nor could the atrophy induced by TNF-α be rescued by any of the tested concentrations of NV1 in preliminary studies (data not shown).

While not intending to be bound to any particular theory, these data may reflect an increased sensitivity to inflammation-induced atrophy in pediatric patients.

### Example 2

### Effects of NELL1 on a Microgravity Model of Muscle Atrophy

NELL1 treatment for MA is tested using a microgravity model in the International Space Station (ISS; Lalani R et al. 2000 Journal of Endocrinology 167:417-428; Cadena S M et al. 2019 Scientific Reports 9:9397). Rodents at the ISS experience muscle atrophy and the candidate drug (NV1) is administered. The treated mice are then examined for hallmarks of muscle formation to test the drug's effects.

More specifically, NV1 is tested as follows. Forty age-matched 12-week old female mice, strain C57Bl/6, are randomly assigned into four groups with 10 mice per group (A, B, C, and D). The treatment groups are: A—Control, 1-month atrophy, B—NV1-treated, 1-month atrophy, C—Control, 2-month atrophy, and D—NV1-treated, 2-month atrophy. Untreated mice are launched to space and transferred to the rodent habitats at the ISS and acclimatized for 24 hours. Functional assessments and injections (every 10 days) are conducted. Mass and grip strength measurements are made. Either phosphate buffered saline (PBS control) or NV1 is injected subcutaneously (s.c.) to the respective group of mice. A subcutaneous (s.c.) route is a physiologically relevant route for NV1 human drug development. NV1 is administered s.c. at a dose of 5 mg/kg weight per injection that corresponds to 28 micrograms/mouse, assuming an average weight of 22 grams per mouse. Pre-loaded syringes with 28 micrograms NV1/0.10 ml (10 injections per syringe or 4-5 syringes for 40 mice) are prepared. Injections are administered at 10-day intervals on Days 4-5, 14-15, and 24-25 for all groups. Additional injections are given to Groups C and D at Days 34-35, 44-45 and 54-55.

After a month, the 20 mice (10 control, 10 NV1-treated) in Groups A and B are euthanized by exsanguination and cervical dislocation after blood is collected via cardiac puncture. Hearts and both leg (gastrocnemius) muscles are collected via punch biopsies. The right leg muscle is preserved for histology and the left leg muscle is preserved in RNALater for molecular analysis. Whole blood is separated by centrifugation, frozen and stored at −80° C. or colder. Samples are returned to earth for further analysis. After the second month, Groups C and D are euthanized and processed as described for groups A and B. This second set is returned to earth on a later flight.

Skeletal and heart tissues are processed for gene expression assays on selected MA markers to evaluate if NV1 treated microgravity-induced atrophy. The levels of molecular markers such as NF-κB, IL-1β, IL6, IL8, myostatin, atrogin-1/MAFbx, and MMP1 are measured. Luminex-based Quantigene assay or real time qPCR is used to quantify the target genes and two housekeeping control genes. Two histological parameters which are hallmarks of muscle structure, growth and differentiation (fusion index and myotube fiber area) are examined. Invitrogen's EVOS FL Auto Cell Imaging System is utilized to quantify immunofluorescently-stained myotubes. An ELISA or Luminex-based multiplex assay is used to measure selected inflammation markers in serum or plasma collected from mouse whole blood.

### Example 3

### Effects of NELL1 on a Cancer Cachexia Model

Several mouse models have been generated to test the efficacy of new drugs for MA to treat cancer cachexia. Typically, these models are prepared by injecting aggressive cancer cells into the animal and as the cancer becomes established and cachexia is manifested, the therapeutic is administered to the animals and compared to untreated controls. Examples of these models are xenograft Ion-26 (C26) and Lewis lung carcinoma (LLC) rodent models (Holecek M 2012 International Journal of Experimental Pathology 93:157-171; Romanick M and Brown-Berg H M 2013 Biochim Biophys Acta 1832(9):1410-1420). A more recent model is a genetically engineered mouse, the KPP mouse, which models pancreatic ductal adenocarcinoma (Talbert E E et al. 2019 Cell Reports 28:1612-1622). Using any of these models, mice with cancer cachexia are divided into two groups of 6-10 mice: 10 control and 10 treated. NV1 is injected in concentrations of 2-10 mg/kg and repeated after seven days (or weekly injections) at the onset of cachexia. After 30 days, the weight, muscle histology and function (contractility, strength and fatigue resistance) are evaluated.

Additional animal models (disuse and paralysis, starvation and anorexia models) are also used with the same study design described above (Holecek M 2012 Int. J. Exp. Path. 93:157-171; Mequinon M et al 2015 Frontiers in Endocrinology 6(68) doi:10.3389/fendo.2015.00068; Romanick M and Brown-Borg H M 2013 Biochim Biophys Acta 1832(9) : 1410-1420. doi:10,1016/j.bbadis.2013.03.011).

Those skilled in the art will further appreciate that the present invention may be embodied in other specific forms without departing from the spirit or central attributes thereof. In that the foregoing description of the present invention discloses exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. A method of treating muscle atrophy in a pediatric subject in need thereof, said method comprising administering an effective amount of a NELL1 polypeptide, or a nucleic acid molecule encoding the same.

2. The method of claim 1, wherein said pediatric subject has chronic systemic inflammation.

3. The method of embodiment 2, wherein said chronic systemic inflammation is due to a viral infection.

4. The method of claim 3, wherein said viral infection is by a coronavirus.

5. The method of claim 4, wherein said coronavirus is SARS-CoV-2.

6. The method of any one of claims 1-5, wherein said pediatric subject has increased circulating levels of IL-1β when compared to a control subject.

7. The method of any one of claims 1-6 wherein said pediatric subject has a genetic predisposition for increased circulating levels of IL-1β when compared to a control subject.

8. The method of any one of claims 1-7, wherein said pediatric subject has increased circulating levels of one or more of interleukin-8 (IL-8), nuclear factor kappa-light chain-enhancer of activated B cells (NF-κB), and matrix metalloproteinase 1 (MMP1) when compared to a control subject.

9. A method of treating muscle atrophy in a subject with chronic systemic inflammation, said method comprising administering an effective amount of a NELL1 polypeptide, or a nucleic acid molecule encoding the same.

10. The method of claim 9, wherein said subject is a pediatric subject.

11. The method of claim 9 or 10, wherein said subject has a cancer.

12. The method of claim 11, wherein said cancer is a stage III or IV cancer.

13. The method of claim 11 or 12, wherein said cancer is a pancreatic or gastric cancer.

14. The method of any one of claims 10-13, wherein said subject has increased circulating levels of IL-1β when compared to a control subject.

15. The method of any one of claims 9-14, wherein said subject has a genetic predisposition for increased circulating levels of IL-1β when compared to a control subject.

16. The method of any one of claims 9-15, wherein said subject has increased circulating levels of one or more of IL-8, NF-κB, and MMP1 when compared to a control subject.

17. The method of claim 9, wherein said chronic systemic inflammation is due to a viral infection.

18. The method of claim 17, wherein said viral infection is by a coronavirus.

19. The method of claim 18, wherein said coronavirus is SARS-CoV-2.

20. A method of treating muscle atrophy in a subject with increased circulating levels of IL-1β when compared to a control subject, said method comprising administering an effective amount of a NELL1 polypeptide, or a nucleic acid molecule encoding the same.

21. The method of claim 20, wherein said subject is a pediatric subject.

22. The method of claim 20 or 21, wherein said subject has a cancer.

23. The method of claim 22, wherein said cancer is a stage III or IV cancer.

24. The method of claim 22 or 23, wherein said cancer is a pancreatic or gastric cancer.

25. The method of any one of claims 20-24, wherein said subject has chronic systemic inflammation.

26. The method of claim 25, wherein said chronic systemic inflammation is due to a viral infection.

27. The method of claim 26, wherein said viral infection is by a coronavirus.

28. The method of claim 27, wherein said coronavirus is SARS-CoV-2.

29. The method of any one of claims 20-28, wherein said subject has a genetic predisposition for increased circulating levels of IL-1β when compared to a control subject.

30. The method of any one of claims 20-29, wherein said subject has increased circulating levels of at least one of IL-8, NF-κB, and MMP1 when compared to a control subject.

31. A method of treating muscle atrophy in a subject with a cancer, said method comprising administering an effective amount of a NELL1 polypeptide, or a nucleic acid molecule encoding the same.

32. The method of claim 31, wherein said cancer is a stage III or IV cancer.

33. The method of claim 31 or 32, wherein said cancer is a pancreatic or gastric cancer.

34. The method of any one of claims 31-33, wherein said subject has increased circulating levels of IL-1β when compared to a control subject.

35. The method of any one of claims 31-34, wherein said subject has a genetic predisposition for increased circulating levels of IL-1β when compared to a control subject.

36. The method of any one of claims 31-35, wherein said subject has increased circulating levels of at least one of IL-8, NF-κB, and MMP1 when compared to a control subject.

37. The method of any one of claims 1-36, wherein said NELL1 polypeptide has an amino acid sequence having at least 80% sequence identity to the amino acid sequence set forth as SEQ ID NO: 2.

38. The method of claim 37, wherein said NELL1 polypeptide is the polypeptide of SEQ ID NO: 2.

39. The method of any one of claims 1-36, wherein said NELL1 polypeptide has an amino acid sequence having at least 80% sequence identity to the amino acid sequence set forth as SEQ ID NO: 4.

40. The method of claim 39, wherein said NELL1 polypeptide is the polypeptide of SEQ ID NO: 4.

41. The method of any one of claims 1-36, wherein said NELL1 polypeptide has an amino acid sequence having at least 80% sequence identity to the amino acid sequence set forth as SEQ ID NO: 6.

42. The method of claim 41, wherein said NELL1 polypeptide is the polypeptide of SEQ ID NO: 6.

43. The method of any one of claims 1-36, wherein said NELL1 polypeptide has an amino acid sequence having at least 80% sequence identity to the amino acid sequence set forth as SEQ ID NO: 10.

44. The method of claim 43, wherein said NELL1 polypeptide is the polypeptide of SEQ ID NO: 10.

45. The method of any one of claims 1-36, wherein said NELL1 polypeptide has an amino acid sequence having at least 80% sequence identity to the amino acid sequence set forth as SEQ ID NO: 12.

46. The method of claim 45, wherein said NELL1 polypeptide is the polypeptide of SEQ ID NO: 12.

47. The method of any one of claims 1-36, wherein said NELL1 polypeptide has an amino acid sequence having at least 80% sequence identity to the amino acid sequence set forth as SEQ ID NO: 17.

48. The method of claim 47, wherein said NELL1 polypeptide is the polypeptide of SEQ ID NO: 17.

49. The method of any one of claims 1-36, wherein said NELL1 polypeptide has an amino acid sequence having at least 80% sequence identity to the amino acid sequence set forth as SEQ ID NO: 18.

50. The method of claim 49, wherein said NELL1 polypeptide is the polypeptide of SEQ ID NO: 18.

51. A method of treating muscle atrophy in a subject in need thereof, said method comprising administering an effective amount of a NELL1 polypeptide, or a nucleic acid molecule encoding the same, wherein said NELL1 polypeptide has an amino acid sequence having at least 80% sequence identity to the amino acid sequence set forth as SEQ ID NO: 17 or 18.

52. The method of claim 51, wherein said NELL1 polypeptide has one or more of the properties selected from the group consisting of:
a) enhanced efficacy in tissue regeneration,
b) enhanced prevention of tissue loss,
c) enhanced promotion of wound healing,
d) easier purification,
e) higher yield, and
f) less aggregate formation,
when compared to the NELL1 polypeptide's respective full-length NELL1 protein.

53. The method of claim 51 or 52, wherein said NELL1 polypeptide lacks the carboxy-terminal 179 amino acid residues of the NELL1 polypeptide's respective full-length NELL1 protein.

54. The method of any one of claims 51-53, wherein said subject is a pediatric subject.

55. The method of any one of claims 51-54, wherein said subject has chronic systemic inflammation.

56. The method of claim 55, wherein said chronic systemic inflammation is due to a viral infection.

57. The method of claim 56, wherein said viral infection is by a coronavirus.

58. The method of claim 57, wherein said coronavirus is SARS-CoV-2.

59. The method of any one of claims 51-58, wherein said subject has increased circulating levels of IL-1β when compared to a control subject.

60. The method of any one of claims 51-59, wherein said subject has a genetic predisposition for increased circulating levels of IL-1β when compared to a control subject.

61. The method of any one of claims 51-60, wherein said subject has increased circulating levels of at least one of IL-8, NF-κB, and MMP1 when compared to a control subject.

62. The method of any one of claims 51-61, wherein said subject has a cancer.

63. The method of claim 62, wherein said cancer is a stage III or IV cancer.

64. The method of claim 62 or 63, wherein said cancer is a pancreatic or gastric cancer.

65. A method for treating muscle atrophy or cachexia in a subject having systemic inflammation due to a viral infection.

66. The method of claim 65, wherein said viral infection is by a coronavirus.

67. The method of claim 66, wherein said coronavirus is SARS-CoV-2.

68. The method of any one of claims 1-67, wherein said muscle atrophy is skeletal muscle atrophy and/or cardiac muscle atrophy.

69. The method of any one of claims 1-68, wherein said administering comprises intravenous, subcutaneous, intramuscular, intra-arterial, or intraperitoneal administration.

70. The method of claim 69, wherein said administering comprises local administration to a muscle.

71. The method of any one of claims 1-70, wherein said nucleic acid molecule is comprised within an expression vector and operably linked to a promoter.

72. The method of any one of claims 1-71, wherein said NELL1 polypeptide, or said nucleic acid molecule encoding the same, is incorporated into a drug eluting device, scaffold, matrix, or sutures.

73. The method of any one of claims 1-72, wherein said subject is a mammal.

74. The method of claim 73, wherein said mammal is a human.

75. The method of claim 73, wherein said mammal is a cat, dog or horse.
